(19) United States
(12) Patent Application Publication
(10) Pub. No.: US 2023/0364602 A1
(43) Pub. Date: Nov. 16, 2023

(54) SYSTEMS AND METHODS FOR ALLERGEN DETECTION

(71) Applicant: DOTS Technology Corp., Natick, MA (US)

(72) Inventors: Adi Gilboa-Geffen, Wayland, MA (US); Brian Christopher Burke, Mahwah, NJ (US)

(21) Appl. No.: 18/029,996

(22) PCT Filed: Oct. 13, 2021
(86) PCT No.: PCT/US21/54658

§ 371 Date: Apr. 3, 2023

Related U.S. Application Data
(60) Provisional application No. 63/252,760, filed on Oct. 6, 2021.
(60) Provisional application No. 63/134,223, filed on Jan. 6, 2021.
(60) Provisional application No. 63/091,735, filed on Oct. 14, 2020.
(60) Provisional application No. 63/090,878, filed on Oct. 13, 2020.

(51) Int. Cl. B01L 3/00; G01N 1/28; G01N 21/64

(52) U.S. Cl. CPC B01L 3/502; G01N 1/286; G01N 21/645; B01L 2200/10; B01L 2300/044; B01L 2300/0672; B01L 2300/0681; B01L 2300/087; B01L 2400/0644; G01N 2201/0221

(57) ABSTRACT

The present disclosure is drawn to devices and systems for target detection in samples (e.g., food samples and clinical samples). The allergen detection system includes a sampler, a disposable analytic cartridge and a detection device with an optimized optical system. The allergen detection utilizes nucleic acid molecules as detection agents and detection probes.

### CROSS REFERENCE TO RELATED APPLICATIONS

This application claims the benefit of priority to US Provisional Application Nos.: 63/090,878, filed on Oct. 13, 2020; 63/091,735, filed on Oct. 14, 2020; 63/134,223, filed on Jan. 6, 2021; and 63/252,760, filed on Oct. 6, 2021; the contents of each of which are incorporated herein by reference in their entirety.

### FIELD OF THE DISCLOSURE

The present disclosure is drawn to portable devices and systems, assays for target detection in samples, for example, allergen detection in food samples.

### BACKGROUND OF THE DISCLOSURE

Allergy (e.g., food allergy) is a common medical condition. There are 15 million people with food allergies in the United States, and allergic reactions lead to approximately 200,000 emergency department visits and 200 deaths each year. Strict avoidance of allergens in the diet is the sole treatment for food allergies, but due to the ubiquity of the most common allergens in the food chain, the risk of accidental exposure is high. Food allergy management requires individuals and caregivers to continuously manage exposure to allergens, and food prepared and consumed outside the home can be especially hazardous due to cross-contamination and lack of awareness and knowledge about food allergies among restaurants. Due to these dangers, children and families experience psychological, social, and economic burdens and caregivers of children with food allergies often experience diminished quality of life, anxiety, and frustration over lack of food allergy awareness.

A portable device that enables a person who has food allergy to test their food and determine accurately and immediately the allergen content will be of great benefit to provide for an informed decision on whether to consume or not.

Researchers have tried to develop suitable devices and methods to meet this need, such as those devices and systems disclosed in U.S. Pat. No. 5,824,554 to McKay; US Patent Application Pub. No.: 2008/0182339 and U.S. Pat. No. 8,617,903 to Jung et al.; US Patent Application Pub. No.: 2010/0210033 to Scott et al; U.S. Pat. No. 7,527,765 to Royds; U.S. Pat. No. 9,201,068 to Suni et al.; and U.S. Pat. No. 9,034,168 to Khattak and Sever. There is still a need for improved molecule detection technologies. There is also a need for devices and systems that detect allergens of interest in less time, with high sensitivity and specificity, and with less technical expertise than the devices used today.

The present disclosure provides a portable assembly and a device for fast and accurate detection of an allergen in a sample by using aptamer-based signal polynucleotides (SPNs). The SPNs, as detection agents, specifically bind to the allergen of interest, forming SPN: protein complexes. The complexes are them detected and measured by a detection sensor. The sensor to capture the SPNs may comprise a chip printed with nucleic acid molecules that hybridize to the SPNs (e.g., DNA chip). The detection system may comprise a separate sampler, disposable cartridges/vessels for processing the sample and implementing the detection assay, and a detector unit including an optical system for operating the detection and detecting the reaction signal. The detection agents (e.g., SPNs) and sensors (e.g., DNA chips) may be integrated into the disposable cartridges of the present disclosure. The cartridges, detection agents and the detection sensors may also be used in other detection systems. Other capture agents such as antibodies specific to allergen proteins may also be used in the present detection systems. Such devices may be used by consumers in non-clinical settings, for example in the home, in restaurants, school cafeteria and food processing facilities.

The disclosed platform can empower consumers to easily and quickly assess the presence of allergens in foods before eating to help avoid and alleviate anxiety associated with accidental exposure to allergens as well as related health risks, costs, and emotional burdens.

### SUMMARY OF THE DISCLOSURE

The present disclosure provides systems, devices, disposable cartridges/vessels, optical systems and methods for use in detection of a molecule of interest (e.g., allergen) in various types of samples, particularly food samples. The allergen detection devices and systems are portable and handheld.

An aspect of the present disclosure is an assembly for detecting a molecule of interest of in a sample, for example, an allergen in a food sample. The assembly comprises an analytical cartridge configured to accept the sample for processing to a state permitting the molecule of interest to engage in an interaction with a detection agent. The assembly includes a detector unit configured to accept the analytical cartridge in a configuration which permits a detection mechanism housed by the detector unit to detect the interaction of the molecule of interest with the detection agent. The interaction triggers a visual indication on the detector unit that the molecule of interest is present or absent in the sample. The detector unit may be removably connected to the analytical cartridge.

In some embodiments, the assembly may further comprise a separate sampler configured to collect a sample for detection of the molecule of interest in the sample. In some embodiments, the sampler is a food corer. The corer may be operatively connected to the analytical cartridge to transfer the collected sample to the cartridge.

In some embodiments, the analytical cartridge is disposable, and configured to detect one particular molecule of interest, for example, one allergen. In other embodiments, the analytical cartridge may be configured to detect a plurality of molecules of interest in a sample, for example, a set of allergens.

In some embodiments, the analytical cartridge comprises a homogenizer configured to produce a homogenized sample, thereby releasing the molecule of interest from a matrix of the sample into an extraction buffer that optionally includes the detection agent. The analytical cartridge also comprises a first conduit to transfer the homogenized sample with or without the detection agent through a filter system to provide a filtrate containing the molecule of interest, or the complexes of the molecule of interest and the detection agent, and a second conduit to transfer the filtrate, making the filtrate to be contacted with a detection probe, thereby permitting an interaction of the detection agent with the detection probe. The first and second conduits comprise a plurality of fluidic paths connecting different parts of the conduits from transferring the processed sample, buffers, filtrate, detection agents, waste and other fluids.

In some embodiments, the analytical cartridge may further comprise a rotary valve system providing a mechanism for controlling the transfer of the sample and other fluidic components (e.g., buffers, filtrate, reagents and waste) within the analytical cartridge. The rotary valve system may control the flow rate and the volume of fluid. The rotary valve switching system may be further configured to provide a closed position to prevent fluid movement in the analytical cartridge.

In some embodiments, the homogenizer and the rotary valve system may be powered by motors located in the detector unit when the analytical cartridge is accepted by the detector unit.

In some embodiments, the analytical cartridge comprises a plurality of chambers. The chambers are separate but connected for operation. As a non-limiting example, the analytical cartridge may include a sample processing chamber, a detection chamber, a waste chamber, and optionally a buffer chamber. In some embodiments, the analytical cartridge may further comprise a separate filtrate chamber to hold the filtrate and optionally further concentrate the filtrate prior to the transfer to the detection chamber. In some examples, the detection chamber comprises a detection sensor and an optical window. The detection mechanism of the detector unit analyzes the detection reaction through the optical window to identify the interaction of the molecule of interest with the detection agent in the detection chamber.

In some embodiments, the analytical cartridge comprises a detection sensor for measuring the interaction between the molecule of interest and the detection agent. The detection sensor is included in the detection chamber. In one non-limiting example, the detection sensor is a separate substrate which includes a plurality of fluidic channels and a detector chip area. The substrate is also referred to as a chipannel, wherein the fluidic channels and the detector chip area are connected. In some examples, the chipannel is a plastic substrate.

In some embodiments, the detector chip area within the chipannel comprises at least one reaction panel and at least one control panel. In other embodiments, the detector chip area within the chipannel may comprise one reaction panel and two control panels. In other embodiments, the chipannel may comprise a plurality of reaction panels and a plurality of control panels. Optionally, the detector chip area further comprises one or more fiducial spots that guide image processing by an imaging mechanism (e.g., a camera) of the detector unit. Any suitable fiducial object may be spotted as a fiducial marker for reference.

In some embodiments, the detector chip area within the chipannel comprises a detection probe molecule immobilized on the reaction panel. The detection probe is configured to engage in a probe interaction with the detection agent. An interaction of the molecule of interest with the detection agent prevents the detection agent from engaging in the probe interaction with the detection probe. The detector chip area within the chipannel may further include an optically detectable control probe molecule immobilized on the control panel(s), for normalization of signal output measured by the detection mechanism.

In an embodiment, the chipannel is a plastic chip wherein the reaction panel is printed with a nucleic acid-based detection probe that comprises a nucleic acid sequence complementary to nucleic acid sequence of the detection agent and wherein the control panel is printed with nucleic acid based control probe molecule that does not bind to the molecule of interest or the detection agent.

The analytical cartridge may further include a chamber storing wash buffer for washing the detection chamber and a waste chamber for accepting outflow contents of the detection chamber after washing. In some embodiments, the series of bridging fluid conduits may comprise: (a) a fluid connection between the wash buffer chamber and the detection chamber; and (b) a fluid connection between the detection chamber and the waste chamber.

In some embodiments, the filter in the analytical cartridge is a filter assembly comprising a bulk filter and a membrane filter. The bulk filter may comprise a gross filter and a depth filter. In some embodiments, the filter assembly may further comprise a filter cap that can lock the rotary valve.

In some embodiments, the molecule of interest in the homogenized sample may be brought in contact with the detection agent prior to the molecule of interest and detection agent in contact with the detector probe. The contact of the molecule of interest and detection agent may occur in the extraction buffer during homogenization, or in the filter during the filtration, or in the filtrate chamber. In some embodiments, a MgCl2 deposit, such as MgCl2 containing lyophilized bead, is prestored in the filter or in the filtrate chamber.

In some embodiments, the analytical cartridge may comprise a data chip unit configured for providing the cartridge information.

In an embodiment an analytic cartridge for detecting a molecule of interest in a sample comprising a first compartment with a homogenizer for receiving a sample and processing the sample. The homogenizer configured to produce a homogenized sample, thereby releasing the molecule of interest from a matrix of the sample into an extraction buffer in the presence of the detection agent and permitting the molecule of the interest in the sample to engage in the interaction with the detection agent. The cartridge includes a lid covering the cartridge, and the lid comprises at least one aperture opening into the first compartment, a cap rotatably connected to the lid, wherein the cap is capable of rotating from a first position to a second position, a seal on the at least one aperture creating a pocket between the seal and the cap, a homogenization accelerator positioned in the pocket when the cap is in a first position, and wherein when the cap is rotated to the second position the homogenization accelerator is released into the first compartment. The cartridge includes a conduit to transfer the homogenized sample and detection agent through a filter system to provide a filtrate containing the molecule of interest and the detection agent. The cartridge includes a second compartment for contacting the filtrate containing the molecule of interest and the detection agent with detection probes; the second compartment comprising a transparent substrate that comprises fluidic channels and a detection chip area with a detection probe immobilized thereon, the detection probe configured to engage in a probe interaction with the detection agent, wherein the interaction of the molecule of interest with the detection agent prevents the detection agent from engaging in the probe interaction with the detection probe.

The cartridge also includes a rotary valve system configured to regulate the transfer of the homogenized sample and detection agent through the filter system, of the filtrate to the second compartment, and of wash buffer to the second compartment and outflow contents from the second compartment to a waste chamber, a compartment for holding wash buffer for washing the detection area, and a waste chamber for accepting outflow contents of the detection chamber. The at least one aperture further includes a second aperture opening into the first compartment.

The cap further includes a port which, when the cap is in the first position, co-localizes with the second aperture; the second aperture containing a breakable seal facing the first compartment. When the cap is in the second position, the second aperture is covered by the cap and scaled by a movable cover. The cartridge may be used in combination with a detector device comprising an external housing configured for providing support for the components of the detection device. The components integrated for operating a detection test comprising an assembly lid capable of measuring the weight, mass, or volume of a sample, a motor for driving and controlling the sample homogenization, a motor for controlling a valve system, a pump for driving and controlling fluidic flow, an optical system for detecting fluorescence signals, means for converting and digitizing the fluorescence signals, a display window for receiving the detected signals and indicating the presence and/or absence of the allergen in the test sample, and a power supply.

An aspect of the disclosure includes a test cup assembly for processing a sample to a state permitting detection of a molecule of interest in the sample comprising a top cover for sealing the test cup and providing an identification label, the top cover further comprising: a movable cap, the movable cap having a lancing element, and a homogenization accelerator, which is secured in a pocket bounded by the cap and a sealed aperture on the top cover. The test cup includes a body part for receiving and processing the sample to a state permitting the molecule of interest in the sample to engage in an interaction with a detection agent. The body part comprises a first compartment with a homogenizer for homogenizing the sample to extract the molecule of interest using an extraction buffer, thereby releasing the molecule of interest from a matrix of the sample into the extraction buffer and engaging in the interaction with a detection agent present in the extraction buffer. The test cup includes a conduit for transferring the homogenized sample containing the molecule of the interest and detection agent through a filter system to provide a filtrate containing the molecule of interest and the detection agent, a chamber for holding wash buffer, a waste chamber for receiving and storing the outcome contents after washing the molecule of interest and the detection agent, and a rotary valve system for controlling the fluid movement inside the test cup assembly. The test cup includes a transparent substrate comprising a plurality of fluidic channels and a detection area with a detection probe immobilized thereon, the detection probe configured to engage in a probe interaction with the detection agent. The interaction of the molecule of interest with the detection agent prevents the detection agent from engaging in the probe interaction with the detection probe. The test cup also includes a bottom cover for sealing the test cup and providing an interface to connect the test cup to a detector unit for operating the detection. The bottom cover comprising a transparent window that is aligned with the detection area of the transparent substrate upon assembly of the test cup.

The disposable analytical cartridge comprises a lid with a movable cap, the movable cap having a lancing element, and a homogenization accelerator, which is secured in a pocket bounded by the cap and a seal on the lid. The cartridge includes a sample processing chamber with a homogenizer configured to homogenize the sample with an extraction buffer in the presence of the detection agent, thereby permitting the allergen of the interest in the sample to engage in the interaction with the detection agent. The cartridge includes a filter system configured to provide a filtrate containing the allergen of interest and the detection agent. The cartridge includes a separate transparent substrate comprising a plurality of fluidic channels and a detection area with a detection probe molecule immobilized thereon; the detection probe configured to engage in a probe interaction with the detection agent, wherein the interaction of the molecule of interest with the detection agent prevents the detection agent from engaging in the probe interaction with the detection probe. The cartridge includes a detection chamber with an optical window, a chamber holding wash buffer for washing the substrate and the detection chamber, a waste chamber for accepting and storing outflow contents of the detection chamber after wash. The cartridge includes a rotary valve system and conduits configured to transfer the homogenized sample and detection agent through the filter system, to transfer the filtrate to the detection chamber, and to transfer the wash buffer to the detection chamber and outflow contents from the detection chamber to the waste chamber, and an air flow system configured to regulate air pressure and flow rate in the cartridge.

The movable cap is rotatably secured to the lid, the lid comprising an aperture opening into the homogenization chamber, the pocket being adjacent with the aperture. Movement of the movable cap from a first position to a second position causes the lancing element to lance the seal allowing the homogenization accelerator to enter the homogenization chamber.

The detection device includes a frame attachable to the housing a base attached to the frame and a cover connected to the frame; wherein the cover includes a measurement device adjacent thereto and above the base. The measurement device is capable of detecting and measuring the weight, mass, or volume of the sample when the sample is placed on the cover. The measurement device is a strain gauge.

An aspect of the embodiment includes a method for detecting the presence or absence of a molecule of interest in a sample comprising collecting a sample, measuring the weight of the sample, homogenizing the sample with an accelerator, and processing the sample in an extraction buffer in the presence of a detection agent, thereby permitting the interaction of the molecule of interest with the detection agent. The method further includes filtrating the processed sample containing the molecule of interest and the detection agent, contacting the filtrate with a substrate with a detection probe immobilized thereon; the detection probe configured to engage in a probe interaction with the detection agent, wherein the interaction of the molecule of interest with the detection agent prevents the detection agent from engaging in the probe interaction with the detection probe. The method includes washing off the unbound compounds from the substrate with a wash buffer, measuring fluorescence signals from the substrate, and detecting the presence or absence of the molecule of interest in the sample.

In some embodiments, the assembly of the present disclosure comprises a detector unit that is operatively connected to an analytical cartridge. In some embodiments, the detector unit of the assembly comprises a detection mechanism to measure detection signals. i.e., the interaction between the detection agent and detector probe. As a non-limiting example, the detection mechanism is an imaging system, such as a camera for fluorescence imaging.

In some embodiments, the detector unit of the assembly comprises an external housing that provides support for the components integrated for operating a detection reaction and measuring detection signals, of the detector unit and for accepting the analytical cartridge. In accordance with the present disclosure, the components for operating a detection reaction and measuring detection signals include motors for driving and controlling the homogenization, and controlling the rotary valve; pump driving and controlling the fluidic flow of the processed sample, the filtrate, buffers and waste in the compartments of the analytical cartridge; an optical system for detecting and visualizing a detection result; and a display window.

In some embodiments, the optical system may comprise excitation optics and emission optics and an optical reader. The optical system is modified for detecting signals from the detector chip area of the chipannel within the cartridge.

In other embodiments, the optical system may comprise a camera sensor (e.g., a CCD camera and a sCMOS camera) to generate images of a detection reaction of the detector chip area of the chipannel. The images are then processed to indicate the detection results.

In some embodiments, the detection assembly may comprise a user interface that may be accessed and controlled by a software application. The software may be run by a software application on a personal device such as a smartphone, a tablet computer, a personal computer, a laptop computer, a smartwatch, and/or other devices. In some embodiments, the personal device runs on iOS or Android software. In some cases, the software may be run by an internet browser. In some embodiments, the software may be connected to a remote and localized server referred to as the cloud. The personal device and software may record test results and allow for community interaction. The interaction may include a physician being able to view the data and usage of the device by a patient. The interaction may also include a parent or family member being able to view the date and usage by a child or other family member.

An aspect of the disclosure includes an assembly for detecting a molecule of interest in a sample comprising a sample processing cartridge having a homogenization chamber configured to accept the sample for processing to a state permitting the molecule of interest to engage in an interaction with a detection agent. The cartridge comprises a lid, a movable cap having a lancing element, and a homogenization accelerator, which is secured in a pocket bounded by the cap and a seal on the lid. The assembly also includes a detector unit configured to accept the sample processing cartridge in a configuration which permits a detection mechanism housed by the detector unit to detect the interaction of the molecule of interest with the detection agent, wherein the interaction triggers a visual indication on the detector unit that the molecule of interest is detected. The visual indication is by processing images capturing the interaction of the molecule of interest with the detection agent. The movable cap is secured to the lid and further comprises at least one aperture opening into the homogenization chamber. The pocket of the assembly is co-located with the at least one aperture. Movement of the movable cap causes the lancing element to lance the seal allowing the homogenization accelerator to enter the homogenization chamber. The at least one aperture further includes a second aperture opening into the homogenization chamber. The cap further includes a port which, in a first position of the cap, co-localizes with the second aperture; the second aperture containing a breakable seal facing the homogenization chamber. In a second position of the cap the second aperture is covered by the cap and sealed by a movable cover.

The assembly further comprises an assembly lid capable of measuring the weight, mass, or volume of a sample. The assembly lid further comprises a frame, a base attached to the frame, and a cover connected to the frame. The cover includes a measurement device adjacent thereto and above the base, whereby the measurement device is capable of detecting and measuring the weight, mass, or volume of the sample when the sample is placed on the cover. The measurement device is a strain gauge or a load gauge.

In a non-limiting embodiment of the present disclosure, a detection assembly comprises an analytical cartridge that is configured to be a disposable test cup or cup-like container, a detector unit comprising a docket for accepting the test cup, and an optional sampler. The disposable test cup or cup-like container may be constructed as an analytical module in which a sample is processed and a molecule of interest in the test sample (e.g., an allergen) is detected through the interaction with a detection agent.

In some embodiments, the disposable test cup or cup-like container comprises a top cover configured to accept the sample and to seal the cup or cup-like container wherein the top cover includes a port for accepting the sample and at least one breather filter that allows air in; a body part configured to process the sample to a state permitting the molecule of interest to engage in an interaction with the detection agent; and a bottom cover configured to connect to the cup body part thereby forming a detection chamber with an optical window at the bottom of the test cup, and to provide the connecting surface to a detector unit. The exterior of the bottom cover comprises a plurality of ports for connecting a plurality of motors located in the detector unit to operate the homogenizer, the rotary valve system and the flow of the fluids. The optical window of the detection chamber is connected to the detection mechanism in the detector unit. In some embodiments, the test cup or cup-like container further comprises a detection sensor such as a transparent substrate with detection probes immobilized thereon. The transparent substrate is a chipannel comprising a detection chip area with nucleic acid-based probes immobilized thereon and fluidic paths.

In one non-limiting embodiment of the present disclosure, the disposable test cup comprises (a) a first compartment with a homogenizer for receiving a sample and processing the sample; the homogenizer configured to produce a homogenized sample, thereby releasing the molecule of interest from a matrix of the sample into an extraction buffer in the presence of the detection agent and permitting the molecule of the interest in the sample to engage in the interaction with the detection agent; (b) a second compartment for contacting the filtrate containing the molecule of interest and the detection agent with detection probes; the second compartment comprising a chipannel that comprises a plurality of fluidic channels and a detection chip area with the detection probes immobilized thereon; (c) a conduit to transfer the homogenized sample and detection agent through a filter system to provide a filtrate containing the molecule of interest and the detection agent; (d) a rotary valve system configured to regulate the transfer of the homogenized sample and detection agent through the filter system, of the filtrate to the second compartment, and of wash buffer to the second compartment and outflow contents from the second compartment to a waste chamber; (e) a compartment for holding wash buffer for washing the detection area; and (f) a waste chamber for accepting outflow contents of the detection chamber. In some examples, the detection probe is configured to engage in a probe interaction with the detection agent, wherein the interaction of the molecule of interest with the detection agent prevents the detection agent from engaging in the probe interaction with the detection probe. The fluidic paths within the chipannel transfer the filtrate, making the filtrate to be contacted with the detection probe immobilized on the chip area, and transfer the outflow contents to the waste chamber.

In some embodiments, the cup top cover further comprises a layer for providing an identification label.

In some embodiments, the parts of the disposable test cup are molded together forming an analytic module.

Another aspect of the present disclosure relates to a method for detecting the presence and/or absence of a molecule of interest in a sample comprising the steps of (a) collecting a sample suspected of containing the molecule of interest, (b) homogenizing the sample in an extraction buffer in the presence of a detection agent, thereby releasing the molecule of interest from the sample to engage in an interaction with the detection agent comprising a fluorescent moiety, (c) filtrating the homogenized sample containing the molecule of interest and the detection agent; (d) contacting the filtrate containing the molecule of interest and the detection agent with a detection probe molecule that engages in a probe interaction with the detection agent, wherein the interaction of the molecule of interest with the detection agent prevents the detection agent from engaging in the probe interaction with the detection probe; (e) washing off the contact in step (d) with wash buffer; (f) measuring signal outputs from the probe interaction of the detection probe molecule and the detection agent; and (g) processing the detected signals and visualizing the interaction between the detection probe and the detection agent.

The molecule of interest may include, but is not limited to, a protein and a variant or fragment thereof, a nucleic acid molecule (e.g., a DNA or RNA molecule) or a variant thereof, a lipid, a sugar and a small molecule. In some embodiments, the molecule of interest may be a protein, or variant and fragment thereof. In one example, the molecule of interest is an allergen such as a food allergen. The detection agent may be an antibody or variant thereof, a nucleic acid molecule or variant thereof, or a small molecule. In some embodiments, the detection agent is a nucleic acid molecule comprising a nucleic acid sequence that binds to the molecule of interest. In one example, the nucleic acid-based detection agent is a signaling polynucleotide (SPN) derived from an aptamer comprising a core nucleic acid sequence that binds to the molecule of interest. The SPN may further comprising a detectable moiety such as a fluorescent moiety. Accordingly, the detection probe may comprise a complementary nucleic acid sequence that hybrids to the free sequence of the SPN.

### BRIEF DESCRIPTION OF THE DRAWINGS

FIG. 1 is a perspective view of an embodiment of a detection system in accordance with the present disclosure comprising a detection device 100 having an external housing 101 and a port or receptacle 102 configured for holding the disposable cartridge 300, a separate food corer 200 as an example of the sampler, a disposable test cup 300 as an example of the analytical cartridge. Optionally, a lid 103, execution/action button 104 that allows a user to execute an allergen detection testing and a USB port 105 may be included.

FIG. 2A is an exploded perspective view of one embodiment of the food corer 200 as an example of the sampler.

FIG. 2B is a perspective view of the sampler assembly 200.

FIG. 3A is a perspective view of an embodiment of a disposable test cup 300, comprising a cup top 310, a cup body 320 and a cup bottom 330.

FIG. 3B is a cross-sectional view of the test cup 300, illustrating features inside the cup 300.

FIG. 3C is an exploded view of the disposable test cup 300.

FIG. 3D is a top (left panel) perspective view and a bottom (right panel) perspective view of the top cover 312.

FIG. 3E is an exploded view of the cup top lid 311.

FIG. 3F is a top perspective view (left panel) and a bottom perspective view (right panel) of the cup body 320.

FIG. 3G is a bottom perspective view of the bottom of the upper housing 320a (upper panel) shown in FIG. 3C and a top perspective view of the inside of the outer housing 320b (lower panel) shown in FIG. 3C.

FIG. 3H is a bottom perspective view (left panel) and a top perspective view (right panel) of the cup bottom cover 337.

FIG. 3I is a bottom perspective view of the cup bottom surface after assembling the bottom 330 and the cup body 320.

FIG. 4A is an exploded view of one embodiment of the filter assembly 325.

FIG. 4B is a cross-sectional perspective view of one embodiment of the filtrate chamber 322 comprising a filter bed chamber 431 for placement of the filter assembly 325, a collection gutter 432 and a filtrate collection chamber 433.

FIG. 5A is a perspective view of an alternative embodiment of the cup 300.

FIG. 5B is an exploded view of the disposable test cup 300 of FIG. 5A (the filter 325 not shown).

FIG. 5C is a cross sectional perspective view of the cup 300 of FIG. 5A.

FIG. 6A is an exploded view of an alternative embodiment of the cup 300.

FIG. 6B is a top perspective view (right panel) and a bottom perspective view (left panel) of the cup body 320 of FIG. 6A.

FIG. 6C is a bottom perspective view of the cup bottom 337 and the bottom of the cup body 320 of FIG. 6A.

FIG. 6D is an alternative embodiment of the filter assembly 325.

FIG. 6E is a cross-sectional view of the filter cap 621 when is assembled with the rotary valve 350.

FIG. 6F is a perspective view of the rotary valve 350 (upper panel) and a bottom perspective view of the bottom of the rotary valve 350 (lower panel).

FIG. 6G is a bottom perspective view (upper panel) and a top perspective view (lower panel) of the cup bottom cover 337 shown in FIG. 6A.

FIG. 7A is an exploded view of an alternative embodiment of the cup 300; the cup 300 comprises a chipannel 710.

FIG. 7B is a perspective view of the chipannel 710 shown in FIG. 7A.

FIG. 7C is a bottom perspective view of the chipannel 710.

FIG. 7D is a bottom perspective view of an alternative embodiment of the chipannel 710.

FIG. 7E is exploded view of an alternative embodiment of the cup 300.

FIG. 7F is an alternative embodiment of the cup body in which the filter gasket 623 is overmolded to the cup body.

FIG. 7G is an alternative embodiment of the rotary valve 350 shown in FIG. 7E.

FIG. 7H is another alternative embodiment of the rotary valve 350′.

FIG. 7I is a cross-sectional view of the cup body 320 shown in FIG. 7E, showing the overmolded seal 713 to combine several parts into a single part.

FIG. 7J is an alternative embodiment of the cup bottom cover 337 with compression coil springs 721.

FIG. 7K is perspective views of the cup bottom cover 337 shown in FIG. 7J, demonstrating the compression coil springs 721 at the bottom.

FIG. 7L is a perspective view of the sacrificial weld bead material 722 in the bottom of the cup body 320 shown in FIG. 7E.

FIG. 8A is a top perspective view of the cup body 320 showing features relating to homogenization, filtration (F), wash (W1 and W2) and waste.

FIG. 8B is a scheme showing the positions of the rotary valve 350 during the sample preparation and sample washes.

FIG. 8C is a diagram displaying the fluid flow inside the cup 300.

FIG. 9A is a perspective view of the device 100

FIG. 9B is a top perspective view of the device 100 in the absence of the lid 103.

FIG. 10A is a longitudinal cross-sectional view of the device 100.

FIG. 10B is a lateral cross-sectional view of the device 100.

FIG. 11A is a valve motor 1020 and associated components for controlling the operation of the rotary valve 350.

FIG. 11B is a top perspective view of the output coupling 1020 associated with the motor.

FIG. 12A is a top perspective view of one embodiment of the optical system 1030.

FIG. 12B is a side view of the optical system 1030 of FIG. 12A.

FIG. 13A is an illustration of a chip sensor 333 displaying the test area and control areas.

FIG. 13B is a top view of the optical system 1030 and chip 333 showing reflections providing fluorescence measurements of the chip 333.

FIG. 13C is a perspective view of another embodiment of the chip senor 333 or the sensing area 333′ of the chipannel 710 displaying one reaction panel 1312, one control panel 1313 and two fiducial panels 1311.

FIG. 13D shows an exemplary pattern of the probes in the reaction panel and control panel of the detection area 333′ of the chipannel 710.

FIG. 14A shows the optical assembly 1030 in a straight mode.

FIG. 14B shows the optical assembly 1030 in a folded mode.

FIG. 14C is a cross-sectional perspective view of one end of the device 100 (right side of FIG. 10B) showing emission optics 1420 including lenses 1421, 1423 and filters 1422a and 1422b placed in the stepped bore 1480 in the device 100.

FIG. 15A is a perspective view of another embodiment of the optical system 1030 comprising an excitation optics 1510, an emission optics 1520 and a camera-based detector 1530.

FIG. 15B is a cross sectional view of the optical components of FIG. 15A as the optical system is configured inside the detection device 100.

FIG. 16A is a histogram demonstrating the SPN intensity in a MgCl₂ lyophilized formulation as compared to the buffer without MgCl₂ and the MgCl₂ solution.

FIG. 16B shows the percentage of magnesium recovered from MgCl₂ formulations deposited on the cotton filter supported on 1 μm mesh.

FIG. 17A shows an expanded view of an embodiment with a rotatable cap 1700 with bead 1703.

FIG. 17B shows a cross-section of the assembled embodiment in FIG. 17A.

FIG. 17C is a side plan view of the cross-section in FIG. 17B.

FIG. 18A is an expanded view of an embodiment with an integrated 1800 lid with a scale.

FIG. 18B is a side plan view of the device in FIG. 18A.

FIGS. 19A-C show the determination of dissociation constants for peanut aptamers for Ara Ha protein (19A), peanut butter (19B) and peanut flour (19C).

FIG. 20 depicts a fluorescently labeled aptamer and its interaction with peanut protein and the probes printed on the detector chip.

FIGS. 21A-D depict the specificity of a peanut specific aptamer P1-16.

FIG. 22A depicts the interaction of an allergen specific aptamer with detection probes and control probes.

FIG. 22B shows a representative image of the detection chip.

FIGS. 23A-B shows assay validation in different food components and additives.

FIGS. 24A-C shows use of gluten specific aptamer for binding of gluten.

FIGS. 25A-C shows aptamers binding to complementary anchors (i.e., detection probes).

FIG. 26A is a view of an embodiment of the detection unit (i.e., instrument) and the analytical cartridge (i.e., test pod) for assay. FIG. 26B is a view of the test pod showing the reaction changer and homogenizer (i.e., blender) in the sample chamber.

FIG. 27 is a depiction of false negatives.

FIG. 28 is a graph of the sensitivity of the peanut specific aptamer P1-16 in accelerated aging.

FIG. 29 is a graph of the stability of the peanut specific aptamer P1-16 at high temperatures.

FIGS. 30 and 31 are graphs showing the accuracy in 45 and 70-food tests.

FIG. 32 is a flow chart for operation of the system and algorithm.

FIGS. 33A-C is a testing of AraH1 protein in 1 ml urine sample (p<0.05).

FIGS. 34A-C is a testing of AraH1 protein in 1 ml serum sample (p<0.05).

### DETAILED DESCRIPTION OF THE DISCLOSURE

The foregoing has outlined rather broadly the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure. Unless defined otherwise, all technical and scientific terms used herein have the same meaning as commonly understood by one of ordinary skill in the art to which this disclosure belongs. In the case of conflict, the present description will control.

The use of analytical devices to ensure food safety has not yet advanced to the point of fulfilling its promise. In particular, portable devices based on simple, yet accurate, sensitive and rapid detection schemes have not yet been developed for detection of the wide variety of known allergens. One of the more recent reviews of aptamer-based analysis in context of food safety control indicated that while a great variety of commercial analytical tools have been developed for allergen detection, most of them rely on immunoassays. It was further indicated that the selection of aptamers for this group of ingredients is emerging (Amaya-González et al., Sensors 2013, 13, 16292-16311, the contents of which are incorporated herein by reference in their entirety).

The present disclosure provides detection assemblies and systems that can specifically detect low concentrations of allergens in a variety of food samples. The detection system and/or device of the present disclosure is a miniaturized, portable, and hand-held product, which is intended to have a compact size which enhances its portability and discreet operation. A user can carry the detection system and device of the present disclosure and implement a rapid and real-time test of the presence and/or absence of one or more allergens in a food sample, prior to consuming the food. The detection system and device, in accordance with the present disclosure, can be used by a user at any location, such as at home or in a restaurant. The detection system and/or device displays the test result as a standard readout and the detection can be implemented by any user following the simple instructions on how to operate the detection system and device. A specific utility of this detection system is the ease and rapidity of the system. The detection systems and assemblies of the present disclosure may also be used to detect any molecule of interest (i.e., any target) in a sample in general; the molecule of interest may be a protein or a variant thereof, a nucleic acid molecule (e.g., a DNA or RNA molecule) or a variant thereof, a lipid, a sugar, a small molecule, or a cell.

In some embodiments, the detection system is constructed for simple, fast, and sensitive one-step execution from the introduction of the sample to the system. The system may complete a detection test in less than 10 minutes, less than 9 minutes, less than 8 minutes, less than 7 minutes, less than 6 minutes, less than 5 minutes, or less than 4 minutes, or less than 3 minutes, or less than 2 minutes, or less than 1 minute. In some examples, the detection may be completed in approximately 60 seconds, 55 seconds, 50 seconds, 45 seconds, 40 seconds, 35 seconds, 30 seconds, 25 seconds, 20 seconds, or 15 seconds.

In accordance with the present disclosure, the detection system may involve a mechatronic construction process integrating electrical engineering, mechanical engineering and computing engineering to implement and control the process of a target detection test, including but not limiting to, rechargeable or replaceable batteries, motor drivers for processing the test sample, pumps for controlling the flow of the processed sample solutions and buffers within the cartridge, printed circuit boards, and connectors that couple and integrate different components for a fast allergen testing. The detection device of the present disclosure also includes an optical system which is configured for detection of the presence and concentration of a molecule of interest (e.g., an allergen) in a test sample and conversion of detection signals into readable signals; and a housing which provides support for other parts of the detection device and integrates different parts together as a functional product.

In some embodiments, the detection system is constructed such that disposable analytical cartridges (e.g., a disposable test cup or cup-like container), unique to one or more specific molecules of interest (e.g., allergens), are constructed for receiving and processing a test sample and implementing the detection test, in which all the solutions are packed. Therefore, all the solutions may be confined in the disposable analytical cartridges. As a non-limiting example, a disposable peanut test cup may be used to detect peanut in any food sample by a user and discarded after the test. This prevents cross-contamination when different allergen tests are performed using the same device. In some embodiments, a separate sampler for collecting a test sample is provided.

In accordance with the present disclosure, the disposable analytical cartridge comprises detection agents that specifically bind to and recognize an allergen or a molecule of interest. The detection agents may be, but are not limited to, antibodies or variants thereof, nucleic acid molecules or variants thereof, and small molecules. In some embodiments, the detection agents may be nucleic acid molecules comprising nucleic acid sequences that specifically bind to a molecule of interest. The nucleic acid-based detection agents may be aptamers and signaling polynucleotides (SPNs) derived from aptamers that can recognize the target molecule such as an allergen. The aptamers specific to an allergen of interest may be identified using any known aptamer discovery and development methods. The allergen specific aptamers may be further modified and optimized to generate the SPNs. In some embodiments, the aptamers and/or SPNs capture the target molecules in the sample to form SPN:target complexes. Another detection probe comprising short nucleic acid sequences that are complementary to the SPN sequence may be used to anchor the SPN to a solid substrate for signal detection. In other embodiments, the detection agents and detection probes may be attached to a solid substrate such as the surface of a magnetic particle, silica, agarose particles, polystyrene beads, a glass surface, a plastic chip, a microwell, a chip (e.g., a microchip), or the like. It is within the scope of the present disclosure that such detection agents and detection probes can also be integrated into any suitable detection systems and instruments for similar purposes.

### Detection Assemblies and Systems

In accordance with the present disclosure, a detection system or assembly for implementing a detection test of a molecule of interest (e.g., an allergen) in a sample comprises at least one disposable analytical cartridge for processing the sample to a state permitting the molecule of interest to engage in an interaction with a detection agent, and a detector unit for detecting and visualizing the result of the detection (i.e., the interaction between the molecule of interest and the detection agent). Optionally, the detection system may further comprise at least one sampler for collecting a test sample. The sampler can be any tool that can be used to collect a portion of a test sample, e.g., a spoon. In some aspects, a particularly designed sampler may be included to the present detection system as discussed hereinbelow. The exemplary embodiments described below illustrate such detection systems and assemblies for detecting an allergen in a sample.

In general, the analytical cartridge is configured to accept the sample for processing to a state permitting the molecule of interest to engage in an interaction with a detection agent. The detector unit is configured to accept the analytical cartridge in a configuration which permits a detection mechanism housed by the detector unit to detect the interaction of the molecule of interest with the detection agent. The interaction triggers a visual indication on the detector unit that the molecule of interest is present or absent in the sample. The detector unit may be removably connected to the analytical cartridge.

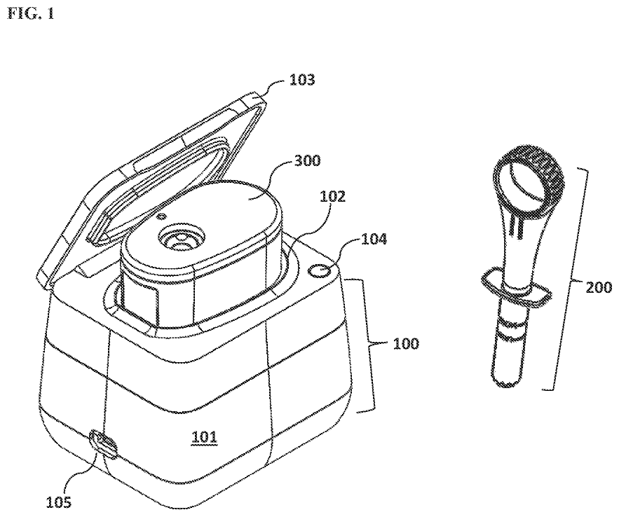

As shown in FIG. 1, an embodiment of the detection system or assembly of the present disclosure comprises a detection device 100 configured for processing a test sample, implementing an allergen detection test, and detecting the result of the detection test, a separate food corer 200 as an example of the sampler, and a disposable test cup 300 as an example of the analytical cartridge. The detection device 100 includes an external housing unit 101 that provides support to the components of the detection device 100. A port or receptacle 102 of the detection device 100 is constructed for docking the disposable test cup 300 and a lid 103 is included to open and close the instrument. The external housing unit 101 also provides surface space for buttons that a user can operate the device. An execution/action button 104 that allows a user to execute an allergen detection testing and a USB port 105 may be included. Optionally a power plug (not shown) may also be included. During the process of implementing an allergen detection test, the food corer 200 with a sample contained therein is inserted into the disposable test cup 300 and the disposable test cup 300 is inserted into the port 102 of the detection device 100 for detection.

### Sampler

Collecting an appropriately sized sample is an important step for implementing allergen detection testing. In some embodiments of the present disclosure, a separate sampler for picking up and collecting test samples (e.g., food samples) is provided. In one aspect, a coring-packer-plunger concept for picking up and collecting a food sample is disclosed herein. Such mechanism may measure and collect one or several sized portions of the test sample and provide pre-processing steps such as cutting, grinding, abrading and/or blending, for facilitating the homogenization and extraction or release of allergen proteins from the test sample. The sampler may be operatively connected to the analytical cartridge and the detection device for transferring a test sample to the cartridge for sample processing. According to the present disclosure, a separate food corer 200 is constructed for obtaining different types of food samples and collecting an appropriately sized portion of a test sample. In one example, the sample is a liquid sample. In another example, the sample is a solid sample.

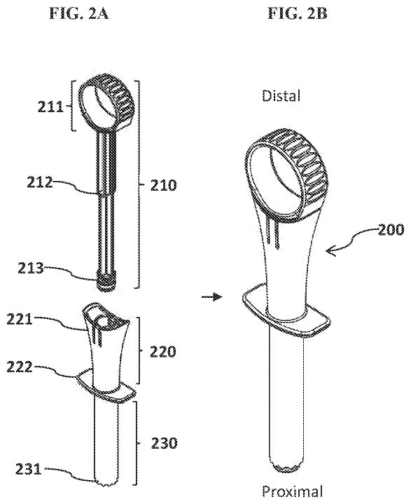

As shown in FIG. 2A, an embodiment of the food corer 200 may comprise three parts: a plunger 210 at the distal end, a handle 220 configured for coupling a corer 230 at the proximal end. The plunger 210 has a distal portion provided with a corer top grip 211 (FIG. 2A) at the distal end, which facilitates maneuverability of the plunger 210 up and down, a plunger stop 212 in the middle of the plunger body, and a seal 213 at the proximal end of the plunger body. The handle 220 may comprise a snap fit 221 at the distal end and a projecting flat collar at the proximal end connecting to the corer 230. In one embodiment, the projecting flat collar comprises a flange 222 as shown in FIG. 2A. The corer 230 may comprise a proximal portion provided with a cutting edge 231 at the very proximal end (FIG. 2A). The corer 230 is configured for cutting and holding the collected sample to be expelled into the disposable test cup 300.

In some embodiments, the distal end of the plunger 210 may comprise a push plate. The plate may be a flat plate, in any shape. In one preferred embodiment, the push plate may be in a rounded square shape with a flared surface. Additionally, the rounded square shape provides an anti-roll feature when the sampler 200 is on a flat surface. This feature also can keep the collected sample inside the corer 230 (i.e., the sample area) from contacting an outside surface (e.g., a table when the sampler is lying on the table).

In some embodiments, the projecting flat collar may be configured as a small circular ring, a rib, or the like. This projection may prevent fingers from sliding down into the sample area and provide tactile orientation as well. As a non-limiting example, the projecting flat collar is a small circular ring.

In one embodiment, the plunger 210 may be inserted inside the corer 230, where the proximal end of the plunger 210 may protrude from the corer 230 for directly contacting a test sample, and together with the cutting edge 231 of the corer 230, picking up a sized portion of the test sample (FIG. 2B). In accordance with the present disclosure, the plunger 210 is used to expel sampled food from the corer 230 into the disposable test cup 300, and to pull certain foods into the corer 230 as well, such as liquids and creamy foods. The feature of the plunger stop 212, through an interaction with the snap fit 221, may prevent the plunger 210 from being pulled back too far or out of the corer body 230 during sampling. The seal 213 at the very proximal end of the plunger 210 may maintain an air-tight seal in order to withdraw liquids into the corer 230 by means of pulling the plunger 210 back. In some embodiments, the plunger 210 may be provided with other types of seals including a molded feature, or a mechanical seal. The handle 220 is constructed for a user to hold the coring component of the sampler 200. For example, the skirt 222 gives the user means for operating the food sampler 200, pushing down the corer 230 and driving the corer 230 into the food sample to be collected.

In some embodiments, the plunger 210 may comprise markings to provide additional guidance to the user, indicating the position of the plunger inside the corer and its position relevant to the minimal and maximal sampling lines. In some embodiments, the lines indicating the minimal and maximal amounts of the sample to be collected are added to the exterior of the corer 230. A user can correct the size of the sampling compartment by adjusting the minimal and maximal lines.

In some embodiments, the cutting edge 231 may be configured for pre-processing the collected sample, allowing the sampled food to be cored in a pushing, twisting and/or cutting manner. The cutting edge 231 may cut a portion from the test sample. As some non-limiting examples, the cutting edge 231 may be in a flat edge, a sharp edge, a serrated edge with various numbers of teeth, a sharp serrated edge and a thin wall edge. In other aspects, the inside diameter of the corer 230 varies, ranging from about 5.5 mm to 7.5 mm. Preferably the inside diameter of the corer 230 may be from about 6.0 mm to about 6.5 mm. The inside diameter of the corer 230 may be 6.0 mm, 6.1 mm, 6.2 mm, 6.3 mm, 6.4 mm, 6.5 mm, 6.6 mm, 6.7 mm, 6.8 mm, 6.9 mm, or 7.0 mm. The size of the corer 230 is optimized for a user to collect a right amount of the test sample (e.g., 1.0 g to 0.5 g).

The parts of the food corer 200 may be constructed as any shape for easy handling such as triangular, square, octagonal, circular, oval, and the like.

In some embodiments, the plunger 210 and the other parts of the sampler may be in different colors. As a non-limiting example, the plunger may be in green color and the corer may be transparent. The increased contrast provides a clear view of the position of the plunger with respect to the sampler. In other embodiments, the food corer 200 may be further provided with a means for weighing a test sample being picked up, such as a spring, a scale or the equivalent thereof. As a non-limiting example, the food corer 200 may be provided with a weigh tension module.

The food corer 200 may be made of plastic materials, including but not limited to, polycarbonate (PC), polystyrene (PS), poly(methyl methacrylate) (PMMA), polyester (PET), polypropylene (PP), high density polyethylene (HDPE), polyvinylchloride (PVC), thermoplastic elastomer (TPE), thermoplastic urethane (TPU), acetal (POM), polytetrafluoroethylene (PTFE), or any polymer, and combinations thereof.

In some embodiments, the sampler may be further configured to be user friendly. For example, the handle 220 may comprise a textured surface to create better visual and tactile differentiation between the grip area and sample areas, communicating the user where to hold the sampler 200.

The sampler (e.g., the corer 200) may be operatively associated with an analytical cartridge (e.g., the disposable cup 300) and/or a detection device (e.g., the device 100). Optionally, the sampler may comprise an interface for connecting to the cartridge. Optionally, a cap may be positioned on the proximal end of the sampler. The sampler 200 may also comprise a sensor positioned with the sampler 200 to detect a presence of a sample in the sampler.

### Disposable Analytical Cartridge

In some embodiments, the present disclosure provides an analytical cartridge or vessel. As used herein, the terms “cartridge”, “vessel” and “test cup” are used interchangeably. The analytical cartridge is constructed for implementing a detection test. As used herein, the analytical cartridge is also referred to as an analytic module. The analytical cartridge is disposable and used for one particular allergen or a particular set of allergens (e.g., a set of tree nuts allergens). A disposable analytical cartridge is constructed for processing a test sample to a state permitting the allergen(s) of interest to engage in an interaction with a detection agent, for example, dissociation of food samples and allergen protein extraction, filtration of food particles, storage of reaction solutions/reagents and detection agents, capture of an allergen of interest using detection agents such as antibodies and nucleic acid molecules that specifically bind to allergen proteins. In one aspect, the detection agents are nucleic acid molecules such as aptamers and/or aptamer derived SPNs. In other embodiments, the detection agents may be antibodies specific to allergen proteins, such as antibodies specific to peanut allergen proteins Ara HI. In other embodiments, the detection agents may be any agents, e.g., chemical compounds, peptide aptamers and complexes that can specifically recognize allergen proteins. The present disclosure discusses food allergens as examples of molecules of interest that can be detected with the present assemblies. One skilled in the art would understand, any targets (i.e., molecules of interest) in a sample can be detected.

In one preferred embodiment, the detection agent is an aptamer, or a derivative thereof, that can specifically bind to an allergen. Aptamers, due to their small size, strong target affinity, lack of immunogenicity, and ease of chemical modification, have emerged as attractive alternatives to other molecular detection technologies, such as antibodies. Aptamers are oligonucleotides capable of high-affinity binding to specific target molecules. Since the development of the in vitro selection process, the systematic evolution of ligands by exponential enrichment (SELEX) in 1990, aptamers have been designed to selectively bind diverse targets, including RNA, DNA, small molecules and compounds and have gained traction as valuable tools for fundamental research, therapeutic applications, and as sensors in molecular diagnostic devices. They have also gained traction in several clinical applications, and the first aptamer based therapeutic was FDA approved in 2004 for the treatment of age-related macular degeneration.

Furthermore, aptamers are chemically synthesized with accuracy and can be stored for long periods of time post-synthesis. They can also be reproducibly modified with fluorophores or nucleic acid analogues. Due to the intensive SELEX process, aptamers can exhibit high affinity and specificity comparable to monoclonal antibodies. They also have been proven for the recognition of diverse antigens, from DNA, RNA, proteins, and cells.

In some embodiments, the detection agent is an aptamer that comprises a nucleic acid sequence that specifically binds to an allergen, or a signaling polynucleotide (SPN) derived from an allergen specific aptamer. For example, the SPN may comprise a core sequence specific to an allergen and may be labeled with a fluorophore at one end of the sequence.

In accordance with the present disclosure, at least one separate analytical cartridge is provided as part of the assembly. In other embodiments, the analytical cartridge may be constructed for use with any other detection systems.

In some embodiments, a disposable analytical cartridge is intended to be used only once for an allergen test in a sample and therefore may be made of low cost plastic materials, for example, acrylonitrile butadiene styrene (ABS), COC (cyclic olefin copolymer), COP (cyclo-olefin polymer), transparent high density polyethylene (HDPE), polycarbonate (PC), poly(methyl methacrylate) (PMMA), polypropylene (PP), polyvinylchloride (PVC), polystyrene (PS), polyester (PET), or other thermoplastics. Accordingly, a disposable analytical cartridge may be constructed for any particular allergen of interest. In some embodiments, these disposable cartridges may be constructed for one particular allergen only, which may avoid cross contamination with other allergen reactions.

In some embodiments, the disposable cartridge is made of polypropylene (PP), COC (cyclic olefin copolymer). COP (cyclo-olefin polymer), PMMA (poly(methyl methacrylate), or acrylonitrile butadiene styrene (ABS).

In other embodiments, these analytical cartridges may be constructed for detecting two or more different allergens in a test sample in parallel. In some aspects, the cartridges may be constructed for detecting two, three, four, five, six, seven, or eight different allergens in parallel. In one aspect, the presence of multiple allergens, e.g., two, three, four, five, or more, are detected simultaneously, a positive signal may be generated indicating which allergen is present. In another aspect, a system is provided to detect if an allergen, e.g., peanut or a tree-nut, is present and generate a signal to indicate the presence of such allergen.

In some embodiments, the disposable analytical cartridge may further be constructed to comprise a bar code that can store the lot specific parameters. The stored information may be later read and stored in any digital formats by the user.

In some embodiments, the analytical cartridge comprises a homogenizer configured to produce a homogenized sample, thereby releasing the molecule of interest from a matrix of the sample into an extraction buffer that optionally includes the detection agent. The analytical cartridge also comprises a first conduit to transfer the homogenized sample with or without the detection agent through a filter system to provide a filtrate containing the molecule of interest and the detection agent and a second conduit to transfer the filtrate, making the filtrate to be contacted with a detection probe, thereby permitting an interaction of the detection agent with the detection probe. The first and second conduits comprise a plurality of fluidic paths connecting different parts of the conduits from transferring the processed sample, buffers, filtrate, waste, and other fluids.

In some embodiments, the analytical cartridge may further comprise a rotary valve system providing a mechanism for controlling the transfer of the sample and other fluidic components (e.g., buffer, filtrate, reaction mixture, reagents and waste) in the analytical cartridge. The valve may also measure and control the volume of fluidic components moving in different compartments of the cartridge. The rotary valve switching system may be further configured to provide a closed position to prevent fluid movement in the analytical cartridge.

In some embodiments, the homogenizer and the rotary valve system may be powered by motors located in the detector unit when the analytical cartridge is accepted by the detector unit, or any other motor mechanisms provided by a connected detection device.

In some embodiments, the analytical cartridge may be constructed to comprise one or more separate chambers, each configured for separate functions such as sample reception, protein extraction, filtration, storage for buffers, agents and waste solution, and detection reaction. The chambers are separate but connected for operation. For example, the analytical cartridge may include a sample processing chamber, a detection chamber, a waste chamber, and optionally a buffer chamber. In some embodiments, the analytical cartridge may further comprise a separate filtrate chamber to hold the filtrate and optionally further concentrate the filtrate prior to the transfer to the detection chamber. In some examples, the detection chamber may comprise a detection sensor and an optical window. The detection mechanism of the detector unit analyzes the detection reaction through the optical window to identify the interaction of the molecule of interest with the detection agent in the detection chamber. The detection window is operatively associated with the detection mechanism of a detection device.

In some embodiments, the analytical cartridge comprises a detection sensor for measuring the interaction between the target molecule and the detection agent. The detection sensor is included in the detection chamber. In one non-limiting example, the detection sensor is a transparent substrate which includes a plurality of fluidic channels and a detector chip area. The substrate is referred to as a chipannel, wherein the fluidic channels and the detector chip area are connected. In some examples, the chipannel is a plastic substrate.

In some embodiments, the detector chip area within the chipannel comprises at least one reaction panel and at least one control panel. In other embodiments, the detector chip area within the chipannel may comprise one reaction panel and two control panels. In other embodiments, the chipannel may comprise a plurality of reaction panels and a plurality of control panels. Optionally, the detector chip area of the chipannel further comprises one or more fiducial spots that guide image processing by an imaging mechanism (e.g., a camera) of the detector unit. Any suitable fiducial object may be spotted as a fiducial marker for reference.

In some embodiments, the chipannel comprises a detection probe molecule immobilized on the reaction panel of the detector chip area. The detection probe is configured to engage in a probe interaction with the detection agent. An interaction of the molecule of interest with the detection agent prevents the detection agent from engaging in the probe interaction with the detection probe. The detector chip area within the chipannel may further include an optically detectable control probe molecule immobilized on the control panel(s), for normalization of signal output measured by the detection mechanism. In some embodiments, the control probe molecule is a nucleic acid molecule that does not bind to the molecule of interest or the detection agent.

In one preferred embodiment, the chipannel is a plastic chip wherein the reaction panel is printed with a nucleic acid-based detection probe that comprises a nucleic acid sequence complementary to nucleic acid sequence of the detection agent and wherein the control panel is printed with nucleic acid based control probe molecule that does not bind to the detection agent.

In some embodiments, detection agents, detection probes, buffers such as extraction buffers and wash buffers, and other components necessary for assembling a functional cartridge are included.

In some embodiments, the analytical cartridge may comprise a data chip unit configured for providing the cartridge information.

In accordance with the present disclosure, the analytical cartridge may be construed in any suitable shape and size. Some exemplary embodiments of the analytical cartridge are illustrated below. The exemplary embodiments do not intent to limit the design of the cartridge.

In some embodiments, homogenization buffer with magnesium chloride (MgCl₂) is filled into the filtrate chamber of the analytic cartridge. The concentration of MgCl₂ ranges from 10 mM to 100 mM, or from 10 mM to 80 mM, or from 10 mM to 60 mM, or from 10 mM to 40 mM, or from 20 mM to 100 mM, or from 20 to 80 mM, or from 20 mM to 50 mM. In other embodiments, the concentration of MgCl₂ ranges from 1 mM to 10 mM, or from 1 mM to 5 mM, or from 2 mM to 10 mM, or from 2 mM to 8 mM, or from 5 mM to 10 mM.

In some embodiments, the reaction chamber is washed once, twice or three times before reading the reaction signal. The wash buffer may contain magnesium chloride at a concentration from 0.1 mM to 1.0 mM, such as 0.1 mM, 0.25 mM, 0.75 mM, and 1.0 mM.

### Exemplary Embodiments of the Analytical Cartridge

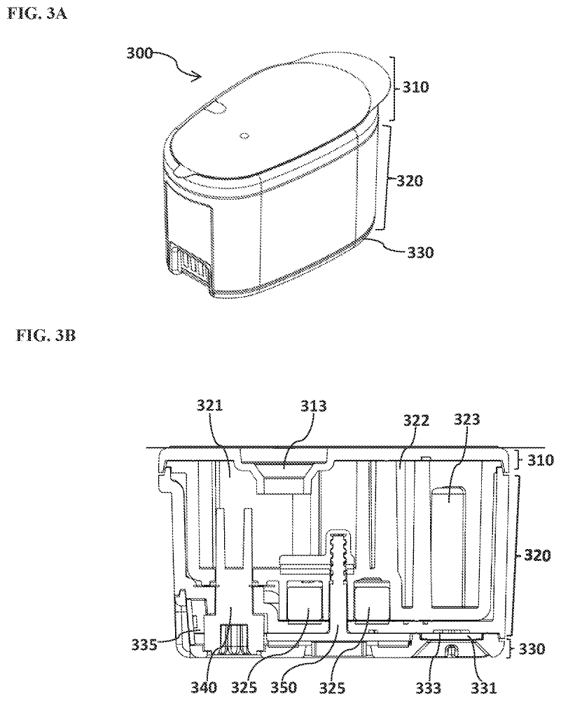

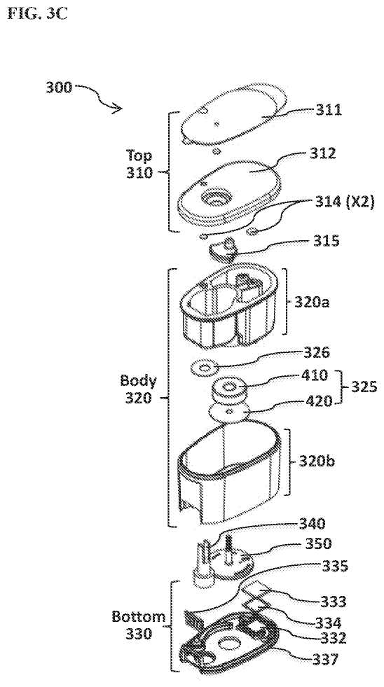

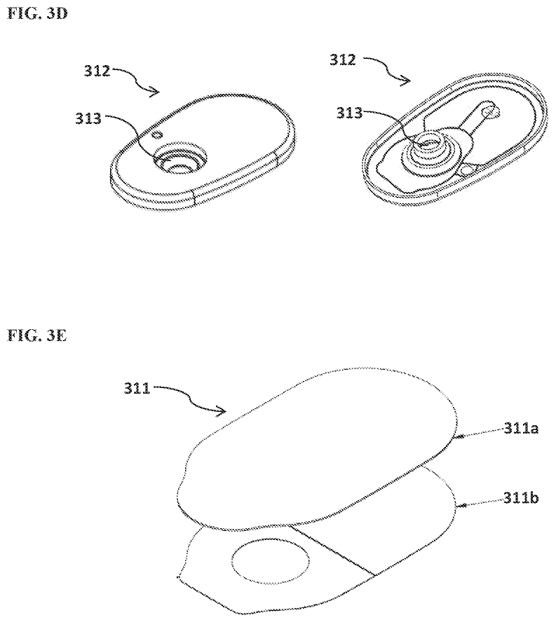

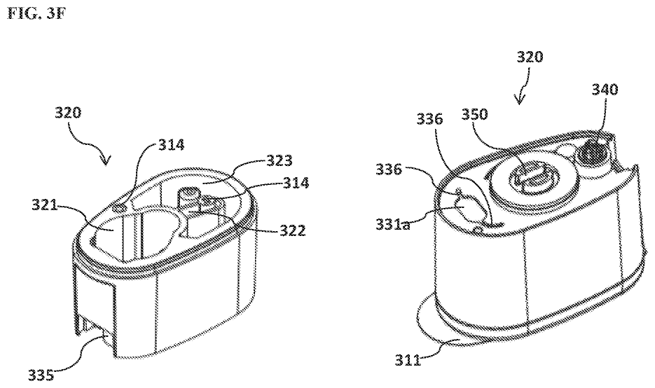

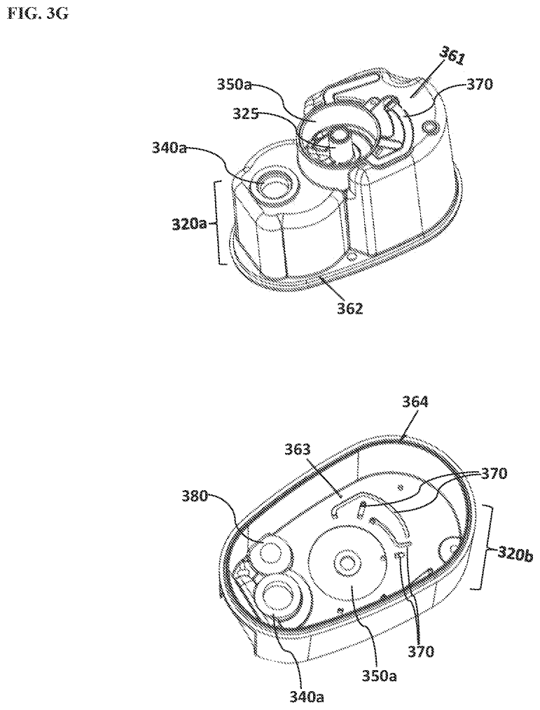

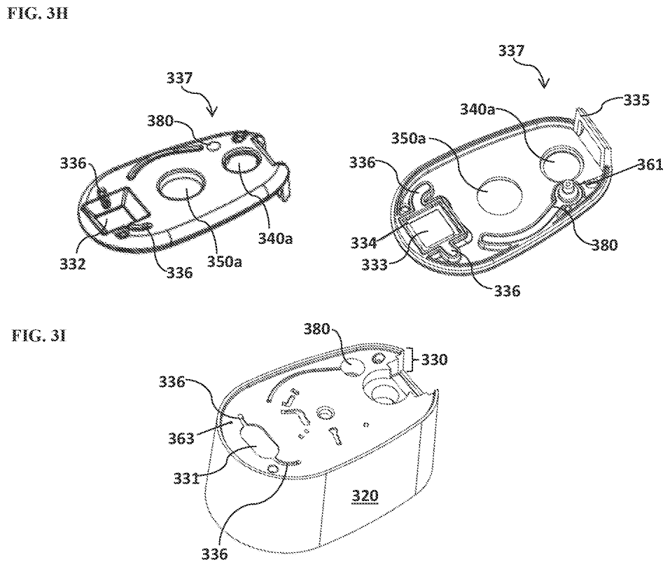

In some embodiments, the disposable analytical cartridge may be construed as a disposable test cup or a cup-like container. The cup container may comprise several compartments that are assembled into a functional analytic module. According to one embodiment of the test cup, as shown in FIG. 3A, the assembled disposable test cup 300 comprises three parts: a cup top 310, a cup body 320 and a cup bottom 330. The three parts are operatively connected to assemble a functional analytical module. The cup 300 further comprises a homogenization rotor 340 that rotates in both directions to homogenize the sample, a filter assembly 325 filtrating the processed sample, a rotary valve 350 contemplated to control the fluid flow inside the cup (FIG. 3B), and fluidic paths transporting the processed sample, mixer, filtrate, buffers and agents to different compartments of the test cup (not shown in FIG. 3B).

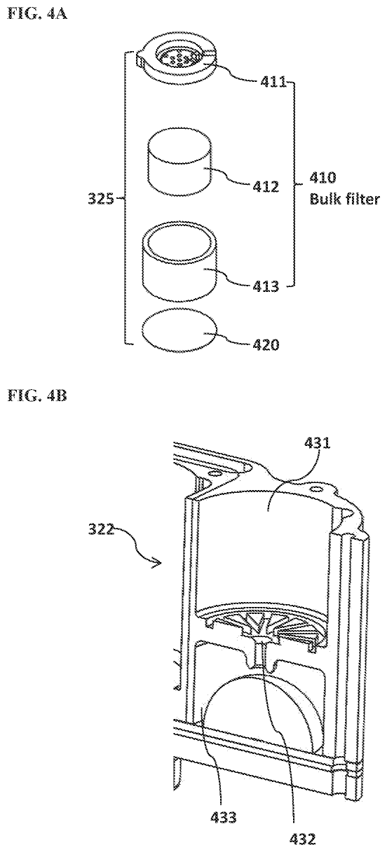

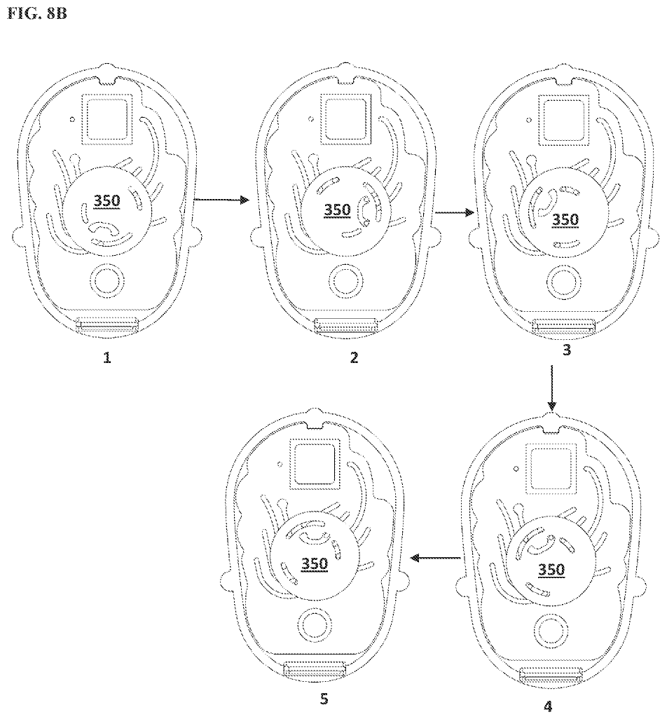

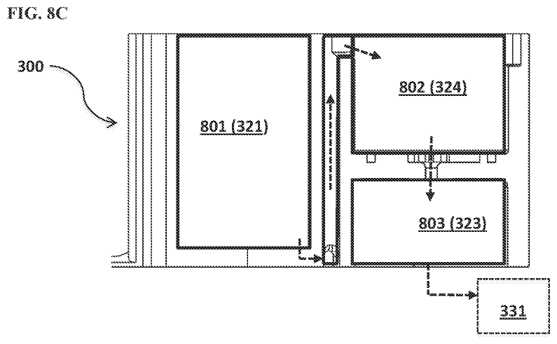

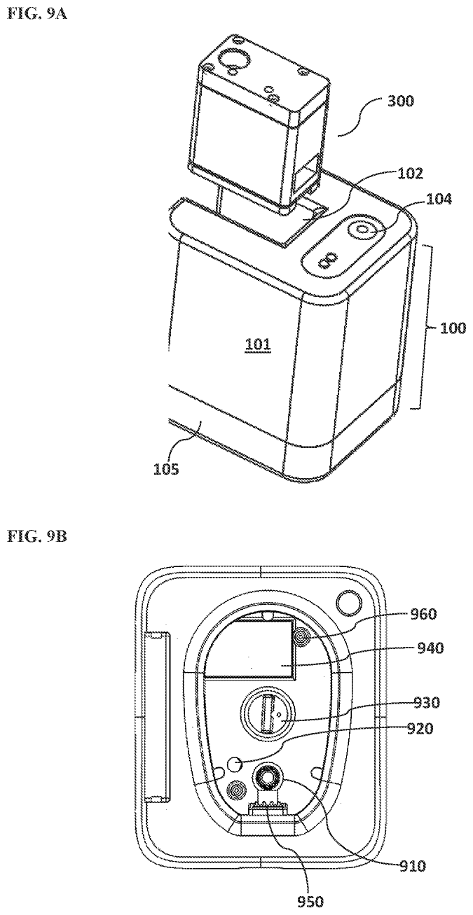

The test cup body 320 may include a plurality of chambers. In one embodiment, as shown in FIG. 3B, the test cup body 320 includes one homogenization chamber 321 comprising a food processing reservoir 801 (as shown in FIG. 8C), a filtrate chamber 322 for collecting a sample solution after being filtered through the filter (e.g., the 2-state filter 325 shown in FIG. 3B and FIG. 4A), a waste chamber 323 comprising a waste reservoir 803 (as shown in FIG. 8C), and optionally, a wash buffer storage chamber 324 comprising wash buffer storage reservoir 802 (as shown in FIG. 8C). Optionally, one or more separate wash compartments may be included in the cup body 320. In some embodiments, a reaction chamber 331 at the cup bottom 330 for receiving the processed sample (also referred to herein as a signal detection chamber) is included shown in FIGS. 3B and 3H. The reaction/detection chamber 331 may comprise a separate detection sensor (e.g., the chip 333 shown in FIG. 3B) with a detection probe that reacts with the processed sample. All analytical reactions occur in the reaction/detection chamber 331, and a detectable signal (e.g., a fluorescence signal) is generated therein. In some embodiments, detection agents (e.g., SPNs) for example, which are pre-stored in the homogenization chamber 321, may be premixed with the test sample in the homogenization chamber 321, where the test sample is homogenized and the extracted allergen proteins react with the detection agents. The mixed reaction complexes may be transported to the filter 325 before they are transported to the reaction/detection chamber 331. In other examples, detection agents (e.g., SPNs) may be stored in the filtrate chamber 322. The processed sample is filtered through the filter assembly 325 and reacts with the detection agents stored in the filtrate chamber 322. The filtrate containing the molecule of interest and detection agents is transferred to the detection chamber 331 wherein the detection agents engage an interaction with the detection probes immobilized on the sensor (e.g., the chip 333) and the detection signal is measured.

In alternative embodiments, more than one buffer and reagent storage reservoir may be included in the buffer and reagent storage chamber 324. As a non-limiting example, the extraction buffer and wash buffers may be stored separately in a reservoir within the buffer storage chamber 324.

FIG. 3C shows an exploded view of one exemplary embodiment of the disposable test cup 300 which is configured to contain three main components, the top 310, the housing or body 320 and the bottom 330. The cup top 310 may include a cup lid 311, a top cover 312, two or more breather filters 314 which are included to ensure that only air is brought in and that fluids do not escape from the test cup 300. The cup body 320 is composed of two separate parts: an upper housing 320a and an outer housing 320b. The cup bottom assembly 330 includes a bottom cover 337 that sandwiches other components including the reaction chamber 331 (in FIGS. 3F and 3H), a detection sensor, i.e., a glass chip 333, and a chip gasket 334 that facilitates the attachment of the glass chip 333 to the bottom of the specialized sensor area 332 in the reaction chamber 331. In some embodiments, the processed sample mixer flows to the reaction chamber 331 and reacts with the detection agents on the chip 333 to generate detectable signals. For example, the chip 333 may be coated with oligonucleotide sequences to detect targets presented in the test sample. The bottom cover 337 also comprise a port/bit 340a for holding the homogenization rotor 340 and a port/bit 350a for holding the rotary valve 350 (as shown in FIG. 3H). These bits provide a means for linking the homogenization rotor 340 and the rotary valve 350 to the motors of the detection device 100. In some embodiments, a rotor gasket 326 may be configured to the upper housing 320a to seal the rotor 340 to the housing 320, to avoid leakage of fluids. In some embodiments, the bottom cover may further comprise fluidic paths and air channels.

In some embodiments, the cup may further be constructed to comprise a bar code that can store the lot specific parameters. In one example, the bar code may be the data chip 335 that stores the cup 300 specific parameters, including the information of detection agents such as SPNs (e.g., fluorophore labels, the target allergen, and intensity of SPNs, etc.), expiration date, manufacture information, etc.

FIG. 3D further demonstrates the features of the top cover 312 of the cup shown in FIG. 3A. A corer port 313 is included for receiving a food corer 200, thereby receiving the picked test sample and transferring the sample to the sample processing chamber 321 (also referred to as homogenization chamber). As a non-limiting example, the port 313 may be configured for receiving the food corer 200 shown in FIG. 2B. The top cover 312 may also include at least one small hole (FIG. 3D) for air to be drawn in for fluid flow. As a non-limiting example, the top part may have two lids 311. As discussed hereinabove, the lid 311 may comprise two layers: a top lid 311a for scaling and labeling and a bottom 311b for resealing during operation. As shown in FIG. 3E, the second lid at the bottom 311b is constructed for resealing and liquid retention during the operation. The top lid 311a may be peeled back for inserting the test sample collected by the corer 200, and then reclosed after assay completion.

FIG. 3F is a top view of a cup housing body 320 as the upper housing 320a and the outer housing 320b are assembled together (left panel). The upper housing 320a may comprise one or more chambers which are operatively connected. In one embodiment, the homogenization chamber 321, filtration chamber 322 and waste chamber 323 are included in the housing 320a (left panel). Two breath filters 314 are also added to the upper housing 320a. The bottom of the assembled cup body 320 comprises an opening 331a that connects to the reaction/detection chamber 331 with the inlet and outlet 336 for fluid flow (right panel). In this embodiment, the reaction/detection chamber 331 is formed when the bottom cover 337 is assembled together with the body part (see FIG. 3C) The rotor 340 and the rotary valve 350 may be assembled into the cup to form an analytical cartridge (right panel).

FIG. 3G further illustrates the outer interface of the bottom of the upper housing (320a) (upper panel) and the inner interface of the bottom of the outer housing 320b (lower panel). The two energy director faces 361 (face 1) and 362 (face 2) at the outer interface of the upper housing 320a, interact with the two welding mating faces, face 363 (face 1) and 364 (face 2) at the inner interface of the bottom of the outer housing 320b to retain the housing parts 320a and 320b together to form the cup body 320. Fluid paths 370 are also included to flow liquids to the cup bottom 330. The rotor 340 and the rotary valve 350 are assembled into the cup through the rotor port 340a and the rotary valve port 350a, respectively.

FIG. 3H further illustrates the cup bottom cover 337 of the cup bottom 330 of the cup 300 shown in FIG. 3A and FIG. 3C. The reaction/detection chamber 331 comprises a specialized sensor area 332 where a detection sensor, i.e., the glass chip 333, is positioned through a glass gasket 334. The glass gasket 334 may be included to seal the glass chip 333 in place to the bottom of the reaction chamber 331 and to prevent fluid leakage. Alternatively, adhesive or ultrasonic bonding can be used to mate the layers together. In some aspects of the present disclosure, the glass chip 333 may be configured directly at the bottom of the reaction chamber 331 (e.g., the bottom surface of the sensor area 332) as a component of the cup bottom cover 337, and integrated into the cup body as one entity. The entire unit may be of PMMA (poly(methyl methacrylate)) (also referred to as acrylic or acrylic glass). This transparent PMMA acrylic glass may be used as optic window for signal detection.

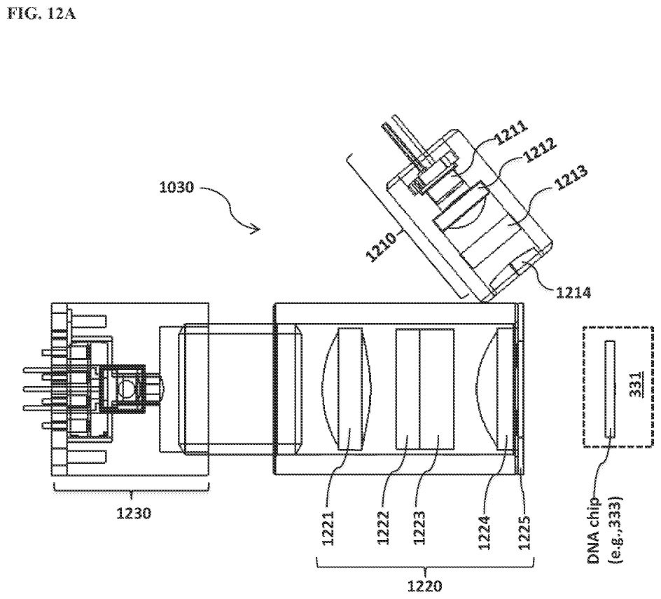

The reaction chamber 331 comprises at least one optical window. In one embodiment, the chamber 331 comprises two optical windows, one primary optical window and one secondary optical window. In some embodiments, the primary optical window serves as the interface of the reaction chamber 331 to the detection device 100, in particular to the optical system 1030 (as shown in FIGS. 10A, 10B, and 12A-12C) of the detection device 100. The detection sensor (e.g., the glass chip 333) may be positioned between the optical window and the interface of the optical system. The optional secondary optical window may locate at one side of the reaction chamber 331; the secondary optical window allows detection of the background signals. In some aspects of the present disclosure, the secondary optical window may be constructed for measuring scattered light.

As shown in FIG. 3I, the bottom 330 is assembled with the cup body 320. From this bottom perspective view, the bottom surface comprises several interfaces for fluid paths (e.g. fluidic inlet/outlet 336) and a plump interface 380 and the interfaces connecting the rotor 340 and the rotary valve 350 to the detection device 100.

A means may be included to the cup to block the fluid flows between the compartments of the assembled cup 300. In one embodiment, a dump valve 315 (shown in FIG. 3C) in the cup housing 320a is included to block fluid in the homogenization chamber 321 from flowing to the rotary valve 350 that is configured at the bottom of the cup 300. The dump valve 315 is held in place by the rotary valve 350 (FIG. 3C) for shipping, storage, and end of life. The rotary valve 350 locks the dump valve 315 over the filters (e.g., the filter assembly 325) during shipping and prevents fluid flow after completing the detection assay. The rotary valve 350 may be actuated in several steps to direct fluid flow to the proper chambers. As a non-limiting example, the relevant positions of the rotary valve 350 during the detection test are demonstrated in FIG. 8B.

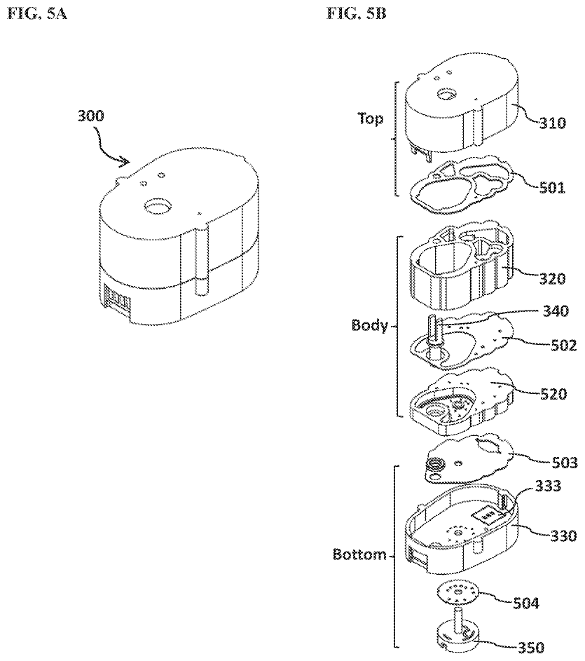

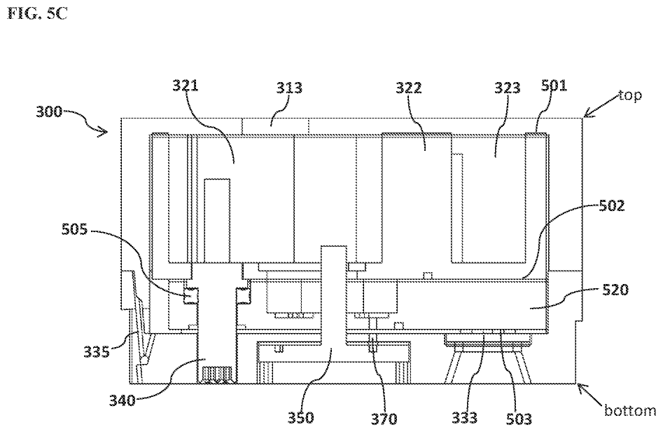

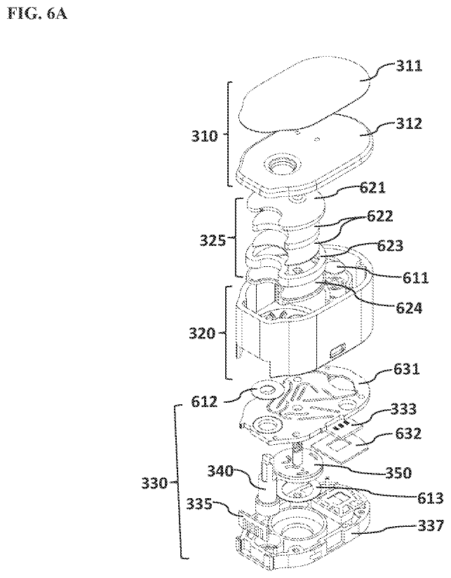

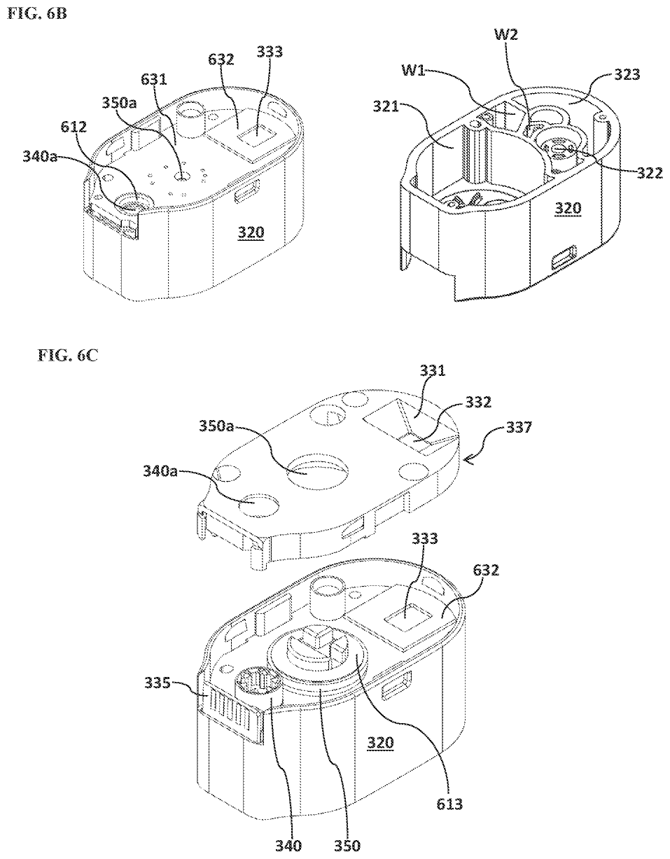

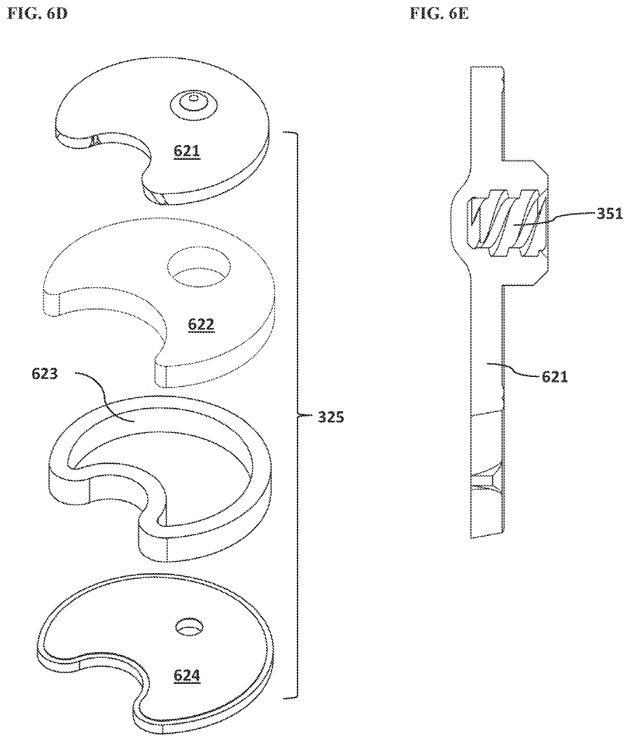

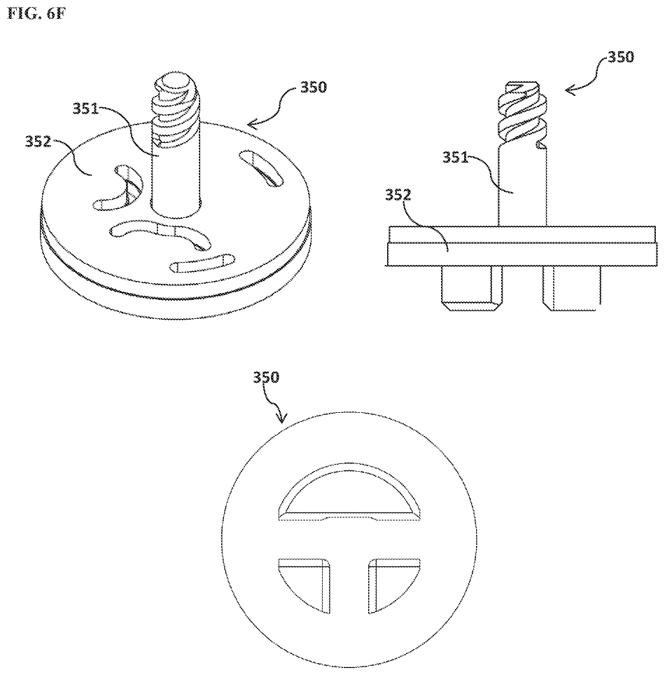

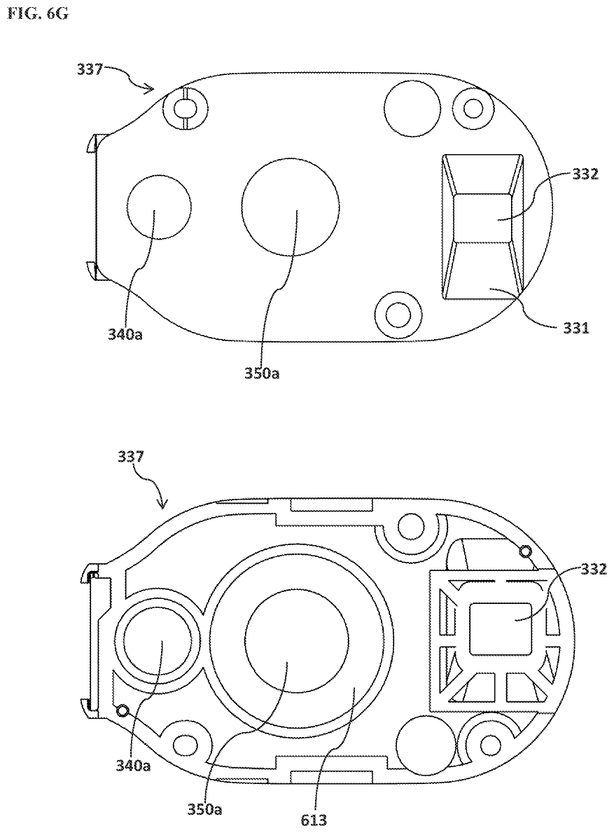

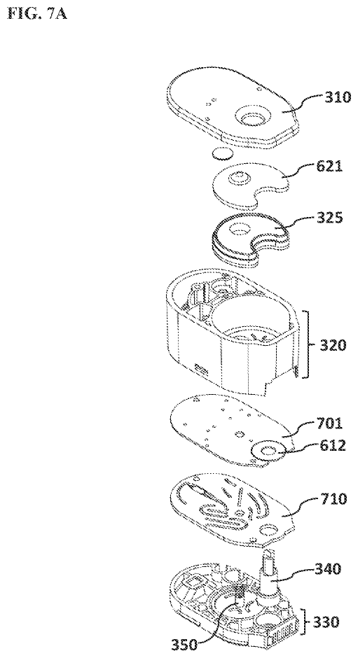

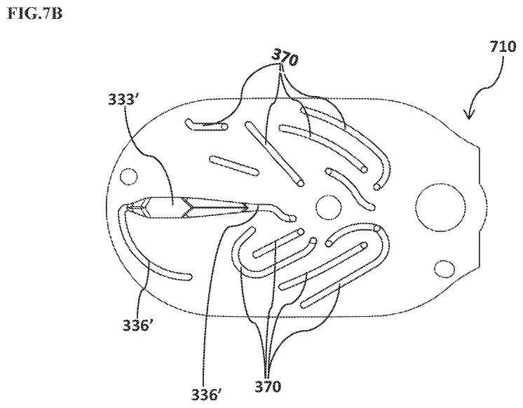

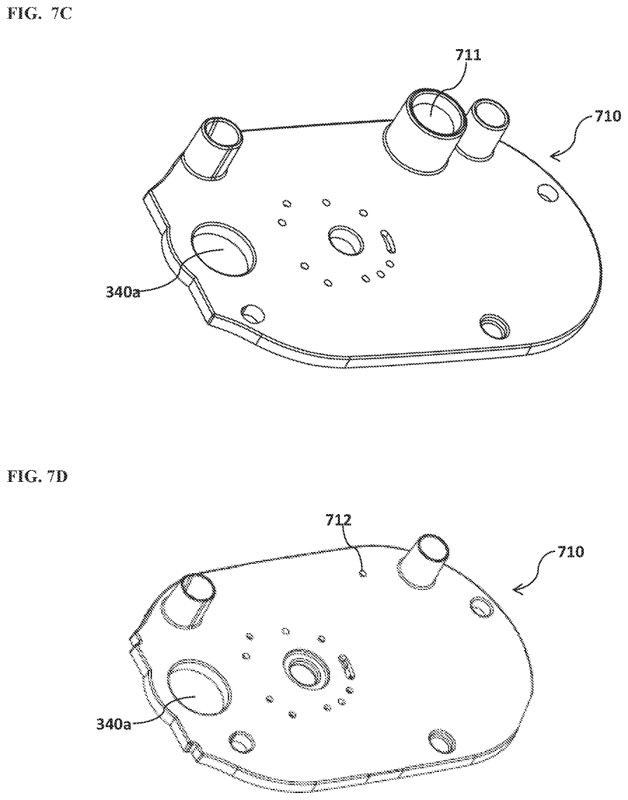

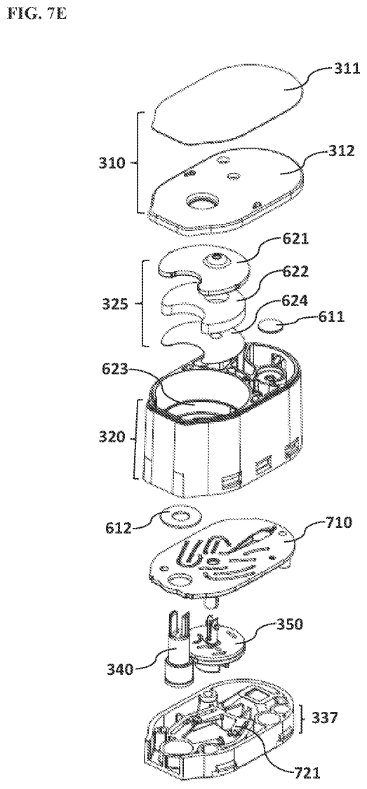

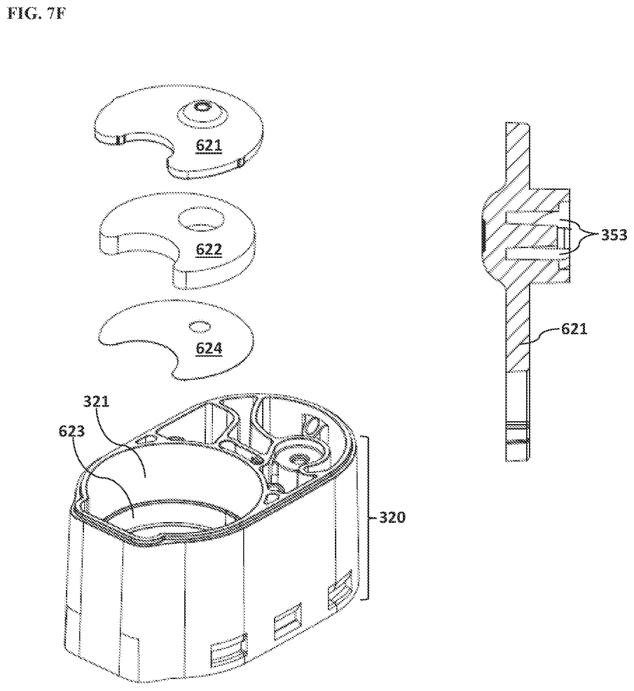

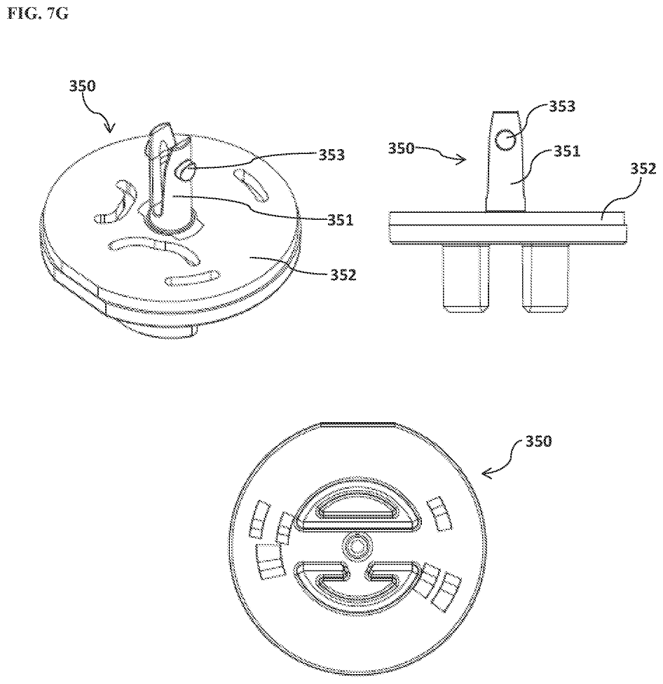

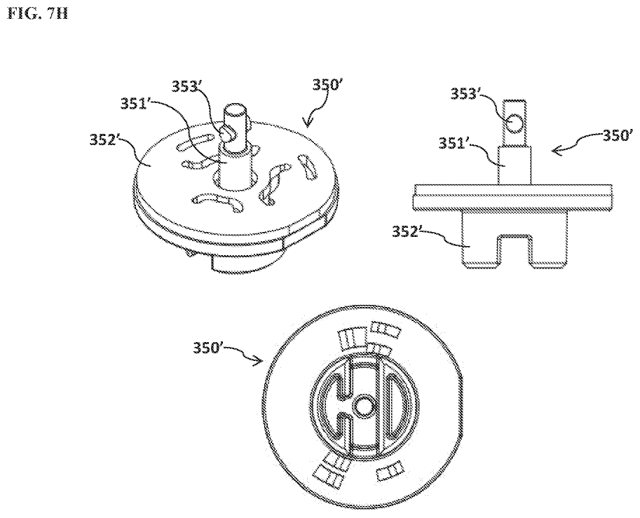

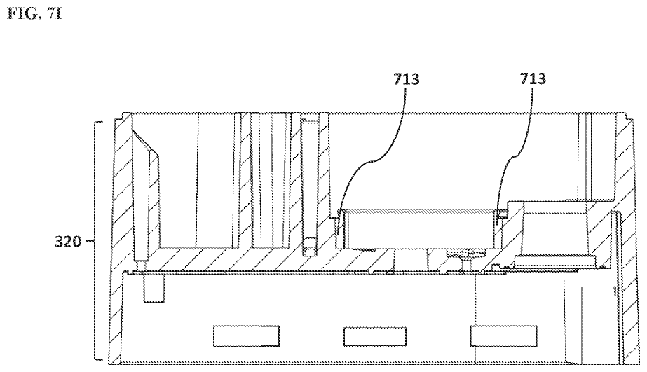

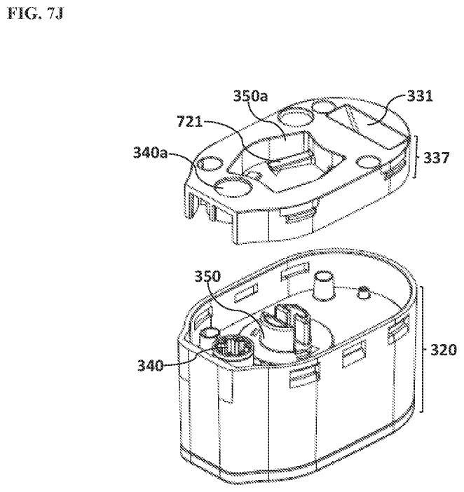

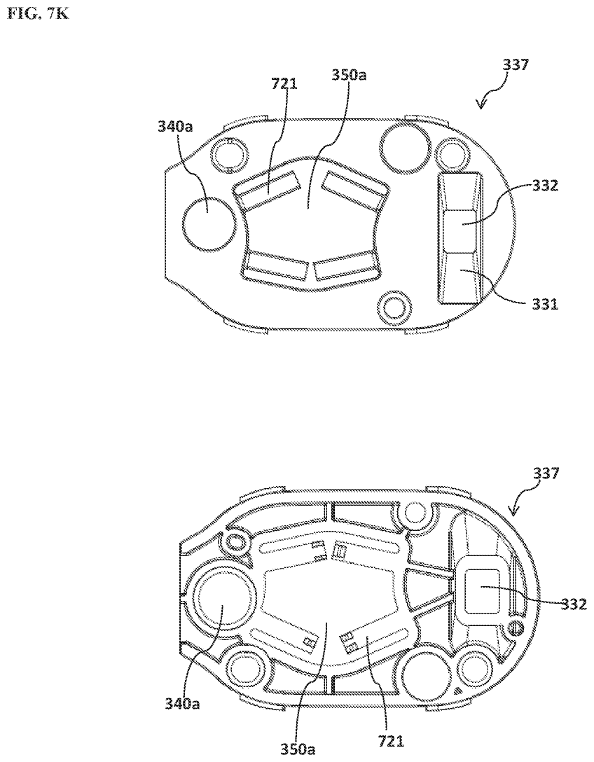

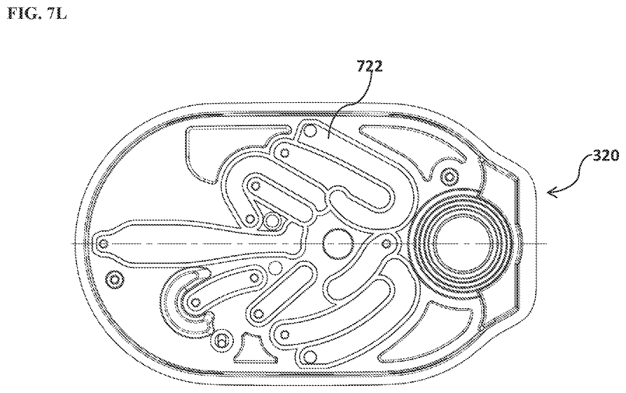

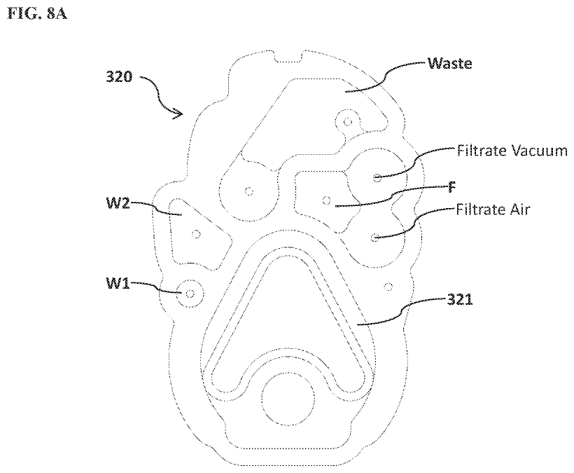

The rotary valve 350 may rotate to regulate the fluid flow through the chambers inside the cartridge. In some embodiments, the rotary valve 350 may comprise a valve shaft 351 that is operatively connected to and locks the dump valve 315 (as shown in FIG. 3C) and a valve disc 352 connected to the valve shaft 351 (e.g., in FIG. 6F). The rotary valve 350 can be attached to the cup through any available means known in the art. In one embodiment, a valve gasket (e.g., the gasket 504 shown in FIG. 5B) may be used. Alternatively, the rotary valve can be attached to the cup through a disc spring (e.g., a wave disc spring). In another embodiment, the rotary valve 350 may be secured to the cup with a plurality of compression coil springs (e.g., 721 shown in FIG. 7K).

In some embodiments, a filter assembly (e.g., the filter 325 shown in FIG. 3C, FIG. 4A and FIG. 6D) is included in the analytical cartridge. The filter removes large particles and other interfering components from the sample, such as fat from a food matrix, before the processed sample is transferred into the reaction chamber 331.

In some embodiments, the filter mechanism may be a filter assembly. The filter assembly may be a simple membrane filter 420. The membrane 420 may be a nylon, PE, PET, PES (poly-ethersulfone), Porex™, glass fiber, or the membrane polymers such as mixed cellulose esters (MCE), cellulose acetate, PTFE, polycarbonate, PCTE (Polycarbonate) or PVDF (polyvinylidene difluoride), or the like. It may be a thin membrane (e.g., 150 μm thick) with high porosity. In some aspects, the pore size of the filter membrane 420 may range from 0.01 μm to 600 μm, or from 0.1 μm to 100 μm, or from 0.1 μm to 50 μm, or from 1 μm to 20 μm, or from 20 μm to 100 μm, or from 20 μm to 300 μm, or 100 μm to 600 μm or any size in between. For example, the pore size may be about 0.02 μm, about 0.05 μm, about 0.1 μm, about 0.2 μm, about 0.5 μm, about 1.0 μm, about 1.5 μm, about 2.0 μm, about 2.5 μm, about 3 μm, about 3.5 μm, about 4.0 μm, about 4.5 μm, about 5.0 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 100 μm, about 150 μm, about 200 μm, about 250 μm, about 300 μm, about 350 μm, about 400 μm, about 450 μm, about 500 μm, about 550 μm, or about 600 μm.

In some alternative embodiments, the filter assembly may be a complex filter assembly 325 (as shown in FIG. 4A) comprising several layers of filter materials. In one example, the filter assembly 325 may comprise a bulk filter 410 composed of a gross filter 411, a depth filter 412, and a membrane filter 420 (FIG. 4A). In one embodiment, the gross filter 411 and the depth filter 412 may be held by a retainer ring 413 to form a bulk filter 410 sitting on the membrane filter 420. In other embodiments, the bulk filter 410 may further comprise a powder that sits inside the filter or on top of the filter. The powder may be selected from cellulose, PVPP, resin, or the like. In some examples, the powder does not bind to nucleic acids and proteins.

In some embodiments, the filter assembly 325 may be optimized for removing oils from highly fatty samples, but not proteins and nucleic acids, resulting in superior sample cleaning. In other embodiments, the ratio of the depth and width of the filter assembly 325 may be optimized to maximize the filtration efficiency.

In some embodiments, the filter assembly 325 may be placed inside a filter bed chamber 431 in the disposable cup body 320. The filter bed chamber 431 may be connected to the homogenization chamber 321. The homogenate can be fed to the filter assembly 325 inside the filter bed chamber 431. The filtrate is collected by the collection gutter 432 (also referred to herein as filtrate chamber) (FIG. 4B). The collected filtrate then can exit the fluidics to flow to the reaction chamber 331 (FIG. 3B). In one example, the collected filtrate may be transported to the reaction chamber 331 from the collection gutter 432 directly. In another example, the filtrate may be first transported to the filtrate collection chamber 433 before being transported to the reaction chamber 331 through the inlet/outlet 336 (FIG. 3H). The fluids may be delivered to the reaction chamber 331 by the fluid paths 370 at the bottom of the cup 320 (as shown in FIG. 3G).

In some embodiments, the filtrate collection chamber 433 may further comprise a filtrate concentrator which is configured to concentrate the sample filtrate before it flows to the reaction chamber 331 for signal detection. The concentrator may be in a half-ball shape, or a conical type concentrator, or a tall pipe.

In accordance, the processed sample (e.g., the homogenate from the chamber 321) is filtered sequentially through the gross filter 411, the depth filter 412 and the membrane filter 420. The gross filter 411 can filter a large particle suspension from the sample, for example, particles larger than 1 mm, and/or some dyes. The depth filter 412 may remove small particle collections and oil components from the sample (such as the food sample). The pore size of the depth filter 412 may range from about 1 μm to about 500 μm, or about 1 μm to about 100 μm, or about 1 μm to about 50 μm, or about 1 μm to about 20 μm, or about 4 μm to about 20 μm, or from about 4 μm to about 15 μm. For example, the pore size of the depth filter 412 may be about 2 μm, or about 3 μm, or about 4 μm, or about 5 μm, or about 6 μm, or about 7 μm, or about 8 μm, or about 9 μm, or about 10 μm, or about 11 μm, or about 12 μm, or about 13 μm, or about 14 μm, or about 15 μm, or about 16 μm, or about 17 μm, or about 18 μm, or about 19 μm, or about 20 μm, or about 25 μm, or about 30 μm, or about 35 μm, or about 40 μm, or about 45 μm, or about 50 μm.

The depth filter 412 may be composed of, for example, cotton including, but not limited to raw cotton and bleached cotton, polyester mesh (monofilament polyester fiber) and sand (silica). In some embodiments, the filter material may be hydrophobic, hydrophilic or oleophobic. In some examples, the material does not bind to nucleic acids and proteins. In one embodiment, the depth filter is a cotton depth filter. The cotton depth filter may vary in sizes. For example, the cotton depth filter may have a ratio of width and height ranging from about 1:5 to about 1:20. The cotton depth filter 412 may be configured to correlate total filter volume and the food mass being filtered.

The membrane filter 420 can remove small particles less than 10 μm in size, or less than 5 μm in size, or less than 1 μm in size. The pore size of the membrane may range from about 0.001 μm to about 20 μm, or from 0.01 μm to about 10 μm. Preferably the pore size of the filter membrane may be about 0.001 μm, or about 0.01, or about 0.015 μm, or about 0.02 μm, or about 0.025 μm, or about 0.03 μm, or about 0.035 μm, or about 0.04 μm, or about 0.045 μm, or about 0.05 μm, or about 0.055 μm, or about 0.06 μm, or about 0.065 μm, or about 0.07 μm, or about 0.075 μm, or about 0.08 μm, or about 0.085 μm, or about 0.09 μm, or about 0.095 μm, or about 0.1 μm, or about 0.15 μm, or about 0.2 μm, or about 0.2 μm, or about 0.25 μm, or about 0.3 μm, or about 0.35 μm, or about 0.4 μm, or about 0.45 μm, or about 0.5 μm, or about 0.55 μm, or about 0.6 μm, or about 0.65 μm, or about 0.7 μm, or about 0.75 μm, or about 0.8 μm, or about 0.85 μm, or about 0.9 μm, or about 1.0 μm, or about 1.5 μm, or about 2.0 μm, or about 3.0 μm, or about 3.5 μm, or about 4.0 μm, or about 4.5 μm, or about 5.0 μm, or about 6.0 μm, or about 7.0 μm, or about 8.0 μm, or about 9.0 μm, or about 10 μm. As discussed above, the membrane may be a nylon membrane, PE, PET, a PES (poly-ethersulfone) membrane, a glass fiber membrane, a polymer membrane such as mixed cellulose esters (MCE) membrane, cellulose acetate membrane, cellulose nitrate membrane, PTFE membrane, polycarbonate membrane, Track-Etched polycarbonate membrane, PCTE (Polycarbonate) membrane, polypropylene membrane, PVDF (polyvinylidene difluoride) membrane, or nylon and polyamide membrane.

In one embodiment, the membrane filter is a PET membrane filter with 1 μm pore size. The small pore size can prevent particles larger than 1 μm to pass into the reaction chamber. In another embodiment, the filter assembly may comprise a cotton filter combined with a PET mesh having 1 μm pore size.

In other embodiments, the filter components may be assembled together by any known methods in the art, such as by heat welding, ultrasonic welding or a similar process that ensures the assembled materials can be die-cut and packaged without damaging or inhibiting the performance of each filter independently or as an integrated filter assembly. In other embodiments, the packaging of each part the filter assembly enables high-speed automation systems on a manufacturing assembly line (e.g., a robotic assembly line).

In some embodiments, the filtration mechanism has low protein binding, low or no nucleic acid binding. The filter may act as a bulk filter to remove fat and emulsifiers and large particles, resulting in a filtrate with comparable viscosity to the buffer.

In some embodiments, the filter assembly 325 including the gross filter 411, the depth filter 412 and the membrane filter 420 can allow the maximal recovery of signaling polynucleotides (SPNs) and other detection agents.

In other embodiments, the filtration assembly 325 may be configured to comprise a filter 624 (e.g., a mesh filter) that is inserted to a filter gasket 623, a bulk filter 622 composed of a gross filter and a depth filter and a filter cap 621 (as shown in FIG. 6D). In an alternative embodiment, the filter gasket 623 can be molded into the cup body as an overmolded component of the cup body 320, e.g., in the homogenization chamber 321 (as shown in FIGS. 7E and 7F). The filter 624, the bulk filter 622 and the filter cap 621 are inserted to the overmolded gasket to form a functional filter assembly 325.

In some embodiments, the filtration mechanism can complete the filtering process in less than 1 minute, preferably in about 30 seconds. In one example, the filtration mechanism may be able to collect the sample within 35 seconds, or 30 seconds, or 25 seconds, or 20 seconds with less than 10 psi pressure. In some embodiments, the pressure may be less than 9 psi, or less than 8 psi, or less than 7 psi, or less than 6 psi, or less than 5 psi.

In some alternative embodiments, the filtration chamber 322 may comprise one or more additional chambers conjured for filtering the processed sample. As illustrated in FIG. 4B, the filtration chamber 322 may further comprise a separate filter bed chamber 431 wherein a filter assembly 325 (as illustrated in FIG. 4A) is inserted and connected to a collection gutter 432. The collection gutter 432 is configured to collect the filtrate that runs through the filter assembly 325, and the gutter 432 may be directly connected to the flow cell fluidics to flow the filtrate to the reaction chamber 331 for signal detection. Optionally, another collection/concentration chamber 433 may be included in the filtration chamber 322 which is configured for collecting and/or concentrating the filtrate collected through the collection gutter 432 before the filtrate is transported to the reaction chamber 331 for signal detection. The collection/concentration chamber 433 is collected to the filter bed chamber 431 through the collection gutter 432.

FIGS. 5A to 5C illustrate another embodiment of the analytical cartridge. FIG. 5A illustrates an alternative assembly of the test cup 300. The components of the cup 300 of this embodiment are shown in FIG. 5B. According to this embodiment, the cup 300 comprises three parts, a cup top including a cup top cover 310, a cup body comprising a cup tank 320, and a cup bottom including a cup bottom cover 330, which are operatively connected to form an analytic module. As illustrated in FIG. 5B, the top of the cup is a top cover 310 for sealing the cup where a test sample is placed into the cup for testing. A top gasket 501 may be included to seal the top 310 to the cup body 320. The upper cup body 320 comprises the homogenization chamber, waste chamber, chambers for wash buffers (e.g., wash 1 chamber (W1), wash 2 chamber (W2) (shown in FIG. 6B, right panel), and air vent stacks for controlling air and thus fluid flow. A rotor 340 is configured in the homogenization chamber for homogenizing the test sample in an extraction buffer. The shape of the rotor may be adjusted to fit the cup during the assembly. A mid gasket 502 is located at the bottom of the upper cup body 320 to seal the body 320 to the manifold 520 with holes for fluid flow. The manifold 520 is configured to hold the filter 325 and the fluid paths 370 for fluid flow. Another mid gasket 503 is added to seal the manifold 520 to the cup bottom 330, where the reaction chamber (e.g., chamber 331), the detection sensor (e.g., glass chip 333), glass gasket (e.g., gasket 334) and the memory chip (e.g. EPROM) are located. The rotor 340 is sealed to the bottom through an O-ring 505 (shown in FIG. 5C). The rotary valve 350 is configured to the cup 300 at the bottom 330 through a valve gasket 504. In another embodiment, the rotary valve 350 can be configured to the cup 300 through a spring arm, such as wave disc springs and compression coil springs at the cup bottom 330 (e.g., 721 shown in FIG. 7K). The configuration of each components of the cup in FIG. 5A is also illustrated in a section view in FIG. 5C.

According to the present disclosure, a third embodiment of the disposable cup 300 is illustrated in FIG. 6A. FIGS. 6B-6G further illustrate the components of the disposable cup 300 in FIG. 6A. In this embodiment, the configurations of the detection sensor and fluidic paths are further integrated. As shown in FIG. 6A, the cartridge comprises a top part 310, a body part 320 and a bottom part 330. The rotor 340 is sealed to the cup body 320 through a gasket 612. The rotary valve 350 is assembled to the cartridge through a disc spring 613, or alternatively through compression coil springs at the cup bottom part 330 (e.g., 721 shown in FIG. 7K). When implementing a detection assay, the rotary valve 350 may rotate and move the seal 612 to free the rotor 340 for homogenizing the test sample. In this embodiment, a separate panel 631 is provided between the bottom of the cup body 320 and the bottom cover 337 in which the fluidic channels are included. This separate panel 631 with fluidic channels functions equivalently as the fluidic paths 370 of the previous cup embodiments (e.g., FIGS. 3C, 3G and 3I). The sensor chip 333 may be operatively connected to the fluidic panel 631 and the sensor area 332 of the reaction chamber 331 in the bottom cover 337 through a chip PSA 632. In an alternative embodiment, the sensor chip 333 and the fluidic panel 631 may be combined to form a single thin panel (also referred to as a chipannel), therefore forming a separate chipannel 710 (as shown in FIG. 7A). The chipannel 710 is discussed in detail below.

The cup top 310 may comprise a top lid 311 having two labels 311a and 311b as shown in FIG. 3E, and a top cover 312 as shown in FIG. 3D. The cup body 320 may be configured for comprising several separate chambers, including a homogenization chamber 321, a filtration chamber 322, a waste chamber 323, two or more washing spaces (W1 and W2) as shown in FIG. 6B (right panel). In some examples, the filtration chamber 322 has a vent 611 (shown in FIG. 6A). The wetting of the vent 611 can signal to the pressure sensor of the electronics that the chamber 322 is full (FIG. 6B). Similar to other designs, at the bottom of the cup body 320 (FIG. 6B, left panel), several ports are designed including a port 340a for the rotor 340 and a port 350a for the rotary valve 350 (e.g., the rotary valve 350 shown in FIG. 6F) for assembling a functional cartridge. When the cup bottom cover 337 is sealed to the cup body 320 and seals the cup to form a analytic module, these ports are aligned with the ports of the bottom cover 337 (e.g., 340a and 350a as shown in FIG. 6C). The sensor chip 333 is attached to the bottom of the cup body 320 through the chip PSA 632 (FIG. 6B, left panel).

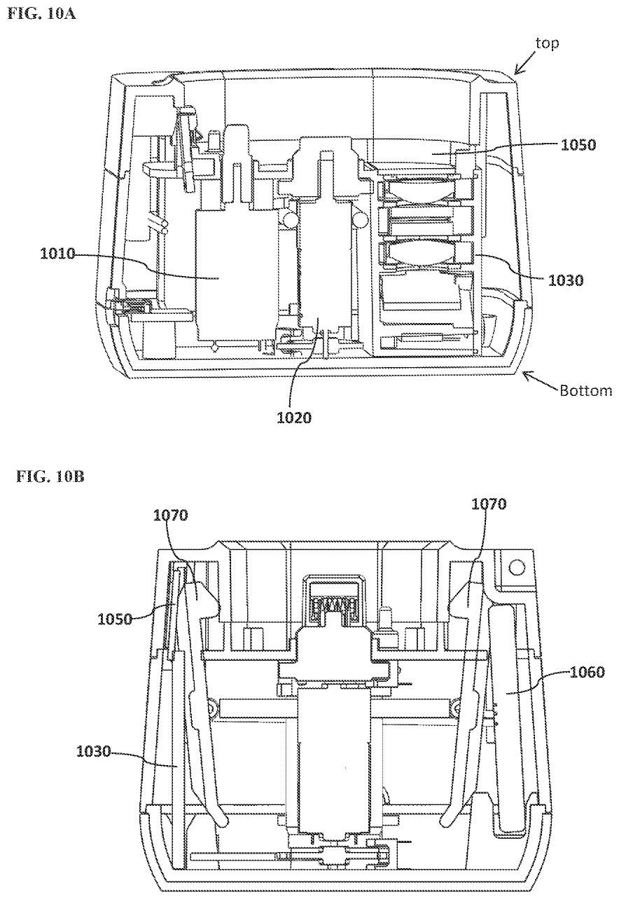

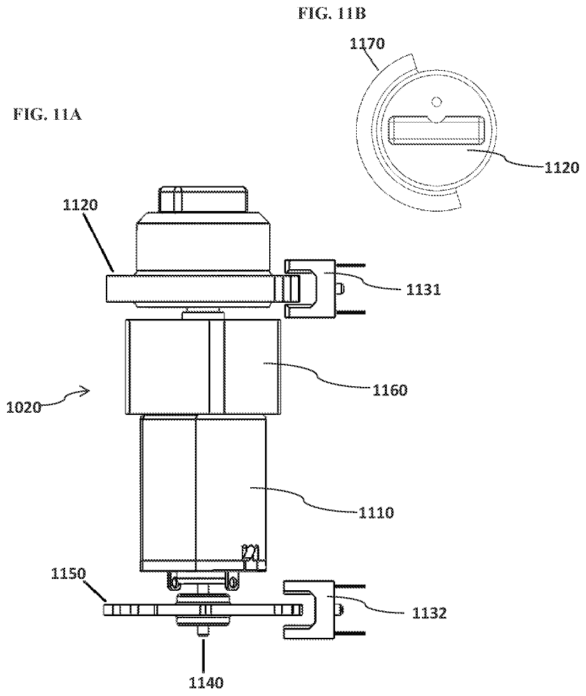

FIG. 6C shows a bottom perspective view of the cup bottom cover 337 and the bottom of the cup body 320 in alignment with each other, indicating the position of each component upon assembly of the test cup. When the bottom cover 337 and the cup body 320 are assembled together, a detection chamber with an optical window (331) is formed wherein a sensor area 332 holds the sensor chip 333. The optical window of the detection chamber 331 provides a connection to the detector unit (e.g., the detection device 100 in FIGS. 1 and 9A).

In this embodiment, the fluidic panel 631 is positioned between the bottom of the cup body 320 and the bottom cover 337 (FIG. 6A); the fluidic panel 631 may be operatively connected to a detection sensor. As a non-limiting example, the fluidic panel 631 is connected to the sensor chip 333 through the chip PSA 632 and provides essential fluid paths (e.g., 370) for flowing the processed sample to the detection chamber 331, thereby to the sensor chip 333.

In some examples, a filter assembly 325 is inserted to the homogenization chamber 321 to filtrate the processed sample. In one example, the filter assembly 325 may be the filter illustrated in FIG. 4A. In another example, the filter assembly 325 may be configured to comprise a filter 624 (e.g., a mesh filter) that is inserted to a filter gasket 623, a bulk filter 622 and a filter cap 621 (FIG. 6D). The filter assembly 325 may be fastened and controlled by the rotary valve 350 (FIG. 6E). In this embodiment, the filter cap 621 is engaged in an interaction with the threaded top of the rotary valve shaft 351 (FIG. 6E). The rotary valve 350 comprises a valve shaft 351 that is operatively connected to and locks the filter cap 621, a valve disc 352 connected to the valve shaft 351 (e.g., in FIG. 6F). The valve disc 352 is connected to a motor of the detector unit upon assembling the test cup to the detector unit.

FIG. 6G shows a bottom perspective view (upper panel) and a top perspective view (lower panel) of the cup bottom cover 337. The exterior of the bottom cover 337 holds ports (e.g., 340a and 350a) and the optical window of the sensor area 332 for connecting to the detection device 100. The interior of the bottom cover 337 includes the disc spring 613 to secure the rotary valve 350.

In some embodiments, the reaction chamber 331 at the cup bottom cover 337 may comprise a specialized sensor area 332 which is configured for holding a detection sensor for signal detection. In some aspects of the disclosure, the detection sensor may be a solid substrate (e.g., a glass surface, a chip, and a microwell) of which the surface is coated with detection probes such as short nucleic acid sequences complementary to the SPNs that bind to the target allergen. In some examples, the detection sensor held at the sensing area 332 within the reaction chamber 331 may be a glass chip 333 (as shown in FIGS. 3C and 6A).

In other embodiments, the reaction chamber 331 comprises at least one optical window. In one embodiment, the chamber comprises two optical windows, one primary optical window and one secondary optical window. Similar to the other embodiments, the primary optical window serves as the interface of the reaction chamber 331 to the detection device 100, in particular to the optical system 1030 (as shown in FIGS. 10A, 10B, and 12A-12C) of the detection device 100. The detection sensor (e.g., the glass chip 333, and the detection area 333′ of the chipannel 710) may be positioned between the optical window and the interface of the optical system. The optional secondary optical window may locate at one side of the reaction chamber 331; the secondary optical window allows detection of the background signals. In some aspects of the present disclosure, the secondary optical window may be constructed for measuring scattered light.

In some embodiments, the glass chip 333 and/or the detection area 333′ of a chipannel 710 that is printed with nucleic acid molecules (i.e., a DNA chip) is aligned with the optical window. In some embodiments, the DNA chip comprises at least one reaction panel and at least one control panel. In some aspects, the reaction panel of the chip faces the reaction chamber 331, which is flanked by an inlet and outlet channel 336 of the cartridge 300 (e.g., shown in FIGS. 3H and 3I). In some embodiments, the reaction panel of the glass chip 333 may be coated/printed with detection probes such as short nucleic acid probes that hybridize to a SPN having high specificity and binding affinity to an allergen of interest. The SPN then can be anchored to the chip upon hybridization with the nucleic acid probes.

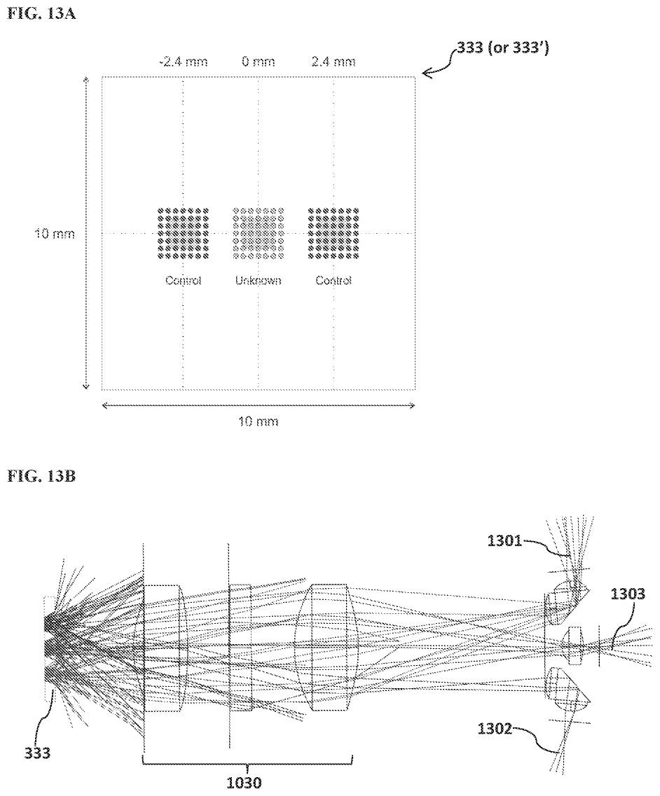

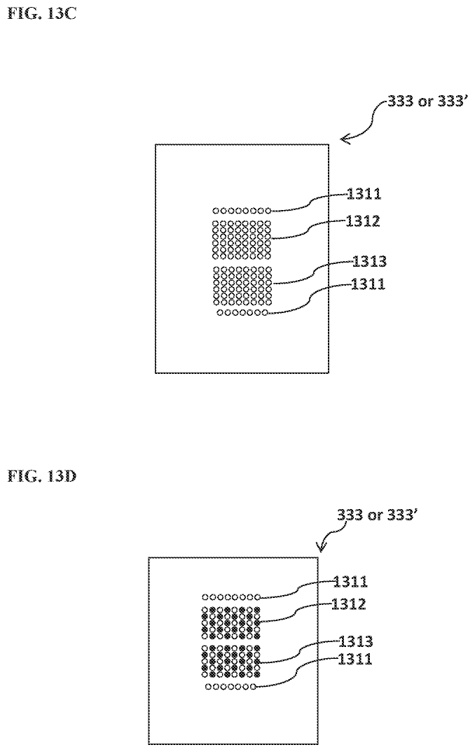

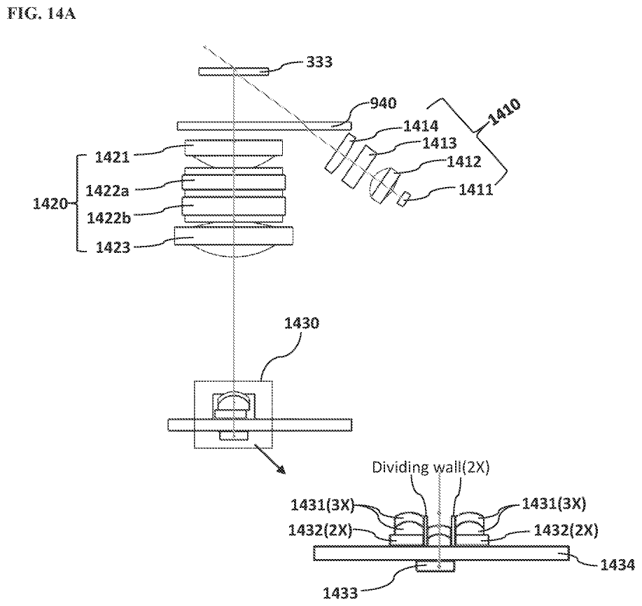

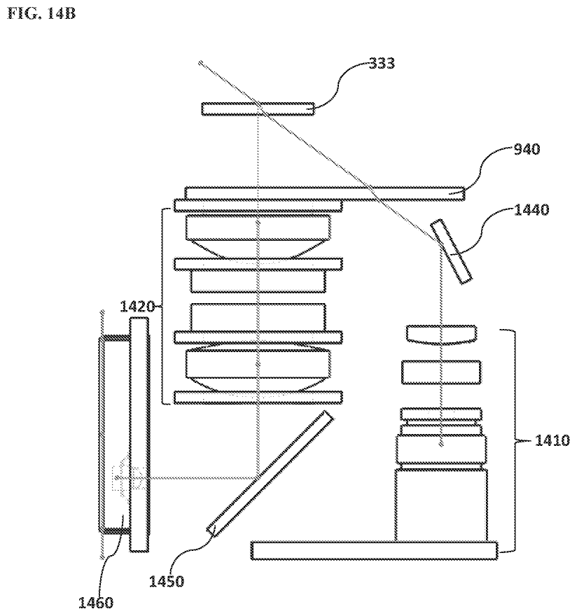

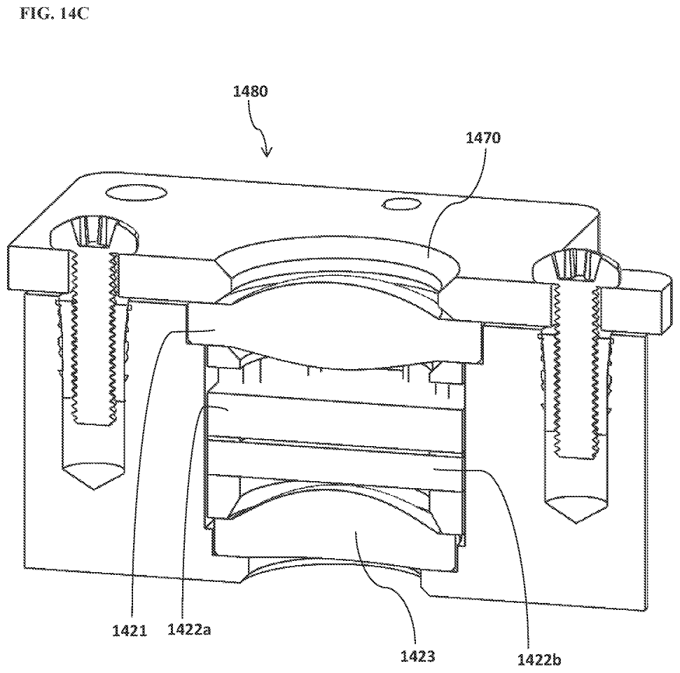

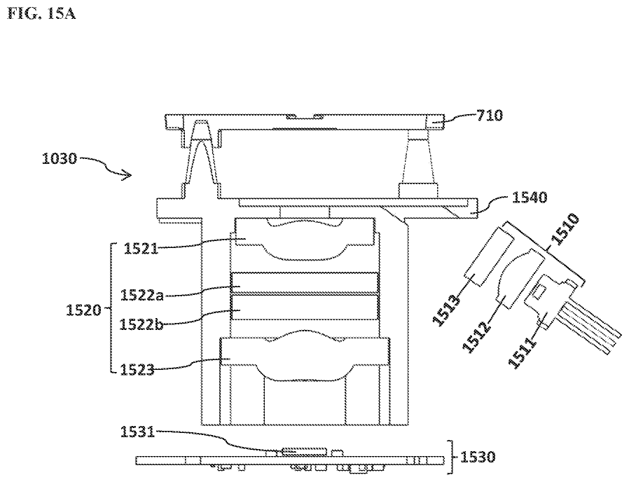

In one preferred embodiment, the sensor DNA chip (e.g., 333 in FIG. 3C, FIG. 5B and FIG. 6A, and 333′ in FIG. 7B) may comprise a reaction panel printed with detection probes comprising short complementary sequences that hybridize to a SPN specific to an allergen of interest, and two or more control areas (control panels) that are covalently-linked to nucleic acid molecules (as control probes) that do not react with the SPN or the allergen. The complementary probe sequences can only bind to the SPN when the SPN is free from binding of the target allergen proteins. In some aspects, the nucleic acid molecules printed in the control panels are labeled with a probe, for example, a fluorophore. The control panels provide an optical set-up with a mechanism to normalize signal output with respect to the reaction panel and to confirm functioning operational procedures. An exemplary configuration of the chip 333 or the detection area 333′ is illustrated in FIG. 13A.

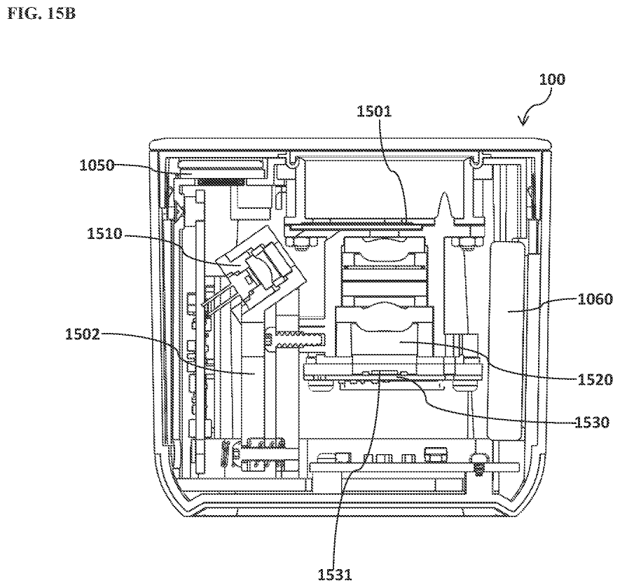

In another embodiment, the sensor DNA chip (e.g., 333 in FIG. 3C, FIG. 5B and FIG. 6A, and 333′ in FIG. 7B) may comprise one reaction panel printed with detection probes comprising short complementary sequences that hybridize to a SPN specific to an allergen of interest, one control area (control panel) that is covalently-linked to control nucleic acid molecules and one or more fiducial spots that can guide image processing and provide a self-correction mechanism for an image detector (e.g., a camera detector in FIG. 15A). An exemplary configuration of the chip 333 or the detection area 333′ is illustrated in FIG. 13C.

In some embodiments, the DNA coated chip may be pre-packed into the reaction chamber 331 of the cartridge, e.g., at the sensing area 332. In other embodiments, the DNA coated chip may be packed separately with the disposable cartridge (e.g., the cup 300 in FIG. 1). In other embodiments, the DNA chip 333 may be attached to the fluidic panel 631 shown in FIG. 6A. In other embodiments, the DNA chip may be integrated to the chipannel as a specialized detection area of the chipannel (e.g., 333′ of the chipannel 710 shown in FIG. 7B).

Another alternative embodiment of the analytical cartridge is provided in the present disclosure. The configuration of the test cup of this alternative embodiment is shown in FIG. 7A, in which the test cup 300 comprises a similar configuration of the compartments (e.g., shown in FIG. 6A) including a cup top 310, a cup body 320 that is configured to include a homogenization chamber, a filtrate chamber, wash chambers and a waste chamber, and a cup bottom 330. This design is simple and requires fewer components. In this embodiment, a chipannel 710 that combines the fluidic panel 631, the chip 333 and the chip PSA 632 into a single thin piece is provided to replace these components. The chipannel 710 may be connected to the cup body 320 through a gasket 701 (FIG. 7A) and the bottom cover 337 via a port connection 711 (FIG. 7C). Alternatively, the chipannel 710 may be welded to the cup body by a seal face 712 (e.g., in the alternative embodiment shown in FIG. 7D).

In some embodiments, the chipannel 710 comprises the fluidic paths and the sensor chip with detection probes immobilized thereon, which is made of a separate thin plastic polymer. According to the present disclosure, the chipannel 710 may be a piece of plastics in which a specific area (FIG. 7B) is configurated as the detection area 333′ (i.e., an equivalent of the separate DNA chip 333 in other embodiments). The chipannel 710 may comprise the fluidic channels (e.g., the paths 370 in FIG. 7B) connected to the detection area 333′. The detection area 333′ may be flanked by an inlet and outlet channel 336′ (FIG. 7B). The chipannel 710 may be made of optically clear resin such as COC, COP and PMMA.

In some embodiments, the nucleic acid-based detection probes are printed on the detection area 333′ of the chipannel 710 by UV irradiation. In some examples, the detection area 333′ further comprises control probes immobilized thereon. The detection probes and control probes are immobilized to form separate reaction panels and control panels. In some embodiments, the nucleic acid probes and control probes are printed on the detection area 333′ of the chipannel 710 as shown in FIG. 13C. The detection probes and control probes are printed to the reaction panel 1312 and the control panel 1313, respectively. Within each panel, the detection probes and control probes are printed in a checkerboard pattern, such as the pattern shown in FIG. 13D.

FIGS. 7C and 7D illustrate perspective views of the chipannel 710. In one embodiment, the chipannel 710 is held by a port connection 711 (FIG. 7C). A vacuum, for example, the vacuum of the detection device 100 is connected to the chipannel 710 through the port connection 711. In another embodiment, the chipannel 710 is sealed to the cup bottom 337 via a face seal 712 (FIG. 7D). The overmolding of the chipannel 710 and the cup bottom 330 will result in a seamless combination of the parts. Any overmolding and casting techniques, e.g., an injection molding process, may be used to overmold the parts into a single part.

In some embodiments, the solid substrate with detection probes immobilized thereon (e.g., chipannel 710) may be a glass with a high optical clarity such as borosilicate glass and soda glass.

In other embodiments, the solid substrate with detection probes immobilized thereon (e.g., chipannel 710) may be made of plastic materials high optical clarity. As non-limiting example, the substrate may be selected from the group consisting of polydimethylsiloxane (PDMS), cyclo-olefin copolymer (COC), polymethylmetharcylate (PMMA), polycarbonate (PC), cyclo-olefin polymer (COP), polyamide (PA), polyethylene (PE), polypropylene (PP), polyphenylene ether (PPE), polystyrene (PS), polyoxymethylene (POM), polyetheretherketone (PEEK), polytetrafluoroethylene (PTFE), polyvinylchloride (PVC), polyvinylidene fluoride (PVDF), polyvinylalcohol, polyacylate, polybutyleneterephthalate (PBT), fluorinated ethylenepropylene (FEP), perfluoroalkoxyalkane (PFA), polypropylene carbonate (PPC), polyether sulfone (PES), polyethylene terephthalate (PET), cellulose, poly(4-vinylbenzyl chloride) (PVBC), Toyopearl®, hydrogels, polyimide (PI), 1,2-polybutadiene (PB), fluoropolymers-and copolymers (e.g. poly(tetrafluoroethylene) (PTFE), perfluoroethylene propylene copolymer (FEP), Ethylene tetrafluoroethylene (ETFE)), polymers containing norbornene moieties, polymethylmethacrylate, acrylic polymers or copolymers, polystyrene, substituted polystyrene, polyimide, silicone elastomers, fluoropolymers, polyolefins, epoxies, polyurethanes, polyesters, polyethylene terephtalate, polypersulfone, and polyether ketones, and a combination thereof. The chips and chipannel may be prepared with injection mold.

In another embodiment of the test cup 300 shown FIG. 7E, the cup is further optimized to improve its performance and for manufacture. In this embodiment, the filter gasket 623 is overmolded to the interior of the cup body, e.g., in the homogenization chamber 321 (FIG. 7F). FIG. 7I demonstrates a cross-sectional view of the overmolded seal 713 that combines the parts into one single part. The overmolding facilitates the manufacturing process to result in a single piece. In this embodiment, the top of the valve shaft 351 of the rotary valve 350 comprises a cam 353 (FIG. 7G) that interacts with the filter cap 621 to provide a rotating motion (FIG. 7F, right panel). In an alternative embodiment, the top of the valve shaft of the rotary valve may be a solid stab (e.g., 351′ in FIG. 7H). FIG. 7J demonstrates the cup bottom 337 (the top panel) and the bottom perspective view of the cup body 320 (the bottom panel). In this embodiment, the rotary valve 350 is secured in the test cup body 320 through a plurality of compression coil springs 721 located at the cup bottom cover 337 (FIG. 7K). FIG. 7K further demonstrates the compression coil springs 721 at the cup bottom 337. Four coil springs 721 may locate at the corners of the rotary valve port 350a to secure the valve 350. In this embodiment, the chipannel 710 may be welded to the bottom of the cup body 320. For example, the chipannel 710 may be laser welded to the bottom of the cup body 320. FIG. 7L demonstrates, in one example, the weld bead materials 722 at the bottom of the cup body 320 for laser welding.

The cup bottom 330 is configured to close the disposable test cup 300 and to provide a means for coupling the test cup 300 to the detection device 100 in various embodiments discussed herein. In some embodiments, the bottom side of the bottom assembly 330 of the cup 300 shown in FIG. 3H, includes several interfaces for connecting the cup 300 to the detection device 100 for operation, including a homogenization rotor interface 340a that may couple the homogenization rotor 340 to a motor in the device 100 for controlling homogenization; the valve interface 350a that may couple the rotary valve 350 to a motor in the device 100 for controlling valve rotation; and a pump interface 380 for connecting to a pump in the detection device 100.

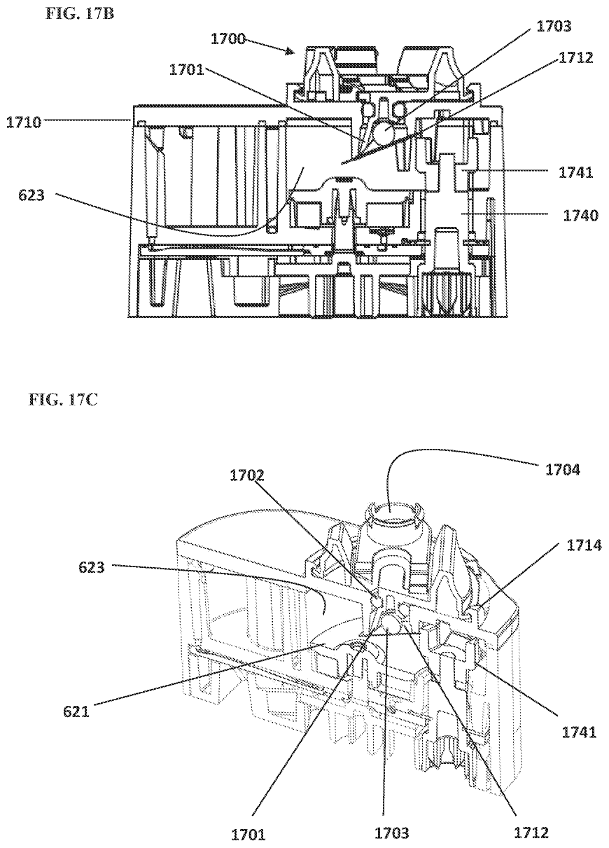

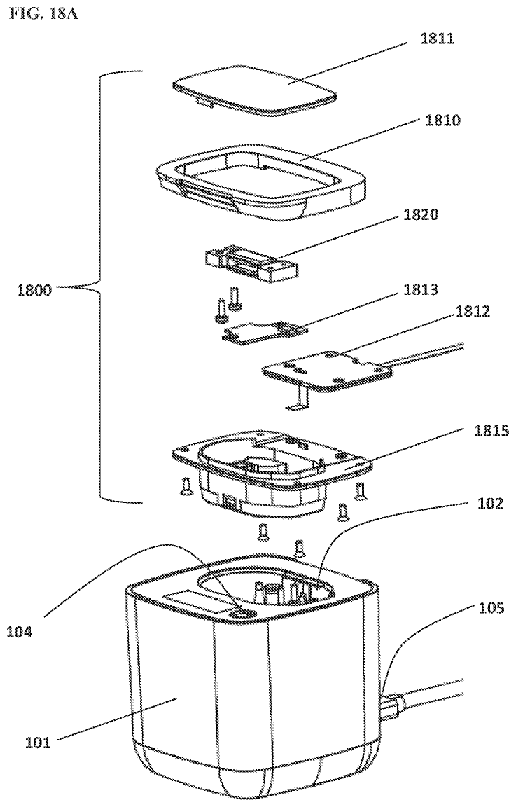

Another alternative embodiment of the analytical cartridge is provided in the present disclosure. The configuration of the test cup of this alternative embodiment is shown in FIG. 17A-17C, in which the test cup 1730 comprises a similar configuration of the compartments and other elements (e.g., shown in FIG. 7E) including a cup top or lid 1710, a cup body 320 that is configured to include a homogenization chamber 623, a filtrate chamber, wash chambers, a waste chamber, and a cup bottom 337.

In this embodiment, the cartridge includes a bead 1703 to assist or accelerate homogenization and detection. The bead may be made of any hard and non-reactive substance like metal, ceramic, or plastic. The bead may be smooth, textured, round, oblong, or any other shape to assist in homogenization. The bead may include or be coated with enzymes or other substances that assist in breaking down a sample. The embodiment includes a cap 1700 which may be removably attachable to the lid 1710 of the cartridge. The cap 1700 includes at least one bead 1703 sealed within a pocket, defined between the cap 1700 and at least one seal, which may be foil or other film 1712. The seal 1712 is bonded to a lower portion of the lid 1710. The cap 1710 further includes a piercing member, such as a blade 1701. A cross section of the embodiment is shown in FIG. 17B demonstrating the juxtaposition of the blade 1701 to the foil and the homogenization chamber. The cap 1700 is securable in and movable within a groove 1714 or another track on the top of the lid 1710, with a compliant member 1711 separating the cap 1700 and the lid 1710. The lid 1710 has an opening 1715 through which the pocket and blade 1701 extend through. The cap 1700 may be rotated within the track 1714, between 1 degree to 360 degrees, about a central opening in the lid 1710, so that the blade 1701 pierces or slices through the seal, such as foil 1712. As the foil 1712 is opened, the bead is allowed to drop into the homogenization chamber. The cap 1700 also has an O-ring 1702, which creates a secure seal between the cap 1700 and the opening 1715 in the lid 1710. This secure seal allows the reaction chamber to be isolated from the ambient environment, even after the cap 1700 has been rotated or moved, cutting the seal and releasing the bead attached to the lid 1710.

The lid 1710 may include a food port 1716 through which a sampler 200 can deposit a sample. The food port 1716 may be covered and sealed by a port seal 1713. The port seal 1713 may be broken by the food corer 200, when depositing a food sample. The cap 1700 may have a port 1704 which aligns with the food port 1716 allowing the sampler 200 to pass through the cap 1700 and the lid 1710 and into the homogenization chamber 623. In operation, once the food has been deposited into the chamber 623, the cap 1700 is rotated in the groove 1714, which causes the blade 1701 to lance the foil 1712 and drop the bead 1703 into the chamber 623. The rotation of the cap 1700 to release the bead 1703 also causes the compliant member 1711 to rotate within the groove 1714 to cover and seal the food port 1716. The O-ring 1703 and the compliant member 1711 to securely seal the homogenization chamber 623.

The embodiment in FIG. 17A also includes a two-piece homogenization rotor including the rotor base 1740 and rotor blades 1741. Base 1740 and blades 1741 may be cold welded or compression-fit together. The rotor base 1740 engages with a drivetrain to spin the rotor blades 1741. The rotor base 1740 passes through the chipannel 710 and the rotor blades 1741 are attached to the top of the rotor base 1740; the rotor blades 1741 extend into the homogenization chamber 623. The rotor blades 1741 are configured to provide more power to the food sample to break up harder substances.

The embodiment in FIGS. 17A and 17B may also include a spacer 1722 instead of a gross filter 622, as included in the embodiment of 7E.

In some embodiments, a valve system is provided to control the fluid flow of the sample, detection agents, buffers and other reagents, and waste through different parts of the cartridge (e.g., separate chambers within the cup). In addition to flexible membranes, foil seals and pinch valves discussed herein, other valves may be included to control the flow of the fluid during the process of a detection assay, including swing check valves, gate valves, ball valves, globe valves, rotary valves, custom valves, or other commercially available valves. For example, a gland seal or rotary valve 350 may be used to control the flow of the processed sample solution within the cup 300. In some examples, pinch valves or rotary valves are used to completely isolate the fluid from other internal valve parts. In other examples, air operated valves (e.g., air operated pinch valves) may be used to control the fluid flow, which are operated by a pressurized air supply.

In one embodiment, means for controlling the fluid flow within the cup chambers may be included in, for example, the cup bottom assembly 330 and/or the cup body 320. The means may comprise flow channels, tunnels, valves, gaskets, vents, and air connections. In other embodiments, the means for the fluid flow may be configured as a separate component in the cup, e.g., the fluidic panel 631 shown in FIG. 6A.

In other embodiments, the valve system of the present disclosure may comprise additional air vents included in the test cup 300, to control air flow when the DNA coated glass chip is used as the detection sensor. The DNA chip may be purged by air during the procession of an allergen detection assay. Individual air intakes may be opened based on the requirement of the system. The valve system as discussed herein may be used to keep the air vent unit inactive until use. The air port(s) allow air into the cartridge (e.g., the cup 300) and the air vent(s) allow air to enter various chambers when fluids are added to the chambers or removed from the chambers. The air vents may also have a membrane incorporated in them to prevent spillage and to act as a mechanism to control fluid fill volumes by occlusion of the vent membrane thus stopping further flow and fill function.

In one preferred embodiment, the rotary valve 350 (shown in FIG. 3C, FIG. 5B, FIGS. 6A and 6F, FIGS. 7A, 7E, and FIG. 17A) may be used to control and regulate fluid flow and rate in the test cup 300. The rotary valve 350 comprising a valve shaft 351 and a valve disc 352 (FIG. 6F and FIG. 7G) can be operated by an associated detection device (e.g., the device 100). In some embodiments, an alternative embodiment of the rotary valve 350′ as shown in FIG. 7H may be used; the valve 350′ comprises a valve shaft 351′ and valve disc 352′.

In some embodiments, the rotary valve 350 (or 350′) shown in FIGS. 6G, 7G and 7H is connected to the filter cap 621 and the rotor 340 (or the rotor base 1740) (as illustrated in FIGS. 6E and 7F). The rotation of the valve 350 control the volume and flow of the fluid in the cartridge. The valve 350 may facilitate to pull enough processed sample solution from the filter (i.e., filtrate) to the reaction chamber, particularly to the chipannel 710 for signal detection.

In some embodiments, the rotary valve 350 may position at a specific angle by rotating the valve components either counterclockwise (CCW) or clockwise (CW) at each step of the repeated washing and air purge cycle(s) during the process of a detection assay. The air hole can allow air in. Air is drawn through the system via vacuum pressure to perform air purge functions. The angle may range from about 2° to about 75°.

As a non-limiting example, the valve may be at about 38.5° as reference from the air hole wherein the pump 1040 is off and the reaction chamber 331 is dry (referred to as home position). After the test sample is processed and homogenized, the pump is on and the valve 350 is rotated CCW and parks at an angle of about 68.5°, allowing the processed sample to be transported to the filtration chamber 322. Next, the valve components may be rotated again at different directions to park at different angles such as about 57° to flow wash buffer to the reaction chamber 331, and about 72° to purge the DNA chip with air. After the prewash of the DNA chip, the valve components may be rotated to the home position at about 38.5°. The processed sample solution is pulled through the filter assembly 325. After filtration, the valve components may be rotated and park at an angle of about 2°, allowing the collected filtrate to flow into the reaction chamber 331, wherein the chemical reactions occur. The valve 350 will rotate and park at about 57° to flow wash buffer to the reaction chamber 331, and park at about 72° to purge the DNA chip with air. The wash and air purge steps may be repeated one or more times until the optical measuring indicates a clean background.

In other embodiments, the rotary valve 350 is operatively connected to a filter cap 621 (FIG. 6E), the filter cap locks the rotary valve 350, for example during the shipment of the test cup 300.

In one embodiment, the valve system may be a rotary valve as shown in FIG. 8A and FIG. 8B. In this embodiment, the rotary valve 350 is positioned to control air in and fluid flow. The positioning may drive the homogenization in the homogenization chamber 321, filtration and collection of filtrates (F), sample washes (e.g., wash 1 (W1) and wash 2 (W2) and waste collection (in FIG. 8A). In step 1 of FIG. 8B, the rotary valve 350 is in a closed position with no connections being made between any of the chambers. In step 2 of FIG. 8B, the rotary valve 350 connects the wash 1 chamber W1 to the reaction chamber 331 to flush the reaction chamber 331 with the wash buffer subsequently being pushed out to the waste chamber 323. In step 3 of FIG. 8B, the rotary valve 350 connects the homogenization chamber 321 to the filtrate chamber F to affect the filtration step. In step 4 of FIG. 8B, the rotary valve 350 connects the filtrate chamber F to the reaction chamber 331 to send the filtrate to the reaction chamber 331 for reaction and analysis. In step 5 of FIG. 8B, the rotary valve 350 connects the wash 2 chamber W2 to the reaction chamber to flush the reaction chamber 331 again.

In some embodiments, extraction buffers may be pre-stored in the analytic cartridge, e.g., the homogenization chamber 321 of the cup body 320, for example in foil sealed reservoirs like the food processing reservoir 801 (FIG. 8C). Alternatively, extraction buffers may be stored separately in a separate buffer reservoir in the cup body 320, a reservoir similar to the wash buffer storage reservoir 802 (in the buffer storage chamber 324 (optional) as shown in FIG. 8C). The extraction buffer after sample homogenization and washing waste may be stored in the separate waste reservoir 803 within the waste chamber 323. The waste chamber 323 has sufficient volume to store a volume greater than the amount of fluid used during the detection assay.

In accordance with the present disclosure, the homogenization rotor 340 may be constructed to be small enough to fit into a disposable test cup 300, particularly into the homogenization chamber 321, where the homogenizer processes a sample to be tested. Additionally, the homogenization rotor 340 may be optimized to increase the efficacy of sample homogenization and protein extraction. In one embodiment, the homogenization rotor 340 may comprise one or more blades or the equivalent thereof at the proximal end. In some examples, the rotor 340 may comprise one, two, three or more blades. The homogenization rotor 340 is configured to pull the test sample from the food corer 200 into the bottom of the homogenization chamber 321.

Alternatively, the homogenization rotor 340 may further comprise a center rod running through the rotor that connects through the cup body 320 to a second interface bit. The central rod may act as an additional bearing surface or be used to deliver rotary motion to the rotor 340. When the rotor 340 is mounted to the cup body through the port at the cup bottom (e.g., 340a), the blade tips may remain submersed within the extraction buffer during operation. In another alternative embodiment, the homogenization rotor 340 may have an extension to provide a pass through the bottom of the cup; the pass may be used as a second bearing support and/or an additional location for power transmission. In this embodiment, the lower part of the rotor has a taper to fit to a shaft, forming a one-piece rotor. In accordance with the present disclosure, depth of the blades of the homogenization rotor 340, with or without the center rod, is constructed to ensure the blade tips in the fluid during sample processing.

As compared to other homogenizers (e.g., U.S. Pat. No. 6,398,402, incorporated herein by reference in its entirety), the custom blade core of the present disclosure spins and draws and forces food into the toothed surfaces of the custom cap. The homogenizer rotor may be made of any thermoplastics, including, but not limited to, polyamide (PA), acrylanitrilebutadienestyrene (ABS), polycarbonate (PC), high Impact polystyrene (HIPS), and acetal (POM).

The disposable cartridge may be in any shape, for example, circular, oval, rectangular, or egg-shaped. Any of these shapes may be provided with a finger cut or notch. The disposable cartridge may be asymmetrical, or symmetrical.

Optionally, a label or a foil seal may be included on the top of the cup lid 311 to provide a final fluid seal and identification of the test cup 300. For example, a designation of peanut indicates that the disposable test cup 300 is used for detecting the peanut allergen in a food sample.

### The Detection Device

In some embodiments, the detection device 100 may be configured to have an external housing 101 that provides support surfaces for the components of the detection device 100; and a lid 103 that opens the detection device 100 for inserting a disposable test cup 300 and covers the cup during operation. The small lid may be located at one side of the device (as shown in FIG. 1 and FIG. 9A), or in the center (not shown). In some aspects of the disclosure, the lid may be transparent, allowing all the operations visible through the lid 103. The device may also comprise s USB port 105 for transferring data.

One embodiment of the allergen detection device 100 according to the present disclosure is depicted in FIG. 1 and FIG. 9A. As illustrated in FIG. 1, the detection device 100 comprising an external housing 101 that provides support for holding the components of the detection device 100 together. The external housing 101 may be formed of plastic or other suitable support material. In other embodiments, the device may be made of Aluminum. The device also has a port or receptacle 102 for docking the test cup 300 (FIG. 1 and FIG. 9A).

To execute an allergen detection test, the detection device 100 is provided with a means (e.g., a motor) for operating the homogenization assembly and necessary connectors that connect the motor to the homogenization assembly; means (e.g., a motor) for controlling the rotary valve; means for driving and controlling the flow of the processed sample solution during the process of the allergen detection test; an optical system; means for detecting fluorescence signals from the detection reaction between the allergen in the test sample and the detection agents; means for visualizing the detection signals including converting and digitizing the detected signals; a user interface that displays the test results; and a power supply.

As viewed from the transparent lid 103 (FIG. 9A), the device 100 has an interface comprising areas for coupling the components of the cartridge 300 (when inserted) for operating a detection reaction (FIG. 9B). These areas include a homogenization bit 910 for coupling the rotor 340 to the motor, a vacuum bit 920 for coupling the cup with the vacuum pump, a rotary valve drive bit 930 for coupling the rotary valve 350 to a valve motor and a protective glass 940 which is aligned to the glass chip 333 or the sensor area 333′ of the chipannel 710 through the optical window of the reaction chamber 331. A data chip reader 950 is also included to read the data chip 335. The pins 960 are used to facilitate placement of the cup 300 in the receptacle of the device 100.

In one embodiment of the present disclosure, as shown in FIG. 10A, the components of the detection device 100 that are integrated to provide all motion and actuation for operating a detection reaction, include a motor 1010 which may be connected to the homogenization rotor 340 inside the homogenization chamber 321 within the cup body 320. The motor 1010 may be connected through a multiple-component coupling assembly including a gear train/drive platen for driving the rotor during homogenization in an allergen detection test; a valve motor 1020 for driving the rotary valve 350; an optical system 1030 that is connected to the reaction chamber 331 (not shown) or the chipannel 710 within the disposable test cup 300; a vacuum pump 1040 for controlling and regulating air and fluid flow (not shown in FIG. 10A), a PCB display 1050, and a power supply 1060 (in FIG. 10B). A means for retaining the test cup (i.e. the cup retention 1070) is included for holding the test cup 300. Each part is described below in detail.

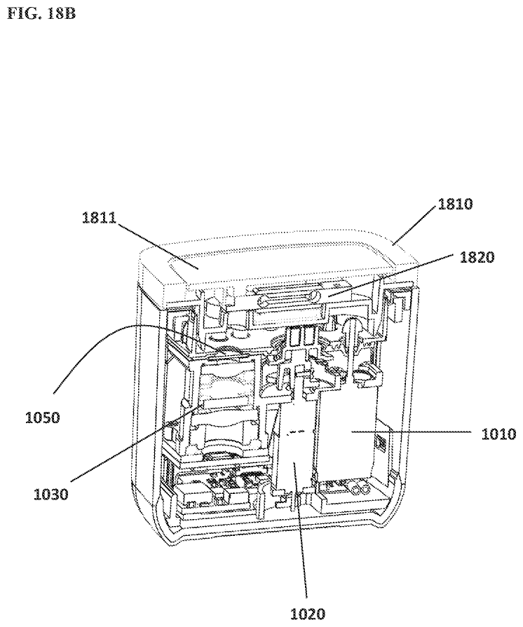

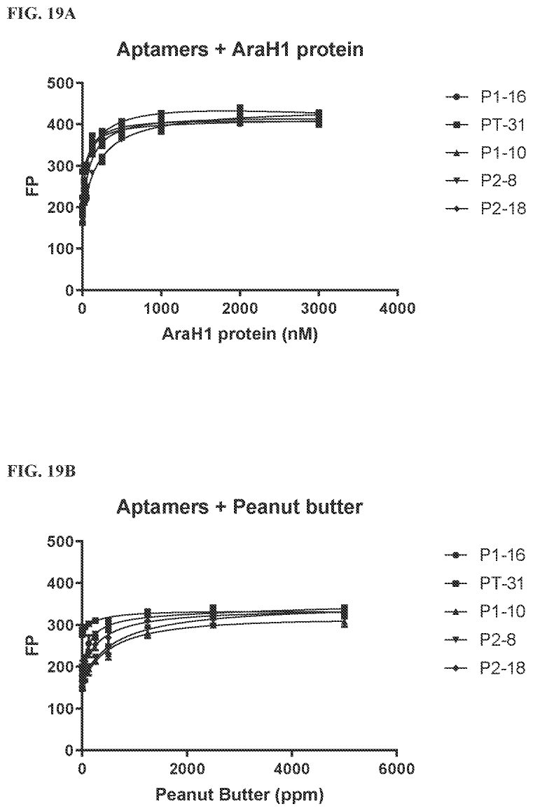

In one embodiment of the present disclosure, as shown in FIGS. 18A and 18B, the detection device 100 may include an integrated scale assembly 1800 as a lid for the device 100. The scale assembly may be operable to determine the weight, mass, or volume of the food sample to be analyzed in the device, prior to engaging the full assay of the device. By determining the weight of the food, a user can determine if the proper amount of food has been captured before turning on the assay. This prevents the waste of an assay, reagents, or a pod.

The integrated scale assembly 1800 may include a frame 1810 for connecting all the elements of the assembly 1800. A cover 1811 acts as the outer most lid of the device 100 as well as being operable as the scale surface onto which the food sample will be placed. The cover 1811 is supported by the frame 1810. The bottom of the cover 1811 rests on a strain gauge 1820 or other weight or mass sensing device. The strain gauge 1820 determines the weight of the food by parallel elements deflecting from a first neutral position. The extent of deflection of the elements allow for a measurable factor to be converted into weight. The processing may occur in electronics integrated within the integrated scale. The electronics within lid 1800 or within the housing 101 converts the analog output from the strain gauge 1820 to a digital output, rendering the weight. The strain gauge 1820 is supported by a gasket 1813 and a platform 1812, which may be operable to provide feedback to the strain gauge 1820 to assist in determining weight of the sample. The lid 1800 may draw power through a connection with the powered base 101 or the lid may have independent power and connectivity through the platform 1812. The lid 1800 and components therein are supported by the base 1815. It is within the conception of this embodiment that the lid 1800 includes other forms of scales or weight sensing devices, such as springs, load cells, laser vibrometry, accelerometer, driven coil, or other appropriately sized and configured weight measurement device. It is within the scope of this embodiment that multiple measurement device may be combined along with software to calculate or determine other characteristics of the sample, such as mass, volume, pH, density, hardness, moisture content, texture, or other factors useful for analysis and detection.

In operation, a user would place a portion of the food sample on the cover 1811 of the lid 1800. The food exerts a force onto the cover 1811 and the strain gauge 1820 measures the displacement or the weight of the food sample. The lid with integrated scale 1800 may be integrated with the device 100 to give feedback to the system prior to beginning a detection assay. In order to prevent a wasted assay, software running the device would lock out the initiation of an assay until a sample of sufficient weight is measured. The lid 1800 is adapted to fit in the device 100, as shown in FIG. 10A, and integrate amongst the other components as shown in FIG. 18B. The frame 1810 including the strain gauge 1820 may fit within the receptacle 102. The optical system 1030 and display 1050 fit under the lid 1800. The homogenization motor 1010 and the valve motor 1020 have ample space in the device below the integrated lid 1800.

### 1. Homogenization Assembly

In one embodiment, the motor 1010 may be connected to the homogenization rotor 340 inside the test cup 300 through the multiple-component rotor coupling assembly. The rotor coupling assembly may include a coupling that is directly linked to the distal end cap of the rotor 340, and a gearhead that is part of a gear train or a drive (not shown) for connection to the motor 1010. In some embodiments, the coupling may have different sizes at each end, or the same sizes at each end of the coupling. The distal end of the coupling assembly may connect to the rotor 340 through the rotor port 340a at the cup bottom 330. It is also within the scope of the present disclosure that other alternative means for connecting the motor to the homogenization rotor 340 may be used to form a functional homogenization assembly.

In some embodiments, the motor 1010 can be a commercially available motor, for example, Maxon motor systems: Maxon RE-max and/or Maxon A-max (Maxon Motor ag, San Mateo, CA, USA).

Optionally, a heating system (e.g. resistance heating, or peltier heaters) may be provided to increase the temperature of homogenization, therefore, to increase the effectiveness of sample dissociation and shorten the processing time. The temperature may be increased to between 60° C. to 95° C., but below 95° C. Increased temperature may also facilitate the binding between detection molecules and the allergen being detected. Optionally a fan or peltier cooler may be provided to bring the temperature down quickly after implementing the test.

The motor 1010 drives the homogenization assembly to homogenize the test sample in the extraction buffer and dissociate/extract allergen proteins. The processed sample solution may be pumped or pressed through the flow tubes to next chamber for analysis, for example, to the reaction chamber 331 in which the processed sample solution is mixed with the pre-loaded detection molecules (e.g., aptamer-magnetic bead conjugates) for the detection test. Alternatively, the processed sample solution may first be pumped or pressed through the flow tubes to the filter assembly 325 and then to the filtrate chamber 322 before transported to the reaction chamber 331 for analysis.

### 2. Filtration

In some embodiments, means for controlling the filtration of the processed test sample may be included in the detection device. The food sample will be pressed through a filter membrane or a filtering assembly before the extraction solution being delivered to the reaction chamber 331, and/or other chambers for further processing such as washing. One example is the filter membrane(s). The membranes provide filtration of specific particles from the processed protein solution. For example, the filter membrane may filter particles up to from about 0.1 μm to about 1000 μm, or about 1 μm to about 600 μm, or about 1 μm to about 100 μm, or about 1 μm to about 20 μm. In some examples, the filter membrane may remove particles up to about 20 μm, or about 19 μm, or about 18 μm, or about 17 μm, or about 16 μm, or about 15 μm, or about 14 μm, or about 13 μm, or about 12 μm, or about 11 μm, or about 10 μm, or about 9 μm, or about 8 μm, or about 7 μm, or about 6 μm, or about 5 μm, or about 4 μm, or about 3 μm, or about 2 μm, or about 1 μm, or about 0.5 μm, or about 0.1 μm. In one example, the filter membrane may remove particles up to about 1 μm from the processes sample. In some aspects, filter membranes may be used in combination to filter specific particles from the assay for analysis. This filter membrane may include multistage filters. Filter membranes and/or filter assemblies may be in any configuration relative to the flow valve. For example, the flow valves may be above, below or in between any of the stages of the filtration.

In some embodiments, the filter assembly may be a complex filter assembly 325 as illustrated in FIG. 4A in which the processed sample is filtered sequentially through the gross filter 411, the depth filter 412 and the membrane filter 420. In other embodiments, the filter assembly 325 may the filter stack shown in FIG. 6D.

### 3. Pump and Fluid Motion

In accordance with the present disclosure, a means for driving and controlling the flow of the processed sample solution is provided. In some embodiments, the means may be a vacuum system or an external pressure. As a non-limiting example, the means may be a platen (e.g., a welded plastic clamshell) configured to being multifunctional in that it could support the axis of the gear train and it could provide the pumping (sealed air channel) for the vacuum to be applied from the pump 1040 to the test cup 300. The pump 1040 may be connected to the test cup 300 through the pump port 920 located at the bottom (FIG. 9B), which connects to the pump interface 380 (FIG. 3G) on the bottom 330 of the test cup 300 when the cup is inserted to the device.

The pump 1040, such as piezoelectric micro pump (e.g., Takasago Electric, Inc., Nagoya, Japan), or peristaltic pump, may be used to control and automatically adjust the flow to a target flow rate. The flow rate of a pump is adjustable by changing either the driver voltage or drive frequency. As a non-limiting example, the pump 1040 may be a peristaltic pump. In another embodiment the pump 1040 may be is a piezo pump currently on the market that have specifications that indicate they could be suitable for the aliquot function required to flow filtered sample solution to different chambers inside the test cup 300. The pump 1040 may be a vacuum pump or another small pump constructed for laboratory use such as a KBF pump (KNF Neuberger, Trenton, NJ, USA).

Alternatively, a syringe pump, diaphragm and/or a micro-peristaltic pump may be used to control fluid motion during the process of a detection assay and/or supporting fluidics. In one example, an air operated diaphragm pump may be used. The pump is driven by an electronic motor such as DC brushed motor.

### 4. Rotary Valve Control

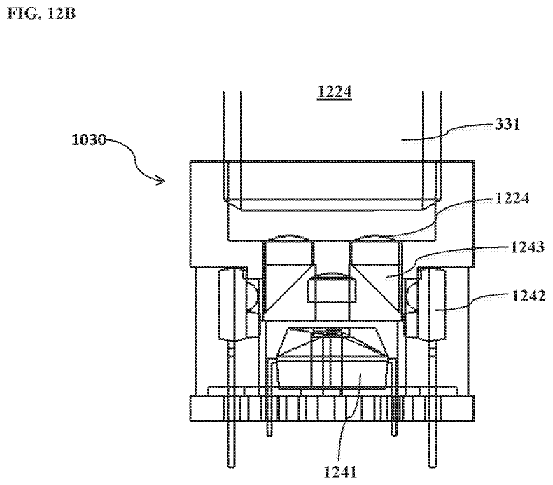

In some embodiments, the rotary valve 350 (e.g., as shown in FIG. 6F) for controlling fluid flow needs to be in precise positions. A means to control the rotary valve is provided and the control mechanism is able to rotate the valve in both directions and accurately stop at desired locations. In some embodiments, the device 100 includes a valve motor 1020 (in FIG. 10A). As shown in FIG. 11A, the valve motor 1020 may be a low cost, DC geared motor 1110 with two low cost optical sensors (1131 and 1132), and a microcontroller. An output coupling 1120 interfaces with the rotary valve 350. In some embodiments, the output coupling 1120 has a ‘half-moon’ shelf 1170 as shown in FIG. 11B, which interrupts the output optical sensor 1131 with the protruding half. The output optical sensor signal toggles between high and low, depending on whether or not the protruding shelf interrupts the sensor. A microcontroller (MCU) detects these transitions and get an absolute position of the output from this signal. The positioning of these transitions is important and application specific since these transitions are used during directional changes to account for gear backlash.

The direct motor shaft 1140 has a paddle wheel which interrupts the direct shaft optical sensor 1132, allowing the direct shaft optical sensor 1132 to output a train of pulses, with the number of pulses per revolution determined by the number of paddles on the wheel 1150. The MCU reads this train of pulses and determines the degrees movement of the output coupling. The resolution is dependent on the number of paddles of the direct shaft encoder wheel 1150, and the gear reduction ratio of the gearbox 1160.

The MCU interprets the output of these two optical sensors and can drive the output to a desired location, as long as the position of the output coupling shelf transitions, the number of paddle wheels on the direct encoder wheel 1120, and the gear ratio are known. During a change of direction, the motor must rotate by a fixed amount before an output transition is seen, the fixed amount is selected to overcome backlash of the gears. Once the fixed amount is overcome, on the next output signal transition, the MCU can start counting the direct signal pulses with confidence that they correspond to accurate output of location and movement.

### 5. Optical System

In practice, engineered molecules, i.e. aptamers, seek out proteins. The molecules have “heads” that bind to either a protein of interest or an anchor. When bound to the anchor, the molecules are able to fluoresce; the fluorescence being detectable. With no allergen present the molecules bind to the anchor resulting in higher fluorescence. When an allergen is present the molecules attach to the allergen, preventing the molecules from binding to the anchor. This results in a low or no fluorescence. The device and detection unit then presents the levels of fluorescence as positive or negative for the allergen.

The detection device 100 of the present disclosure comprises an optical system that detects optical signals (e.g., a fluorescence signal) generated from the interaction between an allergen in the sample and detection agents (e.g., aptamers and SPNs). The optical system may comprise different components and variable configurations depending on the types of the fluorescence signal to be detected. The optical system is close to and aligned with the detection cartridge, for instance, the primary optical window and optionally the secondary optical window of the reaction chamber 331 of the test cup 300 as discussed above.

In some embodiments, the optical system 1030 may include excitation optics 1210 and emission optics 1220 (FIGS. 12A and 12B). In one embodiment, as shown in FIG. 12A, the excitation optics 1210 may comprise a Light Emitted Diode (LED) 1211 configured to transmit an excitation optical signal to the sensing area (e.g., 332) in the reaction chamber 331, a collimation lens 1212 configured to focus the light from the light source, a filter 1213 (e.g., a bandpass filter), a focus lens 1214, and an optional LED power monitoring photodiode. The emission optics 1220 may comprise a focus lens 1221 configured to focus at least one portion of the allergen-dependent optical signal onto the detector (photodiode), two filters including a longpass filter 1222 and a bandpass filter 1223, a collection lens 1224 configured to collect light emitted from the reaction chamber and an aperture 1225. The emission optics collects light emitted from the solid surface (e.g. a DNA chip 333) in the detection chamber 331 and the signal is detected by the detector 1230 configured to detect an allergen-dependent optical signal emitted from the sensing area 332. In some aspects, the excitation power monitoring may be integrated into the LED (not shown in FIG. 12A).

A light source 1211 is arranged to transmit excitation light within the excitation wavelength range. Suitable light sources include, without limitation, lasers, semi-conductor lasers, light emitting diodes (LEDs), and organic LEDs.

An optical lens 1212 may be used along with the light source 1211 to provide excitation source light to the fluorophore. An optical lens 1214 may be used to limit the range of excitation light wavelengths. In some aspects, the filter may be a band-pass filter.

Fluorophore labeled SPNs specific to a target allergen are capable of emitting, in response to excitation light in at least one excitation wavelength range, an allergen-binding dependent optical signal (e.g. fluorescence) in at least one emission wavelength range.

In some embodiments, the emission optics 1220 are operable to collect emissions upon the interaction between detection agents and target allergens in the test sample from the reaction chamber 331. Optionally, a mirror may be inserted between the emission optics 1220 and the detector 1230. The mirror can rotate in a wide range of angles (e.g., from 1° to 90°) which could facilitate formation of a compacted optical unit inside the small portable detection device.

In some embodiments, more than one emission optical system 1220 may be included in the detection device. As a non-limiting example, three photodiode optical systems may be provided to measure fluorescence signals from an unknown test area and two control areas on a glass chip (e.g., see FIG. 13B). In other aspects, an additional collection lens 1224 may be further included in the emission optics 1220. This collection lens may be configured to detect several different signals from the chip 333. For example, when the detection assay is implemented using a DNA glass chip, more than two control areas may be constructed on the solid surface in addition to a detection area for allergen detection. The internal control signals from each control area may be detected at the same time when an allergen derived signal is measured. In this context, more than two collection lenses 1224 may be included in the optical system 1030, one lens 1224 for signal from the detection area and the remaining collection lenses 1224 for signals from the control areas.

The detector (e.g., photodiode) 1230 is arranged to detect light emitted from the fluidic chip in the emission wavelength range. Suitable detectors include, without limitation, photodiodes, complementary metal-oxide-semiconductor (CMOS) detectors, photomultiplier tubes (PMT), microchannel plate detectors, quantum dot photoconductors, phototransistors, photoresistors, active-pixel sensors (APSs), gaseous ionization detectors, or charge-coupled device (CCD) detectors. In some aspects, a single and/or universal detector can be used.

In some embodiments, the detector 1230 may be an image detector, such as a camera as described herein below.

In some embodiments, the optical system 1030 may be configured to detect fluorescence signals from the solid substrate sensor (e.g., DNA chip 333 shown in FIG. 13A or the chipannel 710 shown in FIGS. 7A to 7C). The DNA chip may be configured to contain a central reaction panel which is marked as an “unknown” signal area on the chip (FIG. 13A), and at least two control areas at various locations of the chip (FIG. 13A). In this context, the optical system 1030 is configured to measure both detection signals and internal control signals simultaneously (FIG. 13B).

In one example, the optical system 1030 comprises two collection lenses 1224 and corresponding optical components, such as control array photodiodes for each lens 1224. FIG. 12B demonstrates a side view of the optical system 1030 shown in FIG. 12A inside the detection device 100. In this embodiment, two collection lenses 1224 are included in the optical system, one for collecting control array signals from the DNA chip (e.g., the two signals 1301 and 1302 shown in FIG. 13B) and one specific to the unknown detection signal from the DNA chip (e.g., the detection signal 1302 as shown in FIG. 13B). In other aspects, the collection lenses 1224 may be configured to collecting signals from the detection area 333′ of the chipannel 710, e.g., one signal from the reaction panel 1312 and the other signal from the control panel 1313 shown in FIG. 13C. A signal array diode 1241 (e.g., the LED diode 1211 shown in FIG. 12A) and two control assay photodiodes 1242 are included for each optical path. Additionally, two prisms 1243 may be added to the two collection-lenses (1224) configured for collecting signals from the two control areas. The prisms 1243 can bend the control array light to the photodiode sensor area.

In some embodiments, the optical system 1030 may be configured as a straight mode as shown in FIG. 14A. The excitation optics 1410, which are configured to transmit an excitation optical signal to the glass chip 333 (e.g., DNA coated chip) in the reaction chamber 331, may comprise a LED 1411, a collimation lens 1412, a bandpass filter 1413 and a cylinder lens 1414. The cylinder lens 1414 may cause the excitation light to form a line to cover the reaction panel and control panels on the glass chip (e.g., FIG. 13B). The emission optics 1420 which are aligned with the glass chip 333 may comprise a collection lens 1421 configured to collect light emitted from the glass chip 333, a bandpass filter 1422a, a longpass filter 1422b, and a focus lens 1423 configured to focus at least one portion of the allergen-dependent optical signal onto the chip reader 1430. The chip reader 1430 is composed of three photodiode lenses 1431, two control array photodiodes 1432, a signal array photodiode 1433 and a collection PCB 1434 (FIG. 14A). In some embodiments, the collection lens 1421 may be shaped to contain a concave first surface to optimize imaging and minimize stray light.

As a non-limiting example, the excitation optics 1410 and the emission optics 1420 may be folded and configured into a stepped bore 1480 in the device 100 (see FIG. 14C). An excitation folding mirror 1440 and a collection folding mirror 1450 may be configured to minimize the light paths from the excitation optics 1410 and the emission optics 1420, respectively (in FIG. 14B). The minimized volume can modulate the laser at a frequency to minimize interference from environmental light sources. A photodiode shield 1460 may be added to cover and protect the chip reader 1430 shown in FIG. 14A. The reader 1430 is then positioned close to the collection lens 1421 to minimize the scattered light. FIG. 14C illustrates an example of the stepped bore 1480 in the device to hold the emission optics 1420. The aperture 1470 of the collection lens 1421 is shown in FIG. 14C.

The LED source (e.g., 1411) may be modulated, and/or polarized and oriented to minimize the reflections from the glass chip. Accordingly, the chip reader may be synchronized to measure modulated light.

FIG. 15A illustrates another embodiment of the optical system 1030. In this embodiment, the optical system 1030 comprises an image detector. The image detector may be a camera 1531, as part of the signal reader 1530. The camera may catch the reaction images of the sensor DNA chip 333 or the detection area 333′ of the chipannel 710. As a non-limiting example, the optical system 1030 shown in FIG. 15A, comprises an excitation optics 1510 comprising excitation filter 1513, collimation lens 1512 and laser diode 1511, an emission optics 1520 comprising a collection lens 1521, bandpass filter 1522a, longpass filter 1522b (e.g., color glass longpass filter) and focus lens 1523, and a signal reader 1530 comprising a camera 1531. Each system of the optical system may be configured in an optical housing, e.g., the optical housing 1540 in FIG. 15A configured for holding the components of the emission optics 1520.

FIG. 15B illustrates a cross-sectional view of the optical system of FIG. 15A assembled inside the detection device 100. From this cut-away side view, the excitation optics 1510 and the emission optics 1520 are assembled into an optical housing, respectively. A protective window 1501 may be added to protect the optical components. Optionally, a laser adjustment mount 1502 may be included to adjust the laser diode 1511 inside the excitation optics 1510. The camera 1531 catches the reaction images and the raw images are collected and processed. The detection results may be displayed through the display PCB 1050.

The above described optical system 1030 is illustrative examples of certain embodiments. Alternative embodiments might have different configurations and/or different components.

In other embodiments, a computer or other digital control system can be used to communicate with the light filters, the fluorescence detector, the absorption detector and the scattered detector. The computer or other digital control systems control the light filter to subsequently illuminate the sample with each of the plurality of wavelengths while measuring absorption and fluorescence of the sample based on signals received from the fluorescence and absorption detectors.

### 6. Display

As shown in a cut-away side view in FIG. 10B, a printed circuit board (PCB) 1050 is connected to the optical system 1030. The PCB 1050 may be configured to be compact with the size of the detection device 100 and at the same time, may provide enough space to display the test result.

Accordingly, the test result may be displayed with back lit icons, LEDs or an LCD screen, OLED, segmented display or on an attached cell phone application. The user may see an indicator that the sample is being processed, that the sample was processed completely (total protein indictor) and the results of the test. The user may also be able to view the status of the battery and what kind of cartridge is placed in the device (bar code on the cartridge or LED assembly). The results of the test will be displayed, for example, as (1) actual number ppm or mg; or (2) binary result yes/no; or (3) risk analysis—high/medium/low or high/low, risk of presence; or (4) range of ppm less than 1/1-10 ppm/more than 10 ppm; or (5) range of mg less than 1 mg/between 1-10 mg/more than 10 mg. The result might also be displayed as number, colors, icons and/or letters.

In accordance with the present disclosure, the detection device 100 may also include other features such as means for providing a power supply and means for providing control of the process. In some embodiments, one or more switches are provided to connect the motor, the micropump and/or the gear train or the drive to the power supply. The switches may be simple microswitches that can turn the detection device on and off by connecting and disconnecting the battery.

The power supply 1060 may be a Li-ion AA format battery or any commercially available batteries that are suitable for supporting small medical devices such as the Rhino 610 battery, the Turntigy Nanotech High dischargeable Li Po battery, or the Pentax D-L163 battery.

In the description herein, it is understood that all recited connections between components can be direct operative connections or indirectly operative connections. Other components may also include those disclosed in the applicant's U.S. Provisional application 62/461,332, filed on Feb. 21, 2017; the contents of which are incorporated herein by reference in their entirety.

The allergen detection system may create a feedback loop for all stakeholders. The stakeholders may include a user, the user's family, caregivers, health care providers, or another party to whom data access is important (such as researchers). The system allows a user to input personal data into a user interface, such as on a smartphone. The system is then able to crowdsource data, which includes sharing the data to interested parties. The crowdsourcing may also allow for feedback in a consumer app, so that other users become aware of foods or restaurants that have a source of allergens or may be considered clear of the allergen. This access may assist interested users in deciding which foods, sources of foods, or restaurants may be considered safe from the allergens.

The system may create a neural network of users' feedback and results from certain allergen tests. Each n\hat{ }th test by a user makes the testing algorithm more accurate. The neural network of data creates a competitive insulation to protect individual data if warranted, to alleviate HIPAA concerns. The crowdsourcing and neural networking of data creates a virtuous data loop beginning including 1) device software modifications, 2) food specifications from brands, 3) user food testing data, 4) leveraging of actionable information, and 5) algorithm improvements.

### Detection Assays

In another aspect of the present disclosure, provided is an allergen detection test implemented using detection assemblies and systems, detection agents and detection sensors of the present disclosure.

As a non-limiting example, an allergen detection test comprises the steps of (a) collecting a certain amount of a test sample suspected of containing an allergen of interest, (b) homogenizing the sample and extracting allergen proteins using an extraction/homogenization buffer, (c) contacting the processed sample with a detection agent that specifically binds to a target allergen; (d) contacting the mixture in (c) with a detection sensor comprising a solid substrate that is printed with nucleic acid probes; (e) measuring fluorescence signals from the reaction; and (f) processing and digitizing the detected signals and visualizing the interaction between the detection agents and the allergen.

In some aspects of the disclosure, the method further comprises the step of washing off the unbound compounds from the detection sensor to remove any non-specific binding interactions.

In some aspects of the disclosure, the method further comprises the step of filtering of the processed sample prior to contacting it with the detection sensor (e.g., DNA chip).

In some embodiments, an appropriately sized test sample is collected for the detection assay to provide a reliable and sensitive result from the assay. In some examples, a sampling mechanism that can collect a test sample effectively and non-destructively for fast and efficient extraction of allergen proteins for detection is used.

A sized portion of the test sample can be collected using, for example, a food corer 200 illustrated in FIG. 2B. The food corer 200 collect an appropriately sized sample from which can be extracted sufficient protein for the detection test. The sized portion may range in mass from 0.1 g to 1 g, preferably 0.5 g. Furthermore, the food corer 200 may pre-process the collected test sample by cutting, grinding, blending, abrading, and/or filtering. Pre-processed test sample will be introduced into the homogenization chamber 321 for processing and allergen protein extraction.

The collected test sample is processed in an extraction/homogenization buffer. In some aspects, the extraction buffer is stored in the homogenization chamber 321 and may be mixed with the test sample by the homogenization rotor 340. In other aspects, the extraction buffer may be released into the homogenization chamber 321 from another separate storage chamber. The test sample and the extraction buffer will be mixed together by the homogenization rotor 340 and the sample being homogenized. In some embodiments, the extraction buffer is preloaded with a detection agent (e.g., SPN), thereby permitting the extracted molecule of interest from the test sample to interact with the detection agent.

The extraction buffer may be universal target extraction buffer that can retrieve enough target proteins from any test sample and be optimized for maximizing protein extraction. In some embodiments, the formulation of the universal protein extraction buffer can extract the protein at room temperature and in minimal time (less than 1 min). The same buffer may be used during food sampling, homogenization, and filtering. The extraction buffer may be PBS based buffer containing 10%, 20% or 40% ethanol, or Tris based buffer containing Tris base pH8.0, 5 mM MEDTA and 20% ethanol, or a modified PBS or Tris buffer. In some examples, the buffer may be a HEPES based buffer. Some examples of modified PBS buffers may include: P+ buffer and K buffer. Some examples of Tris based buffers may include Buffer A+, Buffer A, B, C, D, E, and Buffer T. As a non-limiting example, the extraction buffer may include 20 mM EPPS, 2% PEG 8000, 2% F-127 (Pluronic), 0.2% Brij-58 (pH8.4). In some embodiments, the extraction buffer may be optimized for increasing protein extraction. A detailed description of each modified buffer is disclosed in the PCT Patent Application No.: PCT/US2014/062656; the content of which is incorporated herein by reference in its entirety.

In accordance with the present disclosure, MgCl₂ is added after the sample is homogenized. In some embodiments, MgCl₂ solution (e.g., 30 μL of 1M MgCl₂ solution) is added to the homogenization chamber (e.g., 321 in FIG. 3F) after the sample homogenization.

In other embodiments, solid MgCl₂ formulations may be used in replacement of the addition of MgCl₂ solution during the reaction. The solid formulation may be provided as a MgCl₂ lyophilized pellet in the homogenization chamber (e.g., 321 in FIG. 3F) which is dissolved by the homogenate after filtration, or a filter component deposited or layered in the filter (e.g., the filter membrane 420 in FIG. 4A and the filter assembly 325 in FIG. 4A and FIG. 6D) that is dissolved by the homogenate during the filtration, or a MgCl₂ film deposited on the inner surface of the homogenization chamber 321), or MgCl₂ containing lyophilized beads stored in the filtrate chamber (e.g., the filtrate chamber 322) or on a separate support. In the context of the filter assembly 325, the cotton layer filter of the depth filter (e.g., 412) may be impregnated with the MgCl₂ formulation. Regardless of the formulations, MgCl₂ will dissolve in less than 1 minute, preferably in less than 30 seconds, to be contacted with the processed sample homogenate. MgCl₂ may dissolve in about 10 seconds, or about 15 seconds, or about 20 seconds, or about 25 seconds, or about 30 seconds. The solid formulation will release MgCl₂ within this short period of time to reach to a final concentration of 30 mM. In some aspects, the solid MgCl₂ formulation may not break up into powder.

The volume of the extraction buffer may be from 0.5 mL to 3.0 mL. In some embodiments, the volume of the extraction buffer may be 0.5 mL, 1.0 mL, 1.5 mL, 2.0 mL, 2.5 mL, or 3.0 mL. The volume has been determined to be efficient and repeatable over time and in different food matrices.

In accordance with the present disclosure, the test sample is homogenized and processed using the homogenization assembly that has been optimized with high speed homogenization for maximally processing the test sample.

In some aspects of the disclosure, a filtering mechanism may be linked to the homogenizer. The homogenized sample solution is then driven to flow through a filter in a process to further extract allergen proteins and remove particles that may interfere with the flow and optical measurements during the test, lowering the amount of other molecules extracted from the test sample. The filtration step may further achieve uniform viscosity of the sample to control fluidics during the assay. In the context that DNA glass chips are used as detection sensors, the filtration may remove fats and emulsifiers that may adhere to the chip and interfere with the optical measurements during the test. In some embodiments, a filter membrane such as cell strainer from CORNING (CORNING, NY, USA) or similar custom embodiment may be connected to the homogenizer. The filtering process may be a multi-stage arrangement with different pore sizes from first filter to second, or to the third. The filtering process may be adjusted and optimized depending on food matrices being tested. As a non-limiting example, a filter assembly with a small pore size may be used to capture particles and to absorb large volumes of liquid when processing dry foods, therefore, longer times and higher pressures may be used during the filtration. In another example, bulk filtration may be implemented to absorb fat and emulsifiers when processing fatty foods. The filtration may further facilitate to remove fluorescence haze or particles from fluorescence foods, which will interfere with the optical measurements.

The filter may be a simple membrane filter, or an assembly composed of a combination of filter materials such as PET, cotton, and sand, etc. In some embodiments, the homogenized sample may be filtered through a filter membrane, or a filter assembly. e.g., the filter assembly 325 in FIG. 4A.

In some aspects of the present disclosure, the sampling procedure may reach effective protein extraction in less than 1 minute. In one aspect, speed of digestion may be less than 2 minutes including food pickup, digestion, and readout. Approximately, the procedure may last 15 seconds, 30 seconds, 45 seconds, 50 seconds, 55 seconds, 1 minute or 2 minutes.

Extracted allergen proteins may be mixed with one or more detection agents that are specific to one or more allergens of interest. The interaction between allergen protein extraction and detection agents will generate a detectable signal which indicates the presence, or absence or the amount of one or more allergens in the test sample. As used herein, the term “detection agent” or “allergen detection agent” refers to any molecule which is capable of, or does, interact with and/or bind to one or more allergens in a way that allows detection of such allergen in a sample. The detection agent may be a protein-based agent such as antibody, a nucleic acid-based agent, or a small molecule.

In some embodiments, the detection agent is a nucleic acid molecule based signaling polynucleotide (SPN). The SPN comprises a core nucleic acid sequence that binds to a target allergen protein with high specificity and affinity. The SPN may be derived from an aptamer selected by a SELEX method. As used herein, the term “aptamer” refers to a nucleic acid species that has been engineered through repeated rounds of in vitro selection or equivalently, SELEX (systematic evolution of ligands by exponential enrichment) to bind to various molecular targets such as small molecules, proteins, nucleic acids, and even cells, tissues and organisms. The binding specificity and high affinity to target molecules, the sensitivity and reproductively at ambient temperature, the relatively low production cost, and the possibility to develop an aptamer core sequence that can recognize any protein, ensure an effective but simple detection assay.

In accordance with the present disclosure, SPNs that can be used as detection agents may be aptamers specific to a common allergen such as peanut, tree-nut, fish, gluten, milk and egg. For example, the detection agent may be the aptamers or SPNs described in applicants' relevant PCT application publication Nos. WO2015066027, WO2016176203, WO2017160616 and WO2018089391; and U.S. Provisional Application No. 62/714,102 filed Aug. 3, 2018; the contents of each of which are incorporated herein by reference in their entirety.

In some embodiments, the detection agent (e.g., SPN) may be labeled with a fluorescence marker. The fluorescence marker, fluorophore may suitably have an excitation maximum in the range of 200 to 700 nm, while the emission maximum may be in the range of 300 to 800 nm. The fluorophore may further have a fluorescence relaxation time in the range of 1-7 nanoseconds, preferably 3-5 nanoseconds. As non-limiting examples, a fluorophore that can be probed at one terminus of a SPN may include derivatives of boron-dipyrromethene (BODIPY, e.g., BODIPY TMR dye; BODIPY FL dye), fluorescein including derivatives thereof, rhodamine including derivatives thereof, dansyls including derivatives thereof (e.g. dansyl cadaverine), texas red, eosin, cyanine dyes, indocarbocyanine, oxacarbocyanine, thiacarbocyanine, merocyanine, squaraines and derivatives seta, setau, and square dyes, naphthalene and derivatives thereof, coumarnn and derivatives thereof, pyridyloxazole, nitrobenzoxadiazole, benzoxadiazole, anthraquinones, pyrene and derivatives thereof, oxazine and derivatives, nile red, nile blue, cresyl violet, oxazine 170, proflavin, acridine orange, acridine yellow, auramine, crystal violet, malachite green, porphin, phthalocyanine, bilirubin, tetramethylrhodamine, hydroxycoumarin, aminocoumarin; methoxycoumarin, cascade blue, pacific blue, pacific orange. NBD, r-phycoerythrin (PE), red 613; perCP, trured; fluorX, Cy2, Cy3, Cy5 and Cy7, TRITC, X-rhodamine, lissamine rhodamine B, allophycocyanin (APC) and Alexa fluor dyes (e.g., Alexa Fluo 488, Alexa Fluo 500, Alexa Fluo 514, Alexa Fluo 532, Alexa Fluo 546, Alexa Fluo 555, Alexa Fluo 568, Alexa Fluo 594, Alexa Fluo 610, Alexa Fluo 633, Alexa Fluo 637, Alexa Fluo 647, Alexa Fluo 660, Alexa Fluo 680, and Alexa Fluo 700).

In one example, the SPN is labeled with Cy5 at the 5 end of the SPN sequence. In another example, the SPN is labeled with Alexa Fluo 647 at the one end of the SPN sequence.

In some embodiments, the SPN specific to an allergen of interest may be pre-stored in the extraction/homogenization buffer in the homogenization chamber 321 (FIGS. 3B and 3F). The extracted allergen protein, if present in the test sample, will bind to the SPN, forming a protein:SPN complex. This protein:SPN complex can be detected by a detection sensor during a process of the test.

In some embodiments, detection agents for eight major food allergens (i.e. wheat, egg, milk, peanuts, tree nuts, fish, shellfish, and soy) may be provided as disposables. In one aspect, constructs of the detection agents may be stored with MgCl₂, or buffer doped with KCl. MgCl₂ keeps constructs closed tightly, while KCl opens them slightly for bonding.

In some embodiments, the detection sensor is a nucleic acid printed solid substrate. As used herein, the term “detection sensor” refers to an instrument that can capture a reaction signal, i.e. the reaction signal derived from the binding of allergen proteins and detection agents, measure a quantity and/or a quality of a target, and convert the measurement to a signal that can be measured digitally.

In some embodiments, the detection sensor is a solid substrate, such as a glass chip, coated with nucleic acid molecules (as referred to herein as nucleic acid chip or DNA chip). For example, the detection sensor may be the glass chip 333 inserted into the reaction chamber 331 of the present disclosure or a chipannel 710 in the test cup 300 (FIG. 7A). The detection sensor may also be a separate glass chip, for example, prepared from glass wafer and soda glass, or a microwell, or an acrylic glass, or a microchip, or a plastic chip made of COC (cyclic olefin copolymer) and COP (cyclo-olefin polymer), or a membrane like substrate (e.g., nitrocellulose), of which the surface is coated with nucleic acid molecules.

In some embodiments, the nucleic acid coated chip may comprise at least one reaction panel and at least two control panels. The reaction panel is printed with nucleic acid probes that hybridize to the SPN. As used herein, the term “nucleic acid probe” refers to a short oligonucleotide comprising a nucleic acid sequence complementary to the nucleic acid sequence of a SPN. The short complementary sequence of the probe can hybridize to the free SPN. When the SPN is not bound by a target allergen, the SPN can be anchored to the probe through hybridization. When the SPN bind to a target allergen to form a protein:SPN complex, the protein:SPN complex prevents the hybridization between the SPN and its nucleic acid probe.

In some examples, the probe comprises a short nucleic acid sequence that is complementary to the sequence of the 3′ end of the SPN that specifically binds to a target allergen protein. In this context, the SPN specific to the target allergen protein is provided in the extraction/homogenization buffer. When the sample is processed in the homogenization chamber 321, the target allergen, if present in the test sample, will bind to the SPN, and form a protein:SPN complex. When the sample solution flows to the detection sensor. e.g., the DNA chip 333 in the reaction chamber 331 (FIG. 3B) or the chipannel 710 (FIG. 7A), the bound allergen protein prevents the SPN from hybridizing to the complementary SPN probes on the chip surface. The protein:SPN complex is washed off and no fluorescence signal is detected. In the absence of the target allergen proteins in the test sample, the free SPN will bind to the complementary SPN probes on the chip surface. A fluorescence signal will be detected from the reaction panel (as shown in FIGS. 13A and 13B).

In some embodiments, the detection sensor, e.g., nucleic acid printed chip, further comprises at least two control panels. The control panels are printed with nucleic acid molecules that do not bind to a SPN or a protein (referred herein as “control nucleic acid molecules”). In some examples, the control nucleic acid molecules are labeled with a fluorescence marker.

In some embodiments, nucleic acid probes may be printed to a reaction panel at the center of a glass chip (“unknown”) and control nucleic acid molecules may be printed to the two control panels at each side of the reaction panel on the glass chip, as illustrated in FIG. 13A.

In some embodiments, the nucleic acid chip (DNA chip) may be prepared by any known DNA printing technologies known in the art. In some embodiments, the DNA chip may be prepared by using single spot pipetting to pipette nucleic acid solution onto the glass chip, or by stamping with a wet PDMS stamp comprising a nucleic acid probe solution followed by pressing the stamp against the glass slide, or by flow with microfluidic incubation chambers.

As a non-limiting example, a glass wafer can be laser cut to produce 10×10 mm glass “chips”. Each chip contains three panels; one reaction panel (i.e. the “unknown” area in the chip demonstrated in FIG. 13A) that is flanked by two control panels (FIG. 13A). The reaction panel contains covalently bound short complementary nucleic acid probes to which SPNs specific to an allergen protein bind. The SPNs are derived from aptamers and modified to contain a CY5 fluorophore. In the absence of the target allergen protein, SPNs are free to bind to the probes in the reaction panel, resulting in a high fluorescence signal. In the presence of the target allergen protein, the SPN: probe hybridizing interface is occluded by the binding of the target protein to the SPNs, thereby resulting in a decrease in fluorescence signal on the reaction panel. In a detection assay, the reaction panel of the chip faces a small reaction chamber (e.g. the reaction chamber 331) flanked by an inlet and outlet channel (e.g., 336 in FIG. 3H) of the cartridge (e.g., the cup 300). During food homogenization, the SPN in the extraction buffer binds to the target allergen if it is present in the sample forming a protein:SPN complex. The processed sample solution including the protein:SPN complex enters the reaction chamber 331 via the inlet, through fluidic movement driven by a vacuum pump. The solution then exits into a waste chamber 323 via the outlet channel. After exposure to the sample, the reaction panel is then washed, revealing a fluorescence signal with an intensity correlated to the target allergen concentration.

In some embodiments, the wash buffer is optimized to improve wash efficiency, increasing baseline signal and decreasing non-specific binding. As a non-limiting example, the wash buffer may be an optimized PPB buffer, including pluronic F-127 (e.g., 2% w/v), PEG-8000 (2% w/v). Brij 58 (e.g., 0.2% w/v) and EPPS (e.g., 20 mM), pH8.4.

In accordance with the present disclosure, the two control panels are constantly bright areas on the chip sensor that produce a constant signal as background signals 1301 and 1302 (FIG. 13B). In addition, the two control panels compensate for laser illumination and/or disposable cartridge misalignment. If the cartridge is perfectly aligned, then the fluorescence background signals 1301 and 1302 would be equal (as shown in FIG. 13B). If the measured control signals are not equal, then a look-up table of correction factors will be used to correct the unknown signal as a function of cartridge/laser misalignment. The final measurement is a comparison of the signal 1303 of the unknown test area against the signal levels of the control areas. The comparison level may be one of the lot-specific parameters for the test.

Food samples with high background fluorescence measurements from the reaction area may produce a false negative result. A verification method may be provided to adjust the process.

The final fluorescence measurement of the reaction panel, after being compared to the controls and any lot specific parameters may be analyzed and a report of the result may be provided.

Accordingly, the light absorption and light scattering signals may also be measured at the baseline level, before and/or after the injection of the processed food sample. These measurements will provide additional parameters to adjust the detection assay. For example, such signals may be used to look for residual food in the reaction chamber 331 after wash.

In addition to the parameters discussed above, one or more other lot-specific parameters may also be measured. The optimization of the parameters, for example, may minimize the disparity in the control and unknown signal levels for the chips.

In some embodiments, the monitoring process may be automatic and is controlled by a software application. Evaluation of the DNA chip and test sample, the washing process and the final signal measurement may be monitored during the detection assay.

Allergen families that can be detected using the detection system and device described herein include allergens from foods, the environment or from non-human proteins such as domestic pet dander. Food allergens include, but are not limited to proteins in legumes such as peanuts, peas, lentils and beans, as well as the legume-related plant lupin, tree nuts such as almond, cashew, walnut, Brazil nut, filbert/hazelnut, pecan, pistachio, beechnut, butternut, chestnut, chinquapin nut, coconut, ginkgo nut, lychee nut, macadamia nut, nangai nut and pine nut, egg, fish, shellfish such as crab, crawfish, lobster, shrimp and prawns, mollusks such as clams, oysters, mussels and scallops, milk, soy, wheat, gluten, corn, meat such as beef, pork, mutton and chicken, gelatin, sulphite, seeds such as sesame, sunflower and poppy seeds, and spices such as coriander, garlic and mustard, fruits, vegetables such as celery, and rice. The allergen may be present in a flour or meal, or in any format of products. For example, the seeds from plants, such as lupin, sunflower or poppy can be used in foods such as seeded bread or can be ground to make flour to be used in making bread or pastries.

In other embodiments, a clinical target may be detected using the present system. As used herein, the term “clinical target” refers to a molecule of interest that is clinically relevant, e.g., a diagnostic mark of a disease, an indicator of a treatment, a prognostic marker, etc. Samples may be a biological sample, such as saliva, blood, serum, plasma, urine and stool. Samples may be a cell culture medium.

### Applications

The detection systems, devices and methods described herein contemplate the use of nucleic acid-based detector molecules such as aptamers for detection of allergens in food samples. The portable devices allow a user to test the presence or absence of one or more allergens in food samples. Allergen families that can be detected using the device described herein include allergens from legumes such as peanuts, tree nuts, eggs, milk, soy, spices, seeds, fish, shellfish, wheat gluten, rice, fruits and vegetables. The allergen may be present in a flour or meal. The device is capable of confirming the presence or absence of these allergens as well as quantifying the amounts of these allergens.

Peanut allergy affects a significant portion of the population and with most fatal food reactions occurring outside the home. Recent epidemiological studies have shown that up to 2.2% of the population has an allergy to peanut, and the majority of fatal food reactions occur upon consumption of food outside of the home. The present assay will empower consumers to easily and quickly assess the presence of peanut allergens in foods before eating to help avoid and alleviate anxiety associated with accidental exposure, related health risks and costs, as well as emotional burden. This type of technology has the potential to improve lives and decrease risk for children and families. The novel aptamer-based protein detection method is robust across a wide variety of food matrices and sensitive to peanut at concentrations as low as 50 ppm (50 parts per million, or mg/L of peanut flour or approximately 12.5 ppm peanut protein).

As explained in Example 3 below, the system accurately detects peanut in raw ingredients. The USDA, the Association of Official Agricultural Chemists (AOAC), the Food Allergy Research and Resource Program (FARRP), and the International Association for Monitoring and Quality Assurance in the Total Food Chain (MoniQA) have created standards by which allergen detection may be rated. The system, as shown in Table 4 below, has shown accuracy in peanut detection across a wide range of components. The results of the system significantly exceeds market standards as demonstrated in 45 foods and 14 categories: Baked goods (cookies, grain breads, energy bars, cakes cupcakes, toppings and filings), chocolate, ice cream, salad dressing, sauces, noodles and pasta, spices, soups and chili, cereal and granola. Asian food, honey, non-chocolate candy, snacks, and others.

The system includes science and technology controls with built-in precision and sensitivity controls with both the assay and any hardware feedback look being responsive to the user and consumer touchpoints. An application or other software control (such as on a smartphone) provides a detailed tutorial on usage of the system and, in conjunction with a guide and website, illustrates consumer use recommendations. Customer support ensures support for the product and user. The system follows all performance testing methods and recommendations from AOAC and other governing bodies related to allergy and allergen detection as well as publish independent lab verification. The system may include a carrying case to hold the device, extra cartridges or pods, as well as other related devices such as an epinephrine, diphenhydramine tables, or other emergency medicines. The application may help decrease the risk of encountering an allergen. The Application may remind the user to “communicate” and remind the user to ask a server in a restaurant about allergens in the food. The Application can “remind” the user to look and remind the user to read the ingredient list carefully and visually inspect the food. The Application may remind the user to “ask again,” and ask the server about allergens in the food when the plate is given. The Application may remind the user to “review.” and confirm that the user understands the allergen test and that the test is set at a level of 12.5 ppm. The Application may remind the user to have “emergency medicine” on hand, just in case.

The system, as a whole, has an ease of functionality which ensures user success. The user first collects a food sample and can weigh the sample on an integrated scale in the lid of the detection device as discussed above. The system can alert the user if additional food is needed and will indicate that the pd should be removed and to add more food. The system will also indicate if too much food has been added to the pod and indicate that a new pod should be used. Once enough food has been added and processed, the system ensures that the resultant images are analyzable. After an image analysis has been generated, the system determines if the image meets acceptance criteria. After this point, the system provides results-whether the allergen (a peanut, etc.) is detected or not detected. A control panel in the system (i.e. on a smartphone) indicates how the food impacts the assay regardless of the presence of peanut and can be used to calibrate the test panel system. An algorithm of the system incorporates the intensity of the test and control panel, in addition to the time of reaction, background, and pressure curves to yield the most accurate output. The algorithm utilizes a stringent penalty function to skew output away from false negatives.

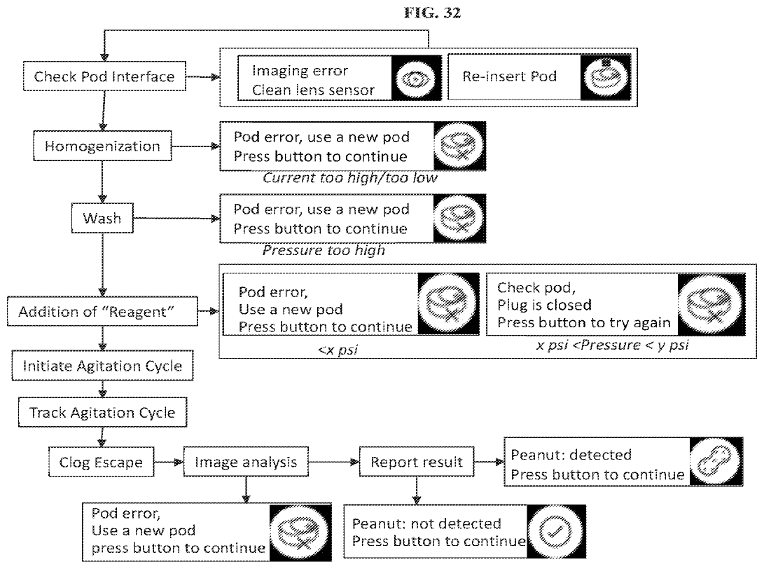

The algorithm in the system may be represented by FIG. 32. The pod is installed in the device and monitored for optical and fluidic connection. The rotor or homogenizer speed is monitored during food mastication or processing. The flow during the was and mixing step is monitored via an on-board pressure transducer. Minimal imaging standards are assessed prior to result reporting.

In a broad concept, the detection systems, devices and methods described herein may be used for detection of any protein content in a sample in a large variety of applications in addition to food safety, such as, for example, medical diagnosis of diseases in civilian and battlefield settings, environmental monitoring/control and military use for the detection of biological weapons. In even broad applications, the detection systems, devices, and methods of the present disclosure may be used to detect any biomolecules to which nucleic acid-based detector molecules bind. As some non-limiting examples, the detection systems, devices and methods may be used on the spot detection of cancer markers, in-field diagnostics (exposure the chemical agents, traumatic head injuries etc.), third-world applications (TB, HIV tests etc.), emergency care (stroke markers, head injury etc.) and many others.

As another non-limiting example, the detection systems, devices, and methods of the present disclosure can detect and identify pathogenic microorganisms in a sample. Pathogens that can be detected include bacteria, yeasts, fungi, viruses and virus-like organisms. Pathogens cause diseases in animals and plants; contaminate food, water, soil, or other sources; or is used as biological agents in military fields. The device is capable of detecting and identifying pathogens.

Another important application includes the use of the detection systems, devices, and methods of the present disclosure for medical care, for example, to diagnose a disease, to stage a disease progression and to monitor a response to a certain treatment. As a non-limiting example, the detection device of the present disclosure may be used to test the presence or absence, or the amount of a biomarker associated with a disease (e.g. cancer) to predict a disease or disease progression. The detection systems, devices and methods of the present disclosure are constructed to analyze a small amount of test sample and can be implemented by a user without extensive laboratory training.

Other expanded applications outside of the field of food safety include in-field use by military organizations, testing of antibiotics and biological drugs, environmental testing of products such as pesticides and fertilizers, testing of dietary supplements and various food components and additives prepared in bulk such as caffeine and nicotine, as well as testing of clinical samples such as saliva, skin and blood to determine if an individual has been exposed to significant levels of an individual allergen.

### EQUIVALENTS AND SCOPE

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

A number of possible alternative features are introduced during the course of this description. It is to be understood that, according to the knowledge and judgment of persons skilled in the art, such alternative features may be substituted in various combinations to arrive at different embodiments of the present disclosure.

Any patent, publication, internet site, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

### EXAMPLES

### Example 1: Testing Filter Materials and Filtering Efficiency

Various filter materials and their combinations are tested for filtering efficiency and effect on signal measurement, for example, the loss of detection agents (SPNs). Commercially available filter materials such as membranes (PES, glass fiber, PET, PVDF, etc.), cotton, sand, mesh, and silica are tested.

A filter including a combination of different filter materials is assembled. In one example, the filter assembly is composed of cotton and glass filter with a pore size of 1 μm. The cotton depth filter and paper filter are constructed to filter the sample sequentially. The filter assembly is tested for filtering different food matrices. The recovery of proteins and SPNs during the filtering process is measured. Various cotton volumes are used to construct the depth filters and the cotton depth filters are combined with membrane filters. These filter assemblies are tested for filtration efficiency and SPN recovery. In one study, 0.5 g of a food sample is collected and homogenized in 5 ml EPPS buffer (pH 8.4) (Tween 0.1%) and the homogenized food sample is incubated with 5 nM SPNs (signaling polynucleotides) labeled with Cy5 that is specific to an allergen protein. After incubation, a portion of the mixture is run through the filter assemblies and the recovery of proteins and SPNs is measured and compared with the pre-filtering measurements.

The filters are further tested and optimized to ensure efficiency of filtration and avoidance of significant SPN loss. In addition to testing different filter materials and their combinations, other parameters such as pore sizes, filtering areas (e.g., surface area/diameter, height of the depth filter), filtering volumes, filtration time and pressure required to drive the filtering process, etc., are also tested and optimized for various food matrices.

In one study, bleached cotton balls are used to assemble the depth filters with different filter volumes. Cotton filters with different ratios of width (i.e. diameter) and height are constructed; each model has a ratio of width and height ranging from about 1:30 to about 1:5. The cotton depth filters are then tested for filtration efficiency with different food masses and buffer volumes. In another study, these model cotton filters are assembled together with a PET membrane filter with 1 μm pore size and about 20 mm² filtrating area. Various food samples are homogenized and filtered through each filter assembly using different volumes of buffer. The filtrates are collected and the percentage of recovery is compared for each condition.

In another study, food samples are spiked with or without 50 ppm peanut. The spiked samples are homogenized, for example using the rotor 340 (e.g., as illustrated in FIGS. 3B and 3C) and the extractions are mixed with SPNs that specifically bind to peanut allergen. The SPN contains a Cy5 label at the 5′ end of the sequence. The mixture is filtered through a depth filter (e.g., a depth filter made of cotton) and a membrane filter (pore size: 1 μm). Fluorescence signals are measured and compared with the measurements of the pre-filtered mixture.

In separate studies, several parameters of each filter assembly are tested and measured including the pressure and time required for filtering, protein, and nucleic acid binding, washing efficiency and assay compatibility and sensitivity. The assay compatibility is measured as the baseline intensity.

### Example 2: MgCl₂ Formulations

Several solid MgCl₂ formulations were tested to replace the addition of MgCl₂ solution after the sample homogenization in extraction buffer. The following characteristics of each formulation tested are evaluated: (1) the time to dissolve; (2) the final concentration of dissolved MgCl₂; (3) the effect of additives in the formulations on the detection assay; (4) no agitation required to dissolve; and (5) no breakup into powder and not blocking the outlet of the homogenization chamber.

### Lyophilized MgCl₂ Formulation

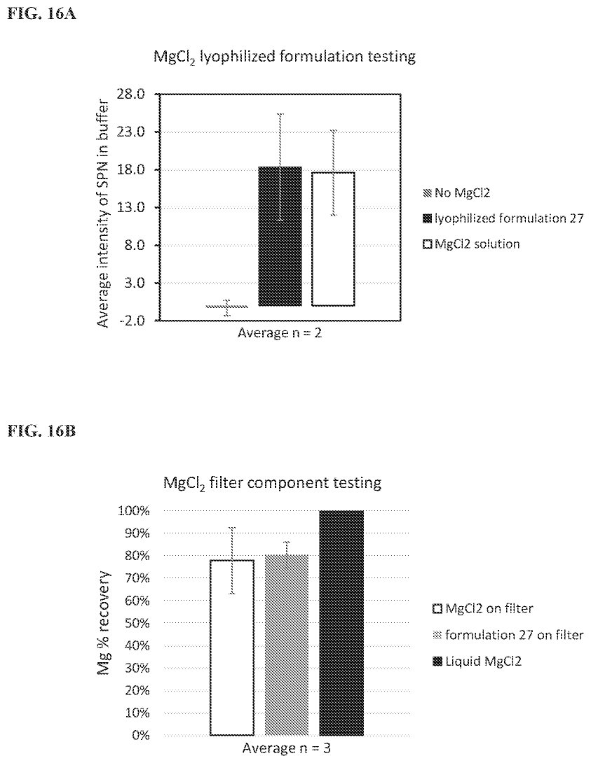

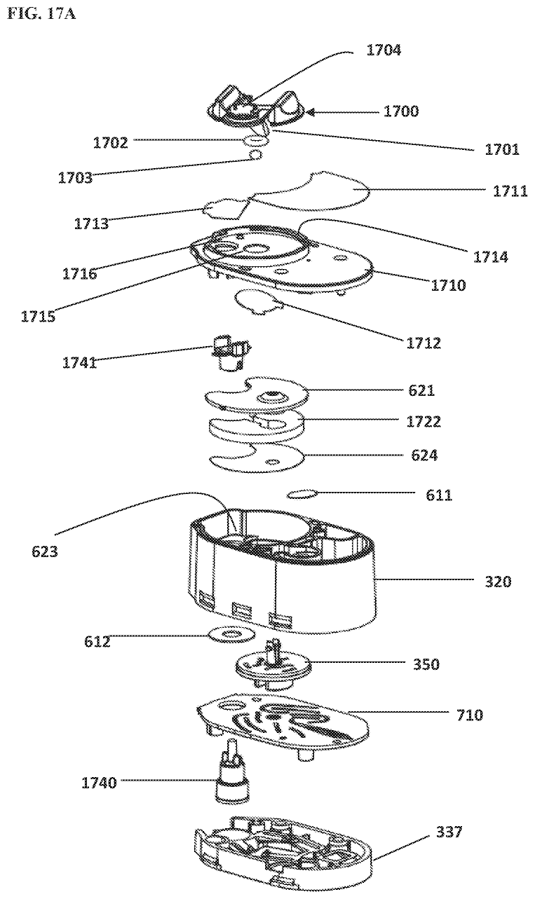

34 MgCl₂ formulations were lyophilized in 1.5 mL Eppendorf tubes and tested for dissolution time, mechanical stability, exposure to the extraction buffer for 10 seconds without agitation, and other features. 2 formulations are rapidly dissolving and do not form powder. Several MgCl₂ formulations were exposed to the extraction buffer for 10 seconds without agitation and the magnesium content in the recovered buffer was determined by a BioVision Magnesium assay and the assay as described herein. The assay results indicate that the lyophilized MgCl₂ formulation comprising maltodextrin and hydroxyethylcellulose (HEC) (Table 1) gives the highest intensity of SPNs in buffer as shown in FIG. 16A.

### MgCl₂ as a Filter Component

MgCl₂ formulations (Table 1) were deposited on a cotton filter and dried at 60° C. The extraction buffer was pulled through the cotton filter with 1 psi vacuum. The percentage of magnesium recovered in filtrate was measured by the BioVision colorimetric magnesium assay. The MgCl₂ formulation comprising maltodextrin and hydroxyethylcellulose (HEC) (Table 1) was compared with what was recovered in MgCl₂ solution and MgCl₂ on the filter (FIG. 16B).

### MgCl₂ as Film

10 different MgCl₂ formulations were deposited on polystyrene supports and cured. The dissolution time was measured and all formulations dissolved in 10 seconds. The results indicate that none of the formulations have a strong adhesion to the polystyrene support.

TABLE 1
Components of MgCl₂ formulations
Formulations containing 1.0% glycerol

| | | | |
|---|---|---|---|
| glycerol | 1.0% | PEG | 2.00% |
| | | PEG | 1.00% |
| | | PEG | 0.3% |
| | | PEG | 0.5% |
| | | glycine | 2.5% |
| | | sugar | 0.5% |
| | | maltodextrin | 0.5% |
| | | PEG | 0.3% |

Formulations containing 0.7% glycerol

| | | | |
|---|---|---|---|
| glycerol | 0.7% | PEG | 2.00% |
| | | PEG | 1.00% |
| | | PEG | 0.3% |
| | | PEG | 0.5% |
| | | glycine | 2.5% |
| | | sugar | 0.5% |
| | | maltodextrin | 0.5% |
| | | PEG | 0.3% |

Formulations containing 0.5% glycerol

| | | | |
|---|---|---|---|
| glycerol | 0.5% | PEG | 2.00% |
| | | PEG | 1.00% |
| | | PEG | 0.3% |
| | | PEG | 0.5% |
| | | glycine | 2.5% |
| | | sugar | 0.5% |
| | | maltodextrin | 0.5% |
| | | PEG | 0.3% |
| PEG | 2.0% | glycine | 2.5% |
| PEG | 5.0% | glycine | 2.5% |
| maltodextrin | 0.5% | HEC | 0.1% |

Based on the test results, several fast-dissolving solid MgCl₂ formulations are selected (as shown in Table 2). The dissolution time for the filter deposition is dependent on flow rate. When the fastest flow rate was tested, the solid formulation dissolved in 10 seconds (as shown in Table 2).

TABLE 2
Fast-dissolving and mechanically robust solid MgCl₂ formulations

| | Lyophilized | | |
|---|---|---|---|
| | pellet | Film | Incurred in filter |
| Leading formulation | 0.5% glycer- | 1% maltodex- | 1% maltodex- |
| | ol/0.5% su- | trin/0.1% | trin/0.1% |
| | crose | hydroxyethyl | hydroxyethyl |
| | | cellulose | cellulose |
| Time for resuspension | 12 Seconds | 16 seconds | 10 seconds |
| Stability following | − | + | N/A |
| agitation (vortex 1 | | | |
| minute) | | | |
| Mg recovery in 10 | 100% | 100% | 80% |
| seconds (compared to | | | |
| MgCl₂ solution) | | | |

### Example 3: Assay Validation

As discussed herein, the disclosure described the successful use of aptamer technology in a consumer device for the detection of peanut antigen in food. Detection of peanut protein was chosen due to the high population of people allergic to peanut (Arachis hypogaea). The present method addresses the need for simple, rapid antigen detection by designing a fluorophore-labeled aptamer-based assay for the detection of allergenic peanut proteins and have incorporated it into the easy to use, point-of-care device of the present disclosure that is suitable for consumer use. The assay is sensitive to peanut at concentrations as low as 50 ppm (50 parts per million, or mg/L of peanut flour or approximately 12.5 ppm peanut protein). This novel aptamer-based protein detection method is robust across a wide variety of food matrices.

This rapid and simple test approach was extended to demonstrate the strength and adaptability by also detecting gluten. Additional data demonstrate the potential of the approach to be adapted to other allergic antigens, such as gluten, and even serve molecular diagnostic purposes.

### Results

### Aptamer Selection

Five aptamers were initially chosen (P1-16, P1-10, PT-31, P2-8, and P2-18), based on the peanut-targeted SELEX pool, and underwent sequence modifications to improve tertiary structure formation using the predicted change in Gibbs free energy. To develop an aptamer-based assay with an optical readout, all five aptamers were conjugated with a Texas Red (TR) fluorophore on the 5′ end.

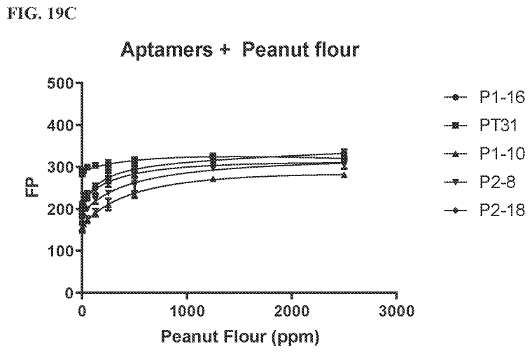

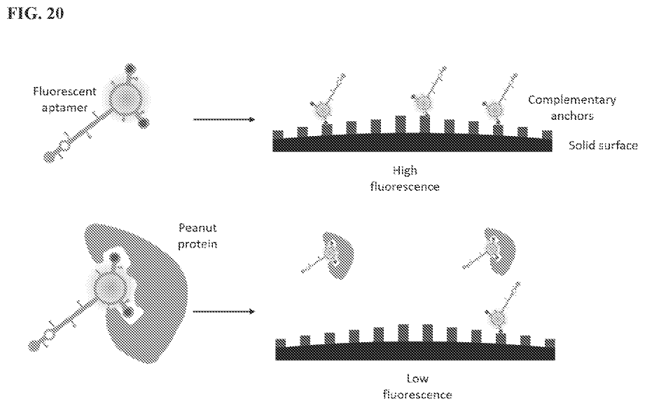

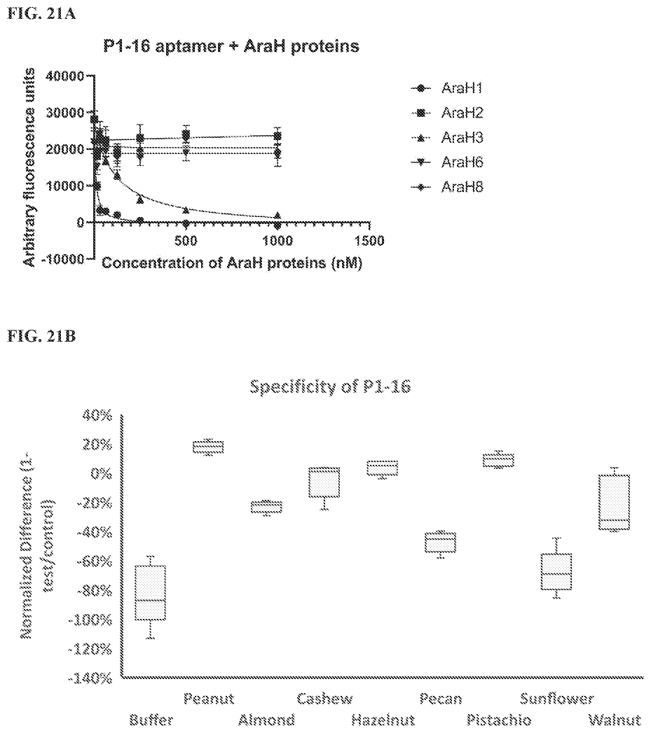

To determine the affinity of each optimized aptamer for Ara h 1, a major peanut allergen and the most abundant allergen in peanut (12-16% of the total protein content) 16.17, increasing amounts of purified unlabeled Ara h 1 were incubated with each aptamer and analyzed with fluorescence polarization (FP) to screen for binding affinity (FIG. 19A). The TR-labeled P1-16 aptamer yielded the highest affinity for Ara h 1 protein (Kd ˜54±5.5 nM, Table 3), followed by P2-18 and P2-18 with slightly lower affinities. FIGS. 19A-C show the determination of dissociation constants (Kds) for five peanut aptamers and targets. Five aptamers were incubated with increasing concentrations of target purified AraH1 protein to determine the Kd by fluorescence polarization. (A) Purified AraH1, (B) Peanut butter, (C) Peanut flour. Five independent replicates were tested, and fitting of the binding isotherm yielded Kd values shown in Table 3. Error bars represent the standard deviation of the mean.

TABLE 3
Summary of determination of dissociation constants (Kds).
Kd values shown are mean +/− standard error.

| | P1-16 | PT-31 | P1-10 | P2-8 | P2-18 |
|---|---|---|---|---|---|
| AraH1 protein | 54.4 ± | 238.1 ± | 69.2 ± | 265.8 ± | 104.7 ± |
| | 5.5 | 35.3 | 9.4 | 25.8 | 13.5 |
| Peanut butter | 141 ± | 311.3 ± | 544.7 ± | 650.8 ± | 386.5 ± |
| | 21.9 | 108.5 | 110.3 | 152.7 | 89.1 |
| Peanut flour | 144.3 ± | 791.7 ± | 557.2 ± | 466.1 ± | 383.1 ± |
| | 31.4 | 648.6 | 166.6 | 187.1 | 143.5 |

To determine whether the aptamers could detect the presence of Ara h 1 in processed peanut, the aptamers were incubated in commercially available peanut flour or peanut butter and measured FP. As observed with purified Ara h 1 protein, the TR-labeled P1-16 aptamer yielded a higher affinity for Ara h 1 in the complicated matrices of peanut butter and peanut flour (Kd ˜141±21.9 ppm, and 144±31.4 ppm, respectively) when compared to the other four aptamers (FIGS. 25B-C).

### Assay Design

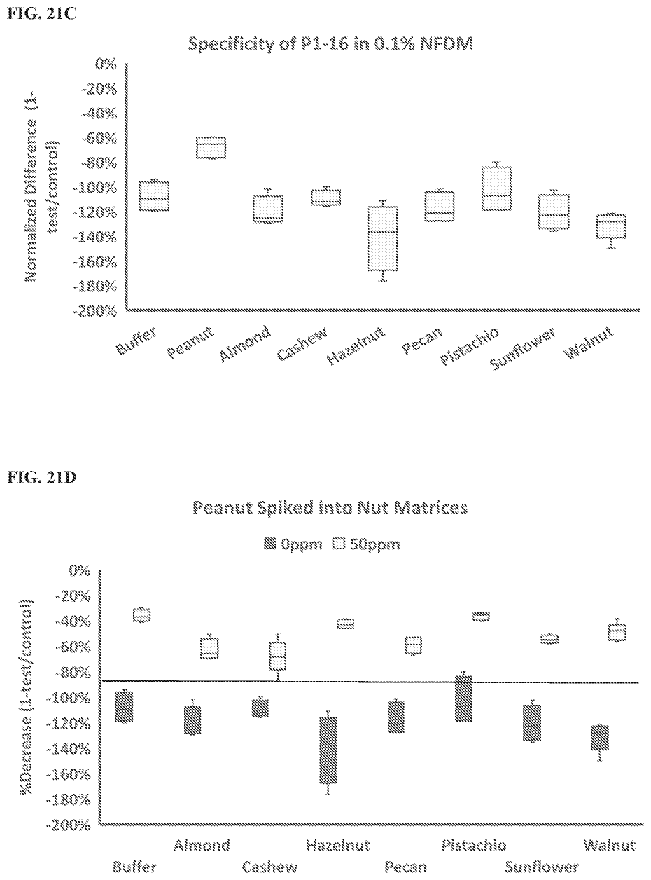

Foods were chosen in order to increase the testing data set to maintain sensitivity at 99% and decrease specificity to 93% and in order to shift the assay toward false positives. While FP is a sensitive approach to study interactions, it is also responsive to viscosity, temperature, and motion effects, and can be affected by auto-fluorescence of the test matrix. The matrix used in a study (blood, food, cells, etc.) can increase the viscosity and affect the tumbling of molecules in solution, thereby causing a potential increase in FP without the binding of two molecules occurring. Matrices can also contribute to total fluorescence intensity due to the intrinsic fluorescence of dyes, biological molecules, etc.; this can result in reduced sensitivity. To overcome these limitations, we designed a robust assay utilizing short complementary sequences (“anchors”) that are attached to a solid support (FIG. 20). In the assay, the fluorescently labeled aptamer is incubated with the food sample to be tested and, subsequently, to the immobilized anchor sequences. If the aptamer is bound to peanut antigen, it cannot bind to the anchor, and is removed during a subsequent washing step. High fluorescence detected on the support surface therefore signals the absence of peanut antigen (labeled aptamer binds the anchor), and low fluorescence occurs when peanut antigen is present (labeled aptamer is not bound to the anchor).

To select the best anchor sequence for the application, 40 short DNA sequences (anchors), complementary to various regions of the described aptamers, were covalently attached to an optically clear glass surface. They differed by oligonucleotide sequence, length, or composition/length of the linker (carbon atoms or poly-A tail). To reduce the probability of interference due to matrix auto-fluorescence, the aptamers were conjugated to Cyanine 5 (Cy5) rather than TR. After incubation of Cy5-labeled aptamers with homogenized peanut flour, the peanut flour-aptamer mix was added to wells containing the 40 complementary anchors immobilized to glass. After incubation and washing, we detected a decrease in Cy5 fluorescence associated with an anchor complementary to the P1-16 aptamer. Dilution experiments showed the signal was dependent on the concentration of peanut flour (FIGS. 25A-C) with sensitivity as low as 50 ppm. Interestingly, utilization of the immobilized anchor sequence without the poly-A tail resulted in less sensitivity and decreased baseline signal. These results were replicated when the anchor was extended with an additional six carbons, suggesting the positioning of the anchor relative to the glass surface is a key influence on aptamer binding. FIGS. 25A-C depict that CY5-N5 aptamer binds to gluten in a concentration dependent manner and in a variety of food matrices. GN5 aptamer was incubated in buffer spiked with increasing concentrations of gluten. In FIG. 25B shows commercially available foods (gluten versus gluten-free) were homogenized, filtered, then incubated with GN5. As described for the P1-16 aptamer, the samples were incubated with a chiplet spotted with a 10-oligonucleotide anchor complementary to sequence of GN5. Four replicates of each sample were tested. Error bars represent the standard deviation of the mean.

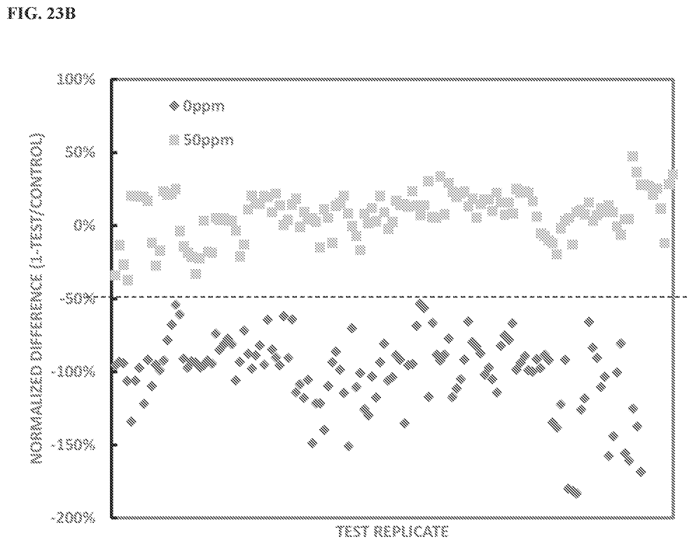

Functionality of the assay was confirmed by determining the specificity of the P1-16 aptamer to various Ara h proteins. FIGS. 23A-D depict specificity. (A) Alexa Fluor 647-labeled P1-16 aptamer binds to AraH1 and AraH3. Binding specificity was assessed by incubating purified AraH proteins, AraH1, AraH2, AraH3, AraH6, and AraH8, with AF647-P1-16 aptamer and testing with the benchtop assay. Curve fitting was performed using non-linear regression analysis. Four replicates were tested for each concentration with error bars representing the standard deviation of the mean. (B) P1-16 aptamer binds to peanut protein(s) preferentially to tree nuts. P1-16 is sensitive to tree nuts in a concentration-dependent manner. P1-16 aptamer was incubated in clarified peanut or tree nut flours blended in assay buffer. (C) 0.1% milk added to the buffer. (D) P1-16 aptamer was incubated with clarified tree nut homogenate at 50 ppm (or control buffer) and spiked with 0 or 50 ppm peanut flour. Four or five replicates were tested for each concentration. Consistent with the FP data, fluorescent intensity of P1-16 decreased with increasing concentrations of Ara h 1 protein and Ara h 3 but not Ara h 2. Ara h 6, or Ara h 8 proteins (FIG. 21A). The finding that P1-16 binds to both Ara h 1 and Ara h 3 is not surprising given that both of these proteins are members of the cupin superfamily with a root mean square deviation (r.m.s.d) of 2.4 Å when their crystal structures are aligned. Proteins of the cupin superfamily contain a conserved beta-barrel motif and include other tree nut allergens, such as those found in walnut (e.g., Jug r 2 protein) and hazelnut (e.g., Cor a 9 protein).

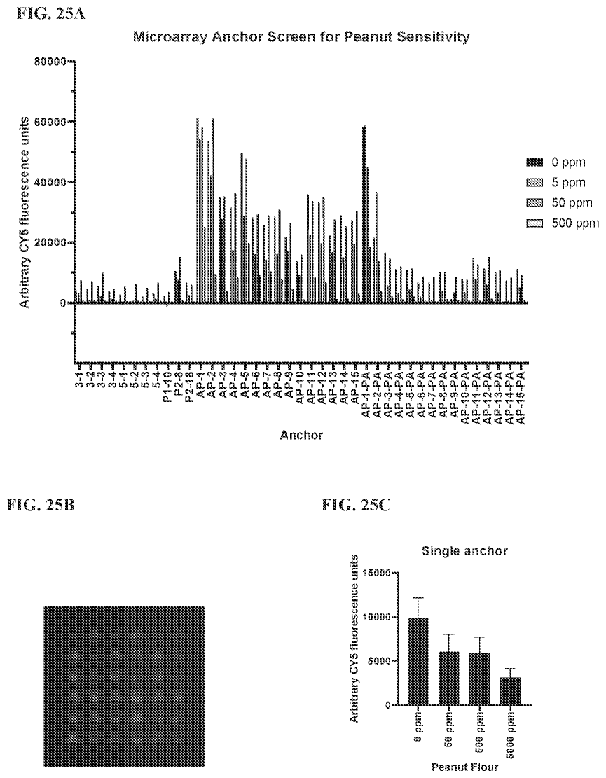

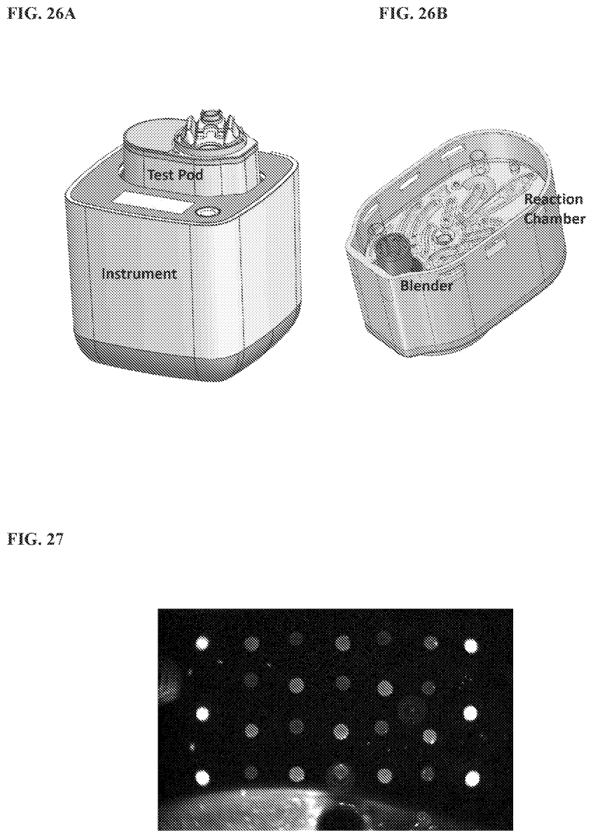

To transition the assay to a consumer-friendly tool to help allergic individuals manage their food consumption choices, we integrated the assay into a small single-use reaction capsule that is run on a durable instrument FIG. 26A. FIGS. 26A-B depict illustrations of the integrated assay test pod and instrument. Single-use test pod is driven by the durable instrument (left). Cutaway view of pod (right) shows area where food sample is homogenized and the reaction chamber containing the surface bound anchor sequences. We knew that for this assay to have a potential for commercial use, it would need to be fast, simple to use, compatible with solid food matrices, sensitive across different sample types, and exhibit a bright, stable signal. Thus, the device was designed to receive small food samples (0.1 g) in a capsule containing P1-16 and homogenization buffer (see Methods). The capsule then homogenizes samples, via a small blender, and passes the homogenate through a polyethylene terephthalate (PET) mesh filter to remove large particulates. The “cleaned” homogenate then flows through a reaction chamber using a propriety fluidic sequence (FIG. 26B), where the anchor sequences are bound. After rapid incubation (1-5 minutes, variable by sample), the aptamer containing homogenate is washed away, and the empty reaction chamber is imaged by a camera on the instrument. Proprietary algorithms detect and interpret the fluorescence from the remaining bound aptamer and produce a result “Peanut protein detected” or “Peanut protein not detected”.

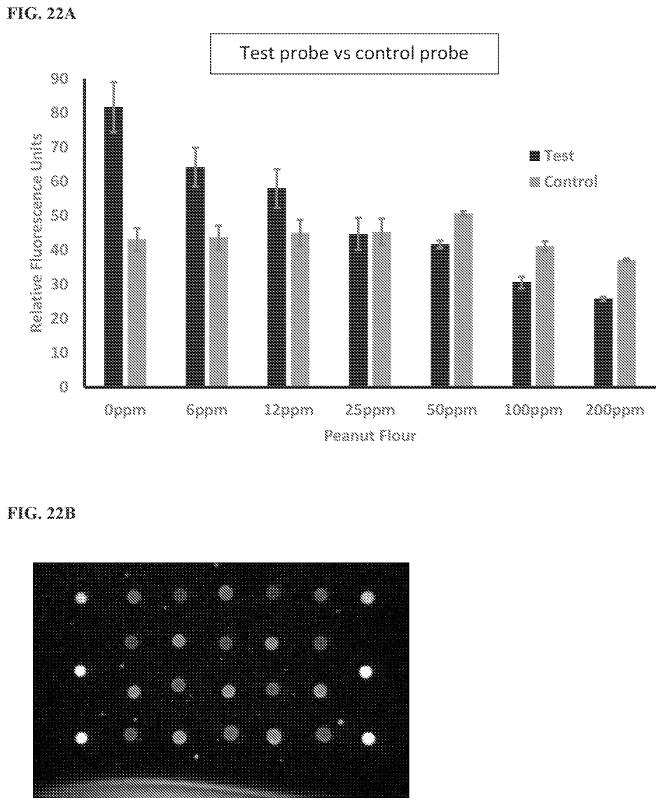

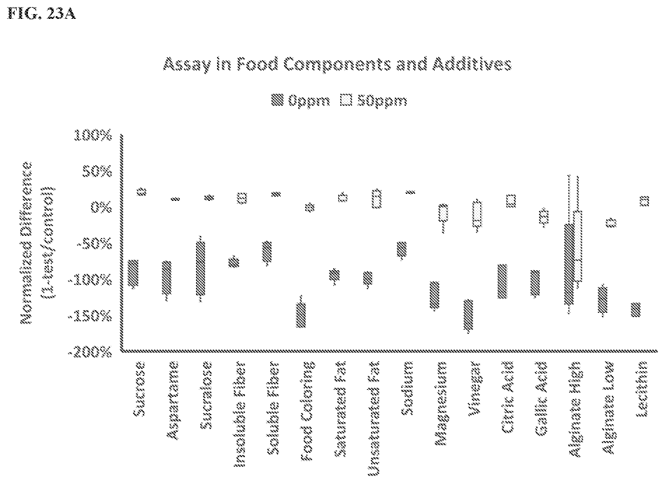

To improve robustness of the assay, we designed and tested approaches to normalize the fluorescent signal to an internal control. First, we searched for a control anchor sequence complimentary to a second region of the P1-16 aptamer whose binding would not be sensitive to peanut concentration. To do this, we revisited the forty sequences in our initial screen (FIG. 25). Sequences that were complementary or proximal to the covalent anchor or were sensitive to peanut were eliminated. Additional anchor sequences were designed to shift the control anchor sequence farther from the anchor-P1-16 binding site. Most anchors exhibited some reduction in fluorescence intensity in the presence of peanut; however, one anchor sequence exhibited only a modest non-significant decrease in intensity when peanut flour up to 200 ppm was introduced. These results suggested that this sequence may function as a matrix condition control (FIG. 22A). FIGS. 22A-B depict AF647-P1-16 aptamer binds to control anchor. (A) The comparison of P-16 aptamer binding to two different anchors (test and control) spotted on the same surface was assessed by incubating P1-16 aptamer with increasing concentrations of clarified peanut flour homogenate. Five replicates of each peanut flour concentration were tested. Error bars represent the standard error of the mean. (B) Representative image of Alexa Fluor 647-P1-16 aptamer bound to both the test spots (top left and alternating) and the control spots. The brighter spots on the left and right sides represent alignment markers for optical performance. We immobilized spots of the insensitive “control” anchors and peanut-sensitive “test” anchors in a checkerboard pattern (FIG. 22B) on the solid surface of the reaction capsule to compensate for debris or uneven flow and/or illumination of the reaction chamber (FIG. 27) and adjusted the detection algorithm to image only after a minimum fluorescence was reached in the control spots. FIG. 27 depicts an example of a poor image in which several spots cannot be used due to poor reaction flow (remaining fluid in bottom of image) and particulates of food debris (bright speckles). Poor spots are not considered in final analysis. The fluorescent intensities of the test and control spots were then averaged individually and the difference in intensities between the control and test was then normalized (1−intensity of test/intensity of control) to yield a single value comparable across a variety of food matrices.

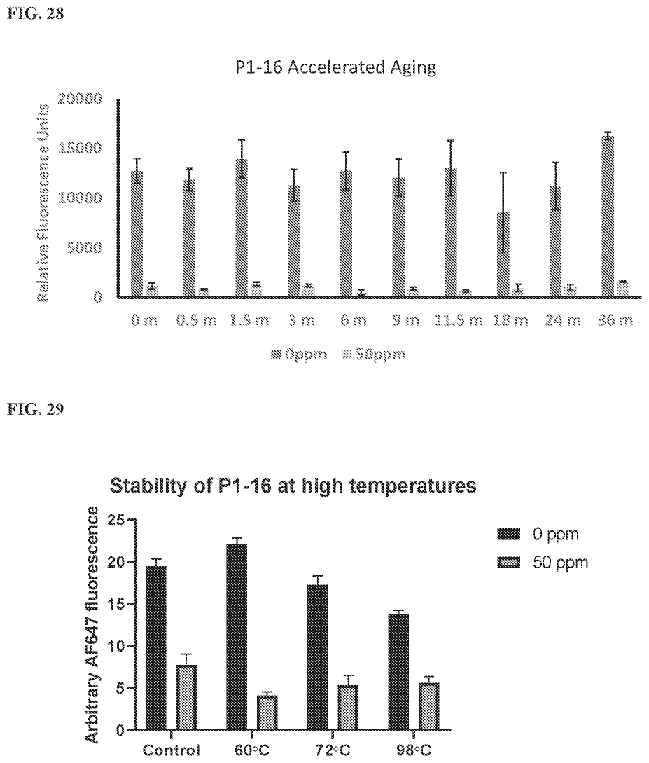

As a final step to achieve a brighter signal and improved image analysis, we switched the fluorophore from Cy5 to Alexa Fluor 647 (AF647)26. A significant concern with aptamers is stability of the three-dimensional conformation, both over time and at high temperatures. Accelerated aging studies on the AF647 modified P1-16 aptamer at 37 C (FIG. 28) showed that the aptamer retained its function over a period of at least three years (68 weeks in real time). FIG. 28 depicts AF647-P1-16 retains its sensitivity to peanut over an accelerated aging of 3 years. Time on x-axis is in months (m). To determine whether P1-16 was able to retain function when exposed to prolonged melting temperatures, we incubated the AF647 modified P1-16 aptamer in a thermocycler above its predicted melting temperature (Tm ˜75 C) and confirmed that function was retained (FIG. 29). FIG. 29 depicts that AF647-P1-16 retains the ability to bind peanut after exposure to high temperatures. AF647-p1-16 was incubated for 10 minutes at 60° C., 72° C., or 98° C. then cooled to 4° C. at a rate of 2 degrees/seconds. Sensitivity was assessed by incubating AF647-P1-16 aptamer with 50 ppm clarified peanut flour homogenate. Four replicates of the control sample and each temperature were tested. Error bars represent standard deviation of the mean.

### Assay Performance

As this assay was designed to detect peanut protein(s), we challenged the assay by testing the P1-16 aptamer against multiple types of tree nuts to gauge reactivity towards foods containing proteins of the cupin superfamily25. Commercially-available almond (Prunis dulcis), cashew (Anacardium occidentale), hazelnut (Corylus avellana), pecan (Carya illinoinensis), pistachio (Pistacia vera), sunflower (Helianthus annuus), and walnut (Juglans regia) flours were tested at 0 ppm and 50 ppm and compared to peanut flour. The normalized difference between the test and the control spots decreased in the presence of 50 ppm of the tree nuts tested (FIG. 21B), which could indicate significant cross-reactivity. To test whether the specificity of AF647-P1-16 for Ara h 1/Ara h 3 is greater compared to other cupin family proteins, we added 0.1% non-fat dry milk to the assay to serve as a nonspecific protein food matrix. The decrease of the test panel compared to the control panel was significantly greater in the presence of peanut when compared to the tree nuts tested in the presence of 0.1% milk (FIG. 21C). We further challenged this notion by performing a competition experiment by spiking peanut flour with the same concentration of tree nut flours to gauge whether peanut protein(s) could compete with tree nut protein(s) for P1-16 binding (FIG. 21D). The clear distinction in normalized difference between samples that contain peanut and those that do not suggested that P1-16 is responsive to peanut protein in the presence of tree nut.

A robust assay retains sensitivity regardless of the matrix analyzed, but it is infeasible to test every possible food. Therefore, we performed a guard study to investigate the effects of potentially high-risk food components and additives (e.g., fats, acid). Tests were conducted at the highest level typically seen in foods as reported by the USDA in the presence and absence of 50 ppm peanut flour (Table 4).

TABLE 4
Food components and additives tested in guard band studies.
Rightmost column lists an example food with the highest expected
amount of substance tested; detection of peanuts in

| | 0 | 12.5 | Amount | |
|---|---|---|---|---|
| | ppm | ppm | (relative | |
| Component/ | (de- | (de- | to pure | Example Food |
| Additive | crease) | crease) | component) | and Amount |
| Sucrose | −89% | 19% | 100% | Table Sugar (100%) |
| Aspartame | −97% | 10% | 4% | Equal Packet (4%) |
| Sucralose | −84% | 12% | 1% | Splenda |
| | | | | Packet (1%) |
| Insoluble Fiber | −78% | 12% | 90% | Corn Starch (90%) |
| (Corn Starch) | | | | |
| Soluble Fiber | −61% | 17% | 1% | Black |
| (Carbohydrates) | | | | Beans (10%)--- |
| Food Coloring | −150% | −2% | 1% | Trix Cereal (0.1%) |
| Saturated Fat | −94% | 11% | 100% | Coconut Oil (92%) |
| Unsaturated Fat | −99% | 11% | 100% | Olive Oil (86%) |
| Sodium | −59% | 19% | 2% | Cured ham (1.5%) |
| Magnesium | −125% | −7% | 1% | Pumpkin |
| | | | | seeds (0.5%) |
| White Vinegar | −152% | −12% | 25% | Traditional |
| | | | | vinaigrette (25%) |
| Citric Acid | −110% | 6% | 1% | Lemon juice (8%) |
| Gallic Acid | −102% | −14% | 0.1% | Red Wine (0.1%) |
| Alginate | −124% | −26% | 1% | Restructured |
| | | | | meat (1%) |
| Lecithin | −119% | 8% | 1% | Egg (1%) |

As shown in FIG. 23A, assay performance was unaffected by many of the components tested, including common sweeteners, insoluble fiber, fats, food coloring, salts, and tannins. Variable effects were detected in the presence of acids and alginate (a common thickening agent). High levels of acidity (e.g., pure white vinegar) inhibits the binding of P1-16 to the control panel, to such an extent that a minimum viable signal was never reached. At concentrations above 0.1%, sodium alginate reduced the sensitivity of the assay to peanut by an unknown mechanism. When diluted to lower concentrations, the issues seen for these matrices were resolved, and the number of foods in the problematic range is small.

To confirm the accuracy of our integrated device, we tested a suite of thirty potentially high-risk food matrices that could negatively impact hybridization between the P1-16 aptamer and the test anchors or control anchors. For each food, four replicates were run without peanut (0 ppm) and four replicates were spiked with peanut flour (50 ppm). Additionally, we tested twelve commercially available foods that were known to contain peanut. For each test, the normalized difference (1−test/control) is plotted in FIG. 23B (also see Tables 5 and 6).

Thirty commercially available foods, spiked with 0 ppm or 50 ppm peanut flour, were tested with AF647-P1-16 aptamer. Eight replicates (4 with peanut and 4 without) were run for all foods. Test and control intensities reported as averaged relative fluorescence units. Ratio reported as normalized difference (1−test/control). All foods with 0 ppm peanut flour show a percent decease less than −50%, while all foods with 50 ppm peanut flour show a percent decrease greater than −50%.

TABLE 5
Assay can differentiate between foods
with and without peanut protein.

| | 0 ppm | | | 50 ppm | | |
|---|---|---|---|---|---|---|
| Food | Test | Control | Ratio | Test | Control | Ratio |
| Vanilla Ice Cream | 80 | 41 | −97% | 58 | 45 | −28% |
| Wafer | 113 | 53 | −115% | 36 | 45 | 20% |
| Milky Way | 79 | 40 | −98% | 47 | 43 | −10% |
| Blueberry Gelato | 77 | 45 | −70% | 32 | 42 | 23% |
| Milk Chocolate | 77 | 39 | −95% | 52 | 44 | −18% |
| Mint Chip Ice | 80 | 43 | −88% | 52 | 45 | −14% |
| Cream | | | | | | |
| Nacho Cheese | 82 | 44 | −88% | 45 | 47 | 4% |
| Pasta Sauce | 80 | 43 | −87% | 42 | 39 | −9% |
| Fruity Pebbles | 76 | 41 | −83% | 36 | 43 | 16% |
| Catalina Salad | 81 | 46 | −78% | 34 | 40 | 16% |
| Dressing | | | | | | |
| Mushroom Soup | 86 | 41 | −111% | 39 | 42 | 9% |
| Trix Cereal | 110 | 49 | −128% | 46 | 49 | 4% |
| White Chocolate | 106 | 51 | −109% | 47 | 46 | −2% |
| Applesauce | 98 | 49 | −102% | 46 | 53 | 14% |
| Cheerios | 111 | 52 | −117% | 52 | 49 | −4% |
| Chicken Gravy | 95 | 49 | −96% | 42 | 46 | 9% |
| Hoisin Sauce | 85 | 42 | −101% | 38 | 41 | 8% |
| Cupcake | 71 | 41 | −74% | 34 | 41 | 16% |
| Rice Noodle | 92 | 50 | −84% | 30 | 38 | 28% |
| Vanilla Crispy | 77 | 38 | −103% | 36 | 42 | 13% |
| Square | | | | | | |
| Blue Cheese | 72 | 40 | −80% | 28 | 36 | 23% |
| Dressing | | | | | | |
| Alfredo Sauce | 97 | 48 | −101% | 39 | 44 | 13% |
| Frosting | 79 | 45 | −76% | 34 | 41 | 17% |
| Merengue | 95 | 49 | −95% | 42 | 47 | 12% |
| Fluff | 103 | 53 | −94% | 34 | 44 | 22% |
| Lucky Charms | 96 | 43 | −123% | 50 | 48 | −6% |
| Sauerkraut | 113 | 44 | −158% | 43 | 41 | −5% |
| Blue Gatorade | 76 | 41 | −87% | 38 | 42 | 11% |
| Dots Candy | 93 | 43 | −117% | 35 | 39 | 9% |
| Scrambled Eggs | 109 | 44 | −150% | 39 | 39 | 2% |

Twelve commercially available foods that contain peanut were tested with AF647-P1-16 aptamer. Four replicates were run for all foods. Test and control intensities reported as averaged relative fluorescence units. Ratio reported as normalized difference (1−test/control) (Table 6).

TABLE 6
Foods that contain peanut test positive
for peanut protein in integrated assay

| Food | Test | Control | Ratio |
|---|---|---|---|
| Peanut Butter Ice Cream | 20.3 | 30.6 | 34% |
| Mr. Goodbar | 33.0 | 36.9 | 11% |
| Peanut Dressing | 18.3 | 25.8 | 29% |
| PB Chips Ahoy | 36.8 | 37.5 | 2% |
| PB Wafer | 31.8 | 36.0 | 12% |
| Roasted Peanut Noodle Bowl | 35.6 | 36.7 | 3% |
| PB Captain Crunch | 44.5 | 38.8 | −15% |
| PB Cheerios | 38.2 | 39.2 | 3% |
| PB Mug Cake | 28.0 | 37.1 | 25% |
| PB Yodel | 33.9 | 36.5 | 7% |
| Reese's PB Cup Thins | 19.3 | 25.0 | 23% |
| PB Granola Bar | 28.5 | 35.1 | 19% |

FIGS. 23A-B depict the assay validation. (A) Peanut can be detected in major food components and common food additives. Assay was run using multiple food components and additives, both with and without 50 ppm of peanut flour. Four or five replicates of each peanut flour concentration were tested. (B) Food samples with and without peanut protein can be differentiated by comparing intensity of test spots to control spots. Thirty commercially available foods, spiked with 0 ppm or 50 ppm peanut flour, approximately 5 replicates each, were tested with AF647-P1-16 aptamer. All samples are plotted together versus normalized difference (1−test/control) and labeled only with peanut protein content. A clear distinction is seen between the two populations, represented by the dashed line at −50%. As indicated by the dashed line in the figure, there is clear separation between those samples containing peanut and those without, suggesting that the assay successfully identified peanut contaminated food samples.

### Adaptability

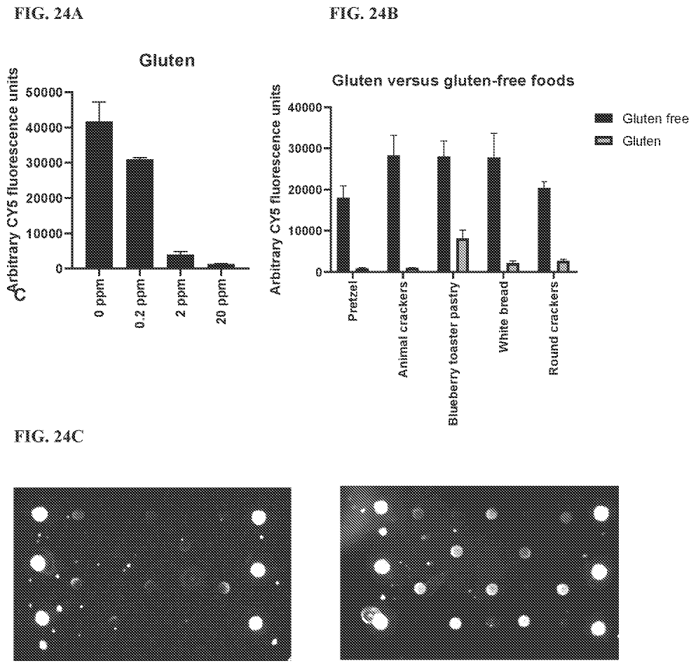

This assay was designed to be extended to any protein target that can be recognized by an aptamer. Consistent with that notion, we have performed preliminary work on an additional allergic target, gluten. Briefly, aptamers that target gluten were chosen by SELEX and screened to hybridize to anchor sequences as described for screening with the P1-16 aptamer. The selected aptamer (GN5) exhibits high sensitivity to gluten, as shown with a dose-dependent curve (FIG. 24A), which demonstrates that the fluorescence intensity is significantly decreased in the presence of 0.2 ppm gluten. We also challenged the GN5 aptamer against commercially available foods. As shown in FIG. 24B, we were able to detect the presence of gluten in commonly consumed foods. FIGS. 24A-C depict future work and that CY5-GN5 aptamer binds to gluten in a concentration dependent manner and in a variety of food matrices. GN5 aptamer was incubated in buffer spiked with increasing concentrations of gluten. FIG. 24B shows commercially available foods (gluten versus gluten-free) were homogenized, filtered, then incubated with GN5. As described for the P1-16 aptamer, the samples were incubated with a chiplet spotted with a 10-oligonucleotide anchor complementary to sequence of GN5. Four replicates of each sample were tested. Error bars represent the standard deviation of the mean. FIG. 24C provides representative images of fluorescent tags alone (top) show low fluorescence, while the combination of fluorescent tag and viral RNA fragment (bottom) shows increased fluorescence. Work is ongoing to design a control anchor, as described for the P1-16 aptamer, to ensure that gluten is detected regardless of the food matrix.

In this work, we designed an aptamer-based assay with the ability to detect targets present in a variety of matrices. By utilizing two different regions of a peanut protein-binding aptamer, we demonstrate that we were able to 1) compete for a peanut protein binding site on the aptamer and 2) hybridize to a different region of the aptamer irrespective of the presence of peanut.

Close investigation of the food testing (Table 5) shows the importance of the internal control in correctly identifying the presence or absence of peanut in an unknown food sample. For example, consider two foods from the list without peanut; white chocolate, with a non-normalized test intensity value of 106 rfus, and cupcake, with a test intensity value of 71 rfus. Because the test intensity for cupcake is 33% lower than that of white chocolate, its dim fluorescence even in the absence of peanut could be taken as a “false positive”, incorrectly identifying the sample as containing peanut. However, the control intensity of cupcake (41 rfus) is also significantly lower than that of white chocolate (51 rfus). By normalizing to the internal control for each sample, the presence or absence of peanut can be correctly identified across a wide variety of foods. The data from the 30 foods analyzed suggests that peanut can be detected in an “unknown” if we establish a threshold value of −50% for the normalized difference. Samples with a normalized difference greater than −50% contain at least 50 ppm peanut flour, while those below that threshold do not. Applying that same threshold to the peanut containing foods, we find that we are able to extract and detect peanut proteins that have been heavily processed. Studies are ongoing to expand the food panel.

Collectively, we detect 50 ppm peanut flour in foods (equal to approximately 12.5 ppm peanut protein), including those in complex matrices, e.g., high protein, high salt, or acidity Work is ongoing to create a robust assay to detect other protein and viral targets and to optimize the gluten-binding aptamer. Overall, these data demonstrate that the novel antigen detection system is extremely adaptable and suitable for rapid transition to additional food allergens as well as in vitro diagnostic applications, such as point-of-care tests.

The platform, which uses a unique combination of aptamer, anchor, and control sequences are able to bind and detect a wide range of targets with high affinity and specificity. Production of these reagents is based on traditional SELEX approaches but boasts additional sensitivity and selectivity and flexibility in design due to the advances described herein. Compared to antibodies, the proposed approach features several practical advantages, including that the reagents are (1) synthesized and chemically modified in a fast, reproducible, and scalable process; (2) small and inexpensive to manufacture with reproducible production characteristics; and (3) stable over a range of temperatures and pH values. These features ensure the device will be stable in extreme environments (a hot car) and useful across complex food matrices. The all-in-one detection platform also represents and end-to-end solution, involving (1) sample collection, (2) validated homogenization and filtration methods, including a universal extraction step that can be applied to food as well as other matrices (sputum, saliva, etc.), and (3) a precision optical sensor and algorithm with built-in controls.

The advantages of the proposed approach are best illustrated through comparison to competing commercial devices available for allergen detection in food. First, many commercially available allergen detection kits currently on the market rely on polymerase chain reaction (PCR), antibody, or mass spectroscopy based technology. These different techniques each have unique limitations. The limitations of PCR assays are especially important to note. A main obstacle to the widespread use of PCR-based allergen or viral detection is the laboratory scale equipment and trained professionals required to perform assays. These limitations not only affect throughput and increase expense, but also introduce considerable inter-operator and inter-laboratory variability. These same limitations apply to antibody- and mass-spec-based tests. Antibody-based detection methods are also limited, particularly by high cross-reactivity, restricted scalability, and instability at high temperatures.

### Selection/Description of Aptamers/SELEX

A random 76-mer library (random region of 30 nucleotides flanked by 23 nucleotide primer regions) was subjected to 10 rounds of positive SELEX with decreasing concentrations of gluten (Sigma-Aldrich), followed by 7 rounds of counter SELEX against mixtures of proteins including common wheat replacements. The pool was isolated and amplified at the end of each round. At the end of 17 rounds, the enriched pool was sent for sequencing. Twelve of the top hits were synthesized and evaluated, with GN5 being selected for best sensitivity and specificity.

### Affinity Measurements

Fluorescence polarization (FP) was used to determine dissociation constants (Kds) for PT-31, P1-10, P1-16, P2-8, and P2-18 interaction with potential targets. The aptamers were synthesized from Integrated DNA Technologies with a Texas Red fluorophore attached to its 5′ end in order to measure changes in fluorescence polarization. Each experiment was performed on a TECAN Spark 10M plate reader (excitation 570 nm/emission 625 nm) set to 5 kinetic cycles. Samples were prepared in 50 uL with FP buffer (50 mM Tris-HCl, 0.1% Tween-20, pH 9) containing 5 nM aptamer and increasing concentration of purified AraH1 (Indoor Biotechnologies) or peanut matrix (Teddie brand unsalted peanut butter or Protein Plus brand roasted natural peanut flour) ranging from 0 to 50 uM and incubated for 10 minutes prior to reading on the spectrofluorometer. Peanut matrices were prepared by homogenizing samples at a stock concentration of 100,000 parts per million in FP buffer and clarifying by centrifugation at 5,000×g for three minutes. Nonlinear regression analyses were used to determine Kds (Prism 8, GraphPad).

### Anchor Screening

CY5-labeled aptamers and 40 short DNA anchors with a ten oligonucleotide sequence complementary to the aptamers were synthesized at Integrated DNA Technologies. Half of the anchor sequences contained a poly-A tail and all anchor sequences contained an amine linker at the 5′ end of the oligonucleotide. Each anchor was spotted on epoxysilane-treated slides at various concentrations (1-40 uM) at Applied Microarrays (Tempe, Arizona). Each slide was pre-blocked with 1% bovine serum albumin in HEPES for two minutes prior to incubation of CY5-aptamer/peanut flour mixture. Aptamer was mixed with peanut flour at different concentrations and allowed to incubate in binding buffer prior to loading onto the well. After a two-minute incubation with mild shaking on an orbital rotator, the slides were washed with binding buffer and scanned for fluorescent CY5 signal. After selection of the 5′-(Amine-6C)-anchor, the linker was extended by six additional carbon atoms and printed at 5 uM concentration on epoxysilane-treated slides for confirmation.

### Assay Details

COP chips were placed in an air-tight chamber with fluidic channels connected to reservoirs from which wash solution or filtered food homogenate were drawn via a pump system. First, 100 uL of wash solution (20 mM Trizma base, 0.2% Brij-L4, 0.2% Capstone FS-31, 0.25 mM MgCl₂) was delivered to the chamber, followed by a short air purge. Then, 100 uL of test sample (containing homogenization buffer (20 mM EPPS, 0.2% Brij-58, 2% PEG 8000, 2% Pluronic F-127, pH 8.4), 15 nM AF647-P1-16, and 30 mM MgCl₂) was delivered to the chamber at a rate of 1000 uL/min. The chamber was cleared, an image captured, and the intensity of the control spots assessed. If less than 30 rfus, another aliquot of 100 uL of test sample was delivered. The process was repeated until the intensity of the control spots is greater than 30 rfus. Then the chamber was washed with 200 uL of wash solution and imaged.

### Guard Band Study

For the matrix interference studies, 15 nM AF647-P1-16 was incubated briefly with the listed additives and components in homogenization buffer (20 mM EPPS, 2% Pluronic F-127, 2% PEG 8000, 0.2% Brij-58, pH 8.4). The percentage represents the amount in a food sample, meaning for a value of 100%, 0.1 g of component was added to 3 mL of assay buffer. Peanut flour was then added to the 50 ppm samples, and the assay was run as described above.

### Specificity Studies

AF647-P1-16 aptamer (20 nM) was incubated with increasing concentrations of purified AraH proteins (Indoor Biotechnologies, AraH1, #NA-AH1-1; AraH2, #NA-AH2-1; AraH3, #NA-AH3-1; AraH6, #NA-AH6-1; AraH8, #RP-AH8-1) in assay buffer and 30 mM MgCl₂. Commercially-available nut flours (pecan, walnut, pistachio, hazelnut, almond, sunflower, and cashew) were homogenized with assay buffer and clarified by centrifugation at 5,000×g for three minutes. To study the specificity of P1-16 aptamer when tree nut is present, 0 or 50 ppm peanut flour was spiked into clarified 50 ppm tree nut flour in assay buffer or 0.1% non-fat milk (dry powder, American Bio). Samples were assayed as described for the guard band study.

### Matrix Testing to Validate Assay

Chips were printed with both 12.5 uM P1-16 anchor and 7 uM of the control anchor. Foods were sampled at 0.1 g and homogenized for 45 s in 3 mL of assay buffer with 15 nM P1-16. For peanut containing samples, 30 uL of a 5000 ppm peanut homogenate was also added. Finally, 30 mM MgCl₂ was added to all samples. The food homogenate was filtered through a 60 um PET mesh. The assay ran as described above. The foods tested were: vanilla ice cream (Edy's), sugar-free vanilla wafer (Voortman Bakery), Milky Way (Mars), Vanilla Blueberry Gelato (Talenti), milk chocolate (Hershey's), mint chocolate chip ice cream (Friendly's), nacho cheese (Tostitos Salsa con Queso Medium), pasta sauce (Stop & Shop), Fruity Pebbles (Post), Catalina Salad Dressing (Kraft), mushroom soup (Campbells condensed, prepared as directed), Trix cereal (General Mills), white chocolate (Ghiradelli), applesauce (Motts Unsweetened Apple), Cheerios (General Mills), chicken gravy (McCormick), hoisin sauce (House of Tsang), Little Bites cupcakes (Entenmann's), rice noodles (A Taste of Thai, cooked until tender), vanilla crispy squares (Made Good), blue cheese dressing (Ken's Steak House Blue Cheese with Gorgonzola Dressing), alfredo sauce (Classico Creamy), frosting (Betty Crocker Frosty White Whipped Topping), pink meringue (Spaans Cookie Company), fluff (Durkee-Mower), Lucky Charms (General Mills), sauerkraut (Stop & Shop), Gatorade (PepsiCo), DOTS (Tootsie fruit flavored drops), and scrambled eggs (generic, cooked until set).

### Gluten Assay

GN5 aptamer was incubated in gluten assay buffer (GAB, 15.4 mM MES buffer, 0.08% Tween-20, 30% ethanol, and 1 mM MgCl₂, pH 5) with increasing concentrations of gluten. Gluten (wheat source, Sigma Life Science) was extracted in GAB and diluted in GAB with 20 nM GB1. For the food testing, commercially available foods were paired with the closest match for the gluten-free counterpart: Round crackers: Ritz brand (wheat) versus Glutino brand gluten-free round crackers (corn starch and rice flour); Kellogg brand (wheat) frosted blueberry toaster pastry versus Glutino brand gluten-free frosted blueberry toaster pastry (rice flour); Snyder's brand pretzel sticks (wheat) versus Snyder's brand gluten free pretzel stick (corn and potato starches); Arnold brand country white bread (wheat) versus Udi's gluten-free white bread (pea, tapioca, and rice starches); Mondeléz International brand animal crackers (wheat) versus Kinnikkinnick kinniKritters animal crackers (pea and potato starches) were tested with GN5. Each food was prepared as described in the matrix testing description, however after filtration, the food filtrate was diluted by an additional 1:10 with GAB. For both assays, 250 uL of GAB is delivered to the chamber, followed by a short air purge. Then, 500 uL of test sample (GAB and 20 nM GN5, with or without gluten) is delivered to the chamber at a rate of 1000 uL/min. Then the chamber was washed with 525 uL of GAB with 10 mM MgCl₂ with the same flow rate. The chips were then air dried and imaged.

### Temperature-Based Stability Experiments

Alexa Fluor 647-labeled P1-16 was placed in a thermocycler and incubated at 95° C., 72° C., or 60° C. (ramped to the target temperature at a rate of 2° C./second) for 10 minutes, and then cooled to 4° C. at a rate of 2° C./second. Each aptamer was then tested in the described assay and compared to a control aptamer sample, which was only exposed to ambient temperature.

### Long-Term Stability Experiments

AF647-P1-16 (10 nM) was formulated in autoclaved homogenization buffer (20 mM EPPS, 0.2% Brij-58, 2% PEG-8000, 2% Pluronic F-127, pH8.4) under aseptic, sterile environmental conditions within a clean room facility. Aliquots of such samples were subjected to accelerated aging at 37° C. At each time point, samples were subjected to intensity over spots measurement assays, and comparisons made with respect to age-matched fresh P1-16.

### Example 4: System Accuracy

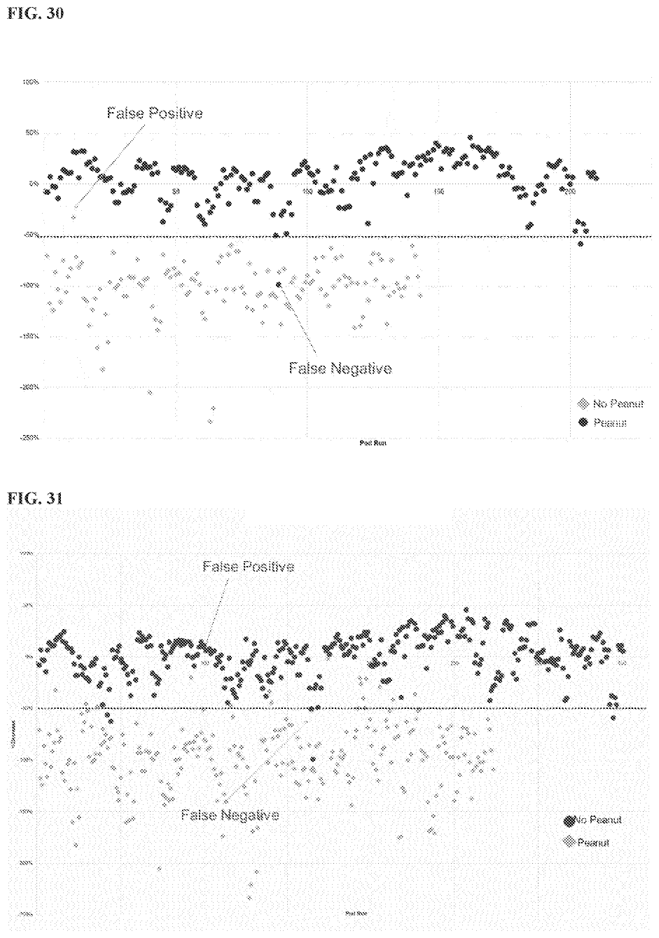

In an assay using a 45-food data set addressing consumer interest and AOAC suggest food categories, the system of the disclosure achieved 99% accuracy. The results shown in Table 7, and FIG. 30 highlight the sensitivity of the system. The assay used 349 analytical cartridges or pods for 45 different food, detecting for peanuts.

TABLE 7
45-Food Accuracy

| | |
|---|---|
| # Foods tests (#pods) | 45 (349) |
| Sensitivity | 99% |
| Specificity | 99% |
| Accuracy | 99% |
| Likelihood ratio of negative test | 136 |
| Likelihood ratio of positive test | 0.015 |
| Positive predictive value | 100% |
| Negative predictive value | 98% |

In an assay using a 70-food data set addressing consumer interest and AOAC suggested chemically and mechanically difficult food categories, the system of the disclosure achieved 96% accuracy. The results shown in Table 8, and FIG. 30 highlight the sensitivity of the system. The assay used 620 analytical cartridges or pods for 70 different food, detecting for peanuts.

TABLE 8
70-Food Accuracy

| | |
|---|---|
| # Foods tests (#pods) | 70 (620) |
| Sensitivity | 99% |
| Specificity | 93% |
| Accuracy | 96% |
| Likelihood ratio of negative test | 14.6 |
| Likelihood ratio of positive test | 0.016 |
| Positive predictive value | 95% |
| Negative predictive value | 98% |

In light of the tested data, the system has a 99% accuracy at a threshold of 12.5 ppm of peanut protein. The system may be identified as high interest by peanut-allergic-consumer food brands to represent the broadest coverage of AOAC food categories.

### Example 5: Testing Alternative Matrices

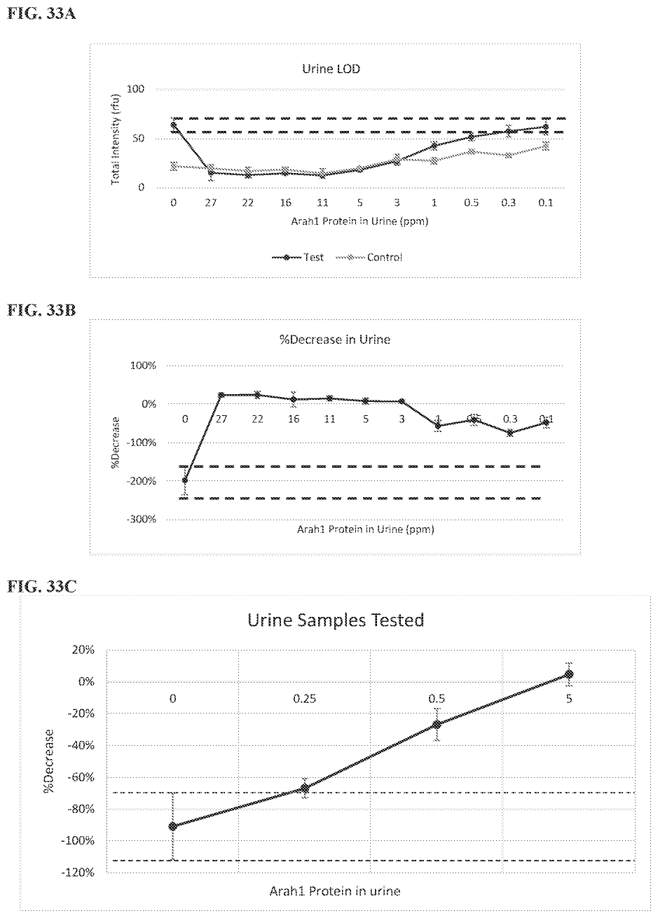

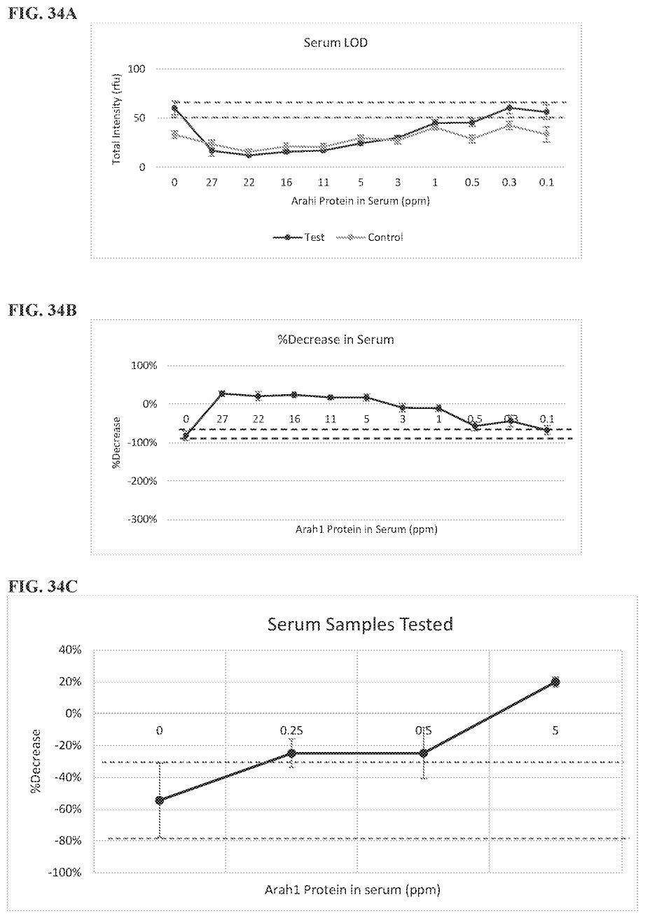

Except food samples, clinical samples, including saliva, urine, serum and stool were tested for protein detection using the present detection system. 1 ml urine sample spiked with different concentrations of AraH1 protein were processed and tested using the present system (FIG. 1). The urine samples were processed in the pod (300) and run through the device (100) to detect the signals (FIGS. 33A-C). 1 ml serum sample spiked with different concentrations of AraH protein were processed and tested using the present system (FIG. 1). The serum samples were processed in the pod (300) and run through the device (100) to detect the signals (FIGS. 34A-C).

As shown in FIGS. 33C and 34C, Arah1 protein LOD on the present system indicates 0.1 ppm AraH1 protein in urine and 0.3 ppm AraH1 in serum (average standard deviation: 10%). The detecting results indicate that the present system can detect Arah1 protein in urine and serum at a level of 0.08-0.17 ppm in the pod (300), which is comparable to the level of AraH1l protein in foods (at a level of 1.25-1.5 ppm in the pod).

The detection threshold of Arah1 protein in serum and urine at concentrations as low as 0.1 ppm in 3 minutes is clinically relevant. The clinical relevancy achieved with the present system is comparable with other diagnostic assays for other clinical targets, e.g., targets shown in Table 9.

TABLE 9
Clinical targets detection threshold

| | | | Detection | Current | Instrument/ |
|---|---|---|---|---|---|
| Target | Indication | Matrix tested | Range (ppm) | Testing time | Kit |
| Hemoglobin | Screen for | Serum | 50,000-250,000 | 2 | i-STAT Abbott |
| | Anemia | | ppm | min | |
| Cholesterol | Screen for | Serum | >2000 | 5 | Cholestech |
| | heart disease | | ppm | min | LDX ™ Analyzer |
| Transferrin | Screen for | Serum | 1920-3640 | 100 immune | Abbott Architect |
| | Iron | | ppm | tests/hour | Core laboratory |
| | deficiency | | | | |
| A1C | Screen for | Serum | 1520 | 100 immune | Abbott Architect |
| | Diabetes | | ppm | tests/hour | Core laboratory - |
| | | | | | metabolic panel |
| Glucose | Screen for | Serum | 200-7000 | 2 | i-STAT Abbott |
| | Diabetes | | ppm | min | |
| Prealbumin | Screen for | Serum | 140-350 | 100 immune | Abbott Architect |
| | protein | | ppm | tests/hour | Core laboratory - |
| | metabolism | | | | protein panel |
| Creatinine | Renal disease | Serum | 20-200 | 2 | i-STAT Abbott |
| | and | | ppm | min | |
| | monitoring of | | | | |
| | renal dialysis | | | | |
| DHEA- | Screen for | Serum | 1.38-4.75 | 100 immune | Abbott Architect |
| Sulfate | fertility | | ppm | tests/hour | Core laboratory - |
| | | | | | fertility panel |
| CK-MB - | myocardial | Plasma/whole | 0-0.15 | 5 | i-STAT Abbott |
| creatine | infarction | blood | ppm | min | |
| kinase MB | | | | | |
| β-hCG | Early | Plasma/whole | 0.005-2 | 10 | i-STAT Abbott |
| | detection of | blood | ppm | min | |
| | pregnancy | | | | |
| Cortisol | Screen for | Serum | 0.02-0.12 | 100 immune | Abbott Architect |
| | Adrenal | | ppm | tests/hour | Core laboratory - |
| | gland | | | | metabolic panel |
| | disorders | | | | |
| cTnl- Cardiac | Myocardial | Plasma/whole | 0-0.05 | 10 | i-STAT Abbott |
| Troponin | infarction and | blood | ppm | min | |
| | risk | | | | |
| | stratification | | | | |
| | of patients | | | | |
| | with acute | | | | |
| | coronary | | | | |
| | syndromes | | | | |

1. An assembly for detecting a molecule of interest in a sample comprising:
a sample processing cartridge having a homogenization chamber configured to accept the sample for processing to a state permitting the molecule of interest to engage in an interaction with a detection agent; the cartridge comprising:
a lid,
a movable cap, the movable cap having a lancing element, and
a homogenization accelerator, which is secured in a pocket bounded by the cap and a seal on the lid;
a detector unit configured to accept the sample processing cartridge in a configuration which permits a detection mechanism housed by the detector unit to detect the interaction of the molecule of interest with the detection agent, wherein the interaction triggers a visual indication on the detector unit that the molecule of interest is detected; and
wherein the visual indication is by processing images capturing the interaction of the molecule of interest with the detection agent.

2. The assembly of claim 1, wherein the movable cap is rotatably secured to the lid.

3. The assembly of claim 1, wherein the lid further comprises at least one aperture opening into the homogenization chamber.

4. The assembly of claim 3, wherein the pocket is co-located with the at least one aperture.

5. The assembly of claim 4, wherein movement of the movable cap causes the lancing element to lance the seal allowing the homogenization accelerator to enter the homogenization chamber.

6. The assembly of claim 3, wherein the at least one aperture further includes a second aperture opening into the homogenization chamber.

7. The assembly of claim 6, wherein the cap further includes a port which, in a first position of the cap, co-localizes with the second aperture; the second aperture containing a breakable seal facing the homogenization chamber.

8. The assembly of claim 7, wherein in a second position of the cap the second aperture is covered by the cap and sealed by a movable cover.

9. The assembly of claim 1 wherein the molecule of interest is an allergen, or a clinical target.

10. The assembly of claim 9 wherein the detection agent is an antibody or variant thereof, a nucleic acid molecule or variant thereof, or a small molecule.

11. The assembly of claim 10, wherein the detection agent is a nucleic acid molecule or variant thereof.

12. The assembly of claim 11, wherein the nucleic acid molecule is an aptamer that comprises a nucleic acid sequence that binds to the molecule of interest, or a signaling polynucleotide (SPN) derived from said aptamer.

13. The assembly of any one of claims 1 to 12 wherein the sample processing cartridge comprises:
a homogenizer configured to produce a homogenized sample, thereby releasing the molecule of interest from a matrix of the sample into an extraction buffer in the presence of the detection agent;
a plurality of separate chambers including the homogenization chamber, a filtrate chamber, and a detection chamber;
a first conduit to transfer the homogenized sample and detection agent through a filter system to provide a filtrate containing the molecule of interest and the detection agent; and
a second conduit to transfer the filtrate to a detection chamber with a window;
wherein the detection mechanism of the detector unit analyzes the detection chamber through the window to identify the interaction of the molecule of interest with the detection agent in the detection chamber.

14. The assembly of claim 13 wherein the homogenizer comprises a rotor and wherein the rotor is powered by a motor located in the detector unit, wherein the motor is functionally coupled to the homogenizer when the sample processing cartridge is accepted by the detector unit, and wherein the homogenization accelerator is configured to engage with the rotor to assist in homogenization.

15. The assembly of claim 13 or 14 wherein the sample processing cartridge further comprises a chamber holding wash buffer for washing the detection chamber and a waste chamber for accepting outflow contents of the detection chamber after wash.

16. The assembly of claim 15 wherein the sample processing cartridge further comprises a rotary valve system for controlling transfer of the homogenized sample to the filter system, for transfer of the filtrate to the detection chamber, for transfer of the wash buffer to the detection chamber and for transfer of contents of the detection chamber to the waste chamber.

17. The assembly of claim 16 wherein the rotary valve system is further configured to provide a closed position to prevent fluid movement in the sample processing cartridge.

18. The assembly of any one of claims 13 to 17 wherein the detection chamber includes a transparent substrate with a detection probe molecule immobilized thereon, the detection probe configured to engage in a probe interaction with the detection agent, wherein the interaction of the molecule of interest with the detection agent prevents the detection agent from engaging in the probe interaction with the detection probe.

19. The assembly of claim 18, wherein the assembly further comprises an assembly lid capable of measuring the weight, mass, or volume of a sample.

20. The assembly of claim 19, wherein the assembly lid further comprises:
a frame,
a base attached to the frame, and
a cover connected to the frame; wherein the cover includes a measurement device adjacent thereto and above the base, whereby the measurement device is capable of detecting and measuring the weight, mass, or volume of the sample when the sample is placed on the cover.

21. The assembly of claim 20, wherein the measurement device is a strain gauge or a load gauge.

22. The assembly of claim 21 wherein the transparent substrate further comprises a fluidic panel in connection with the probes for transfer of the filtrate containing the molecule of interest and the detection agent to contact with the detection probe and control probe.

23. The assembly of claim 22, further comprising a sampler, the sampler comprising a hollow tube with a cutting edge for cutting a source to generate and retain the sample within the hollow tube and a plunger for pushing the sample out of the hollow tube and into a port in the sample processing cartridge, the sampler capable of breaking the seal on the second aperture.

24. An analytic cartridge for detecting a molecule of interest in a sample comprising:
a first compartment with a homogenizer for receiving a sample and processing the sample, the homogenizer configured to produce a homogenized sample, thereby releasing the molecule of interest from a matrix of the sample into an extraction buffer in the presence of the detection agent and permitting the molecule of the interest in the sample to engage in the interaction with the detection agent;
a lid covering the cartridge, wherein the lid comprises:
at least one aperture opening into the first compartment,
a cap rotatably connected to the lid, wherein the cap is capable of rotating from a first position to a second position,
a seal on the at least one aperture creating a pocket between the seal and the cap,
a homogenization accelerator positioned in the pocket when the cap is in a first position, and wherein when the cap is rotated to the second position the homogenization accelerator is released into the first compartment;
a conduit to transfer the homogenized sample and detection agent through a filter system to provide a filtrate containing the molecule of interest and the detection agent;
a second compartment for contacting the filtrate containing the molecule of interest and the detection agent with detection probes; the second compartment comprising a transparent substrate that comprises fluidic channels and a detection chip area with a detection probe immobilized thereon, the detection probe configured to engage in a probe interaction with the detection agent, wherein the interaction of the molecule of interest with the detection agent prevents the detection agent from engaging in the probe interaction with the detection probe;
a rotary valve system configured to regulate the transfer of the homogenized sample and detection agent through the filter system, of the filtrate to the second compartment, and of wash buffer to the second compartment and outflow contents from the second compartment to a waste chamber;
a compartment for holding wash buffer for washing the detection area; and
a waste chamber for accepting outflow contents of the detection chamber.

25. The analytic cartridge of claim 24, wherein the at least one aperture further includes a second aperture opening into the first compartment.

26. The analytic cartridge of claim 25, wherein the cap further includes a port which, when the cap is in the first position, co-localizes with the second aperture; the second aperture containing a breakable seal facing the first compartment.

27. The analytic cartridge of claim 26, wherein when the cap is in the second position, the second aperture is covered by the cap and sealed by a movable cover.

28. The analytic cartridge of claim 27 wherein the second compartment comprises a window through which the detection mechanism of a detector unit analyzes the detection reaction through the window to identify the interaction of the molecule of interest with the detection agent in the second compartment.

29. The analytic cartridge of claim 28 wherein the detection area of the transparent substrate further comprises an optically detectable control probe molecule immobilized thereon, for normalization of signal output measured by the detection mechanism.

30. The analytic cartridge of claim 29 wherein the substrate further comprises two different optically detectable control probe molecules immobilized thereon, for normalization of signal output measured by the detection mechanism.

31. The analytic cartridge of any one of claim 24 wherein the detection agent is a nucleic acid molecule comprising a nucleic acid sequence that binds to the molecule of interest.

32. The analytic cartridge of claim 31 wherein the nucleic acid-based detection agent is a signaling polynucleotide (SPN) derived from an aptamer that comprises a nucleic acid sequence that binds to the molecule of interest.

33. The analytic cartridge of claim 32 wherein the detection agent includes an optically detectable fluorescent moiety which is activated when the probe interaction is engaged.

34. The analytic cartridge of claim 23 wherein the cartridge further comprises a plurality of fluid flow paths for transfer of the homogenized sample to the filter system, for transfer of the filtrate to the transparent substrate, for transfer of the wash buffer to the detection compartment and for transfer of contents of the detection compartment to the waste chamber.

35. The analytic cartridge of claim 33 in combination with a detector device, the detection device comprising:
an external housing configured for providing support for the components of the detection device; the components integrated for operating a detection test comprising:
an assembly lid capable of measuring the weight, mass, or volume of a sample,
a motor for driving and controlling the sample homogenization,
a motor for controlling a valve system,
a pump for driving and controlling fluidic flow,
an optical system for detecting fluorescence signals,
means for converting and digitizing the fluorescence signals,
a display window for receiving the detected signals and indicating the presence and/or absence of the allergen in the test sample, and
a power supply.

36. A test cup assembly for processing a sample to a state permitting detection of a molecule of interest in the sample comprising:
a top cover for sealing the test cup and providing an identification label, the top cover further comprising: a movable cap, the movable cap having a lancing element, and a homogenization accelerator, which is secured in a pocket bounded by the cap and a sealed aperture on the top cover;
a body part for receiving and processing the sample to a state permitting the molecule of interest in the sample to engage in an interaction with a detection agent, the body part comprising:
a first compartment with a homogenizer for homogenizing the sample to extract the molecule of interest using an extraction buffer, thereby releasing the molecule of interest from a matrix of the sample into the extraction buffer and engaging in the interaction with a detection agent present in the extraction buffer;
a conduit for transferring the homogenized sample containing the molecule of the interest and detection agent through a filter system to provide a filtrate containing the molecule of interest and the detection agent;
a chamber for holding wash buffer;
a waste chamber for receiving and storing the outcome contents after washing the molecule of interest and the detection agent; and
a rotary valve system for controlling the fluid movement inside the test cup assembly;
a transparent substrate comprising a plurality of fluidic channels and a detection area with a detection probe immobilized thereon, the detection probe configured to engage in a probe interaction with the detection agent, wherein the interaction of the molecule of interest with the detection agent prevents the detection agent from engaging in the probe interaction with the detection probe; and
a bottom cover for sealing the test cup and providing an interface to connect the test cup to a detector unit for operating the detection; the bottom cover comprising a transparent window that is aligned with the detection area of the transparent substrate upon assembly of the test cup.

37. The test cup assembly of claim 36 wherein the cup top cover comprises a port for receiving the sample and at least one breather filter that allows air in.

38. The test cup assembly of claim 37, wherein the cap includes a food aperture that, when the cap is in a first position, aligns with the port of the top cover.

39. A system for detecting the presence or absence of a molecule of interest in a sample, comprising:
a sampler for collecting a sample suspected of containing the molecule of interest;
a disposable analytical cartridge configured for processing the sample, thereby permitting the molecule of the interest in the sample to engage in the interaction with a detection agent; and
a detection device configured for measuring the sample, operating the detection test, and measuring and visualizing a signal from the binding interaction between the detection agent and the molecule of the interest presented in the sample.

40. The system of claim 39 wherein the disposable analytical cartridge comprises:
a lid with a housing and a movable cap, the movable cap having a lancing element, and a homogenization accelerator, which is secured in a pocket bounded by the cap and a seal on the lid;
a sample processing chamber with a homogenizer configured to homogenize the sample with an extraction buffer in the presence of the detection agent, thereby permitting the allergen of the interest in the sample to engage in the interaction with the detection agent;
a filter system configured to provide a filtrate containing the allergen of interest and the detection agent;
a separate transparent substrate comprising a plurality of fluidic channels and a detection area with a detection probe molecule immobilized thereon; the detection probe configured to engage in a probe interaction with the detection agent, wherein the interaction of the molecule of interest with the detection agent prevents the detection agent from engaging in the probe interaction with the detection probe;
a detection chamber with an optical window;
a chamber holding wash buffer for washing the substrate and the detection chamber;
a waste chamber for accepting and storing outflow contents of the detection chamber after wash;
a rotary valve system and conduits configured to transfer the homogenized sample and detection agent through the filter system, to transfer the filtrate to the detection chamber, and to transfer the wash buffer to the detection chamber and outflow contents from the detection chamber to the waste chamber; and
an air flow system configured to regulate air pressure and flow rate in the cartridge.

41. The system of claim 1, wherein the movable cap is rotatably secured to the lid, the lid comprising an aperture opening into the homogenization chamber, the pocket being adjacent with the aperture; wherein movement of the movable cap from a first position to a second position causes the lancing element to lance the seal allowing the homogenization accelerator to enter the homogenization chamber.

42. The system of claim 39 wherein the analytic cartridge further comprises MgCl₂ lyophilized beads.

43. The system of claim 39 wherein the detection device comprises:
a frame attachable to the housing;
a base attached to the frame; and
a cover connected to the frame; and wherein the cover includes a measurement device adjacent thereto and above the base, whereby the measurement device is capable of detecting and measuring the weight, mass, or volume of the sample when the sample is placed on the cover.

44. The system of claim 41 wherein the detection device comprises:
a frame attachable to the housing;
a base attached to the frame; and
a cover connected to the frame; wherein the cover includes a measurement device adjacent thereto and above the base, whereby the measurement device is capable of detecting and measuring the weight, mass, or volume of the sample when the sample is placed on the cover.

45. The system of claim 39 where in the measurement device is a strain gauge.

46. A method for detecting the presence or absence of a molecule of interest in a sample comprising:
collecting a sample, measuring the weight of the sample, homogenizing the sample with an accelerator, and processing the sample in an extraction buffer in the presence of a detection agent, thereby permitting the interaction of the molecule of interest with the detection agent;
filtrating the processed sample containing the molecule of interest and the detection agent;
contacting the filtrate with a substrate with a detection probe immobilized thereon; the detection probe configured to engage in a probe interaction with the detection agent, wherein the interaction of the molecule of interest with the detection agent prevents the detection agent from engaging in the probe interaction with the detection probe;
washing off the unbound compounds from the substrate with a wash buffer;
measuring fluorescence signals from the substrate; and
detecting the presence or absence of the molecule of interest in the sample.

47. The method of claim 46, wherein the molecule of interest is an allergen or a clinical target.

48. The method of claim 47, wherein the sample is a food sample or a clinical sample.

49. The method of claim 48, wherein the clinical sample is urine, serum, plasma, saliva or stool.
